SEQUENCE LISTING (2 sequences)

SEQ ID NO: 1 - DNA, 20 bp, Artificial Sequence
  Description of Artificial Sequence Synthetic primer
gtcccgccttcgacgcacaa

SEQ ID NO: 2 - DNA, 20 bp, Artificial Sequence
  Description of Artificial Sequence Synthetic primer
ggggcggtaggaaagcgtgc

(19) United States
(12) Patent Application Publication
(10) Pub. No.: US 2013/0287688 A1
(43) Pub. Date: Oct. 31, 2013

(54) NOVEL COMPOSITIONS AND USES OF ANTI-HYPERTENSION AGENTS FOR CANCER THERAPY

(71) Applicants: The General Hospital Corporation, (US); Xtuit Pharmaceuticals, Inc., (US)

(72) Inventors: Rakesh Kumar Jain, Wellesley, MA (US); Yves Boucher, Belmont, MA (US); Vikash Pal Singh Chauhan, Cambridge, MA (US); Benjamin Diop-Frimpong, Boston, MA (US); Stephen Krane, Cambridge, MA (US); Alan L. Crane, Newton, MA (US); Robert Langer, Newton, MA (US)

(73) Assignees: XTUIT PHARMACEUTICALS, INC., Waltham, MA (US); THE GENERAL HOSPITAL CORPORATION, Boston, MA (US)

(21) Appl. No.: 13/834,094

(22) Filed: Mar. 15, 2013

Related U.S. Application Data
(63) Continuation-in-part of application No. PCT/US2011/061510, filed on Nov. 18, 2011.
(60) Provisional application No. 61/415,192, filed on Nov. 18, 2010.
(60) Provisional application No. 61/438,240, filed on Jan. 31, 2011.
(60) Provisional application No. 61/643,487, filed on May 7, 2012.

(51) Int. Cl. A61K 31/4178; A61K 31/704; A61K 45/06; A61K 31/4184; A61K 31/41; A61K 31/745; A61K 35/76

(52) U.S. Cl. CPC A61K 31/4178; A61K 31/745; A61K 31/704; A61K 35/763; A61K 31/4184; A61K 31/41; A61K 45/06
USPC 424/9.1; 514/381; 424/78.18; 514/34; 424/93.6; 424/489; 514/616; 514/400; 514/212.07; 514/423; 514/91; 514/311; 514/412; 514/21.9; 514/21.6; 514/21.8; 424/158.1; 514/267; 514/47; 514/1.1; 514/274; 548/252; 548/253; 435/7.92; 428/402

(57) ABSTRACT

Methods and compositions for improving the delivery and/or efficacy of a therapy (e.g., a cancer therapy) are disclosed. In one embodiment, methods and compositions for treating or preventing a cancer (e.g., a solid tumor such as a desmoplastic tumor) by administering to a subject an anti-hypertensive agent, as a single agent or in combination with a microenvironment modulator and/or a therapy, e.g., a cancer therapy (for example, a therapeutic agent or therapy, including immunotherapy (e.g., antibodies, vaccine, cell-based), nanotherapeutics, radiation therapy, photodynamic therapy, low molecular weight chemotherapeutics, molecularly targeted therapeutics and/or oxygen radical) are disclosed.

### CROSS REFERENCE TO RELATED APPLICATIONS

This application is a continuation-in-part application of International Application No. PCT/US2011/061510, filed Nov. 18, 2011, published as International Publication No. WO 2012/068531 on May 24, 2012, and which claims the benefit of priority to U.S. Ser. No. 61/438,240, filed on Jan. 31, 2011, and U.S. Ser. No. 61/415,192, filed on Nov. 18, 2010. This application also claims the benefit of priority to U.S. Provisional Application Ser. No. 61/643,487, filed May 7, 2012. The contents of the aforementioned applications are hereby incorporated by reference in their entirety.

### GOVERNMENT SUPPORT

This invention was made with federal funding under Grant No. P01-CA-80124 awarded by the National Institutes of Health. The U.S. government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 14, 2013, is named 030258069093-CIP and is 829 bytes in size.

### BACKGROUND

Advances in biomedical research have led to the introduction of several novel systemically administered molecular and nanotherapeutic agents in both preclinical and clinical settings (Jones, D. (2007) Nat Rev Drug Discov 6, 174-175; Moghimi, S. M. et al. (2005) Faseb J 19, 311-330). While these new agents act on unique targets that afford greater specificity to tumor cells or improved pharmacodynamic properties, their effectiveness suffers from limitations in their delivery owing to the properties of the tumor microenvironment (Jain, R. K. (1998) Nat Med 4, 655-657; Sanhai, W. R. et al. (2008) Nat Nanotechnol 3, 242-244; Chauhan, V et al. (2011) Annu Rev Chem Biomol Eng. 2(1):281-98). For example, pegylated liposomal doxorubicin (DOXIL®), approved by the FDA, and oncolytic viruses, currently in multiple clinical trials, represent two nanotherapeutics whose size (˜100 nm) hinders their intratumoral distribution and therapeutic effectiveness (Nemunaitis J, et al. (2001) J Clin Oncol 19:289-298).

At least two processes governing drug delivery following systemic administration, namely, vascular transport throughout tissues and transvascular transport into tissues, are hindered by physiological barriers in tumors for all classes of therapeutics (Jain, R. K. & Stylianopoulos (2010) Nat Rev Clin Oncol. 139; Chauhan, V. P. et al. (2009) Biophysical Journal 97, 330-336). These barriers impact therapy, particularly, for patients with desmoplastic, fibrotic tumors, such as pancreatic (Olive, K. P. et al. (2009) Science 324, 1457-1461), colorectal (Halvorsen, T. B. & Seim, E. (1989) J Clin Pathol 42, 162-166), lung and breast cancer (Ronnov-Jessen, L. et al. (1996) Physiol Rev 76, 69-125). Fibrotic tumors typically have a dense collagen network, which causes small interfibrillar spacing in the interstitium to retard the movement of particles larger than 10 nanometers (Netti P A, et al. (2000) Cancer Res 60:2497-2503; Pluen A, et al. (2001) Proc Natl Acad Sci USA 98:4628-4633; Ramanujan S, et al. (2002) Biophys J 83:1650-1660; and Brown E, et al. (2003) Nat Med 9:796-800) These barriers limit the amount of drug that reaches the target cancer cells and leads to poor drug effectiveness.

Currently, there are limited approaches to overcome these delivery barriers for nanotherapeutics and for low molecular weight drugs. Thus, the need exists for identifying new cancer therapies, in particular new agents that enhance the delivery and distribution of cancer therapies, including nanotherapeutics (e.g., lipid- or polymeric nanoparticles and viruses), protein and nucleic acid drugs, small molecule chemotherapeutic agents and immune cells.

### SUMMARY OF THE INVENTION

The invention is based, in part, on the discovery that losartan, an angiotensin II receptor antagonist drug approved for the treatment of high blood pressure (hypertension), improves the delivery and efficacy of cancer therapeutics. The inventors have discovered, inter alia, that losartan normalizes the collagen, interstitial matrix of solid tumors and facilitates the distribution and/or penetration of chemotherapeutics, including large molecular weight chemotherapeutics, e.g., nanotherapeutics. For example, losartan reduced collagen I levels in (e.g., reduced collagen production by) carcinoma associated fibroblasts (CAFs) isolated from breast cancer biopsies, and caused a dose-dependent reduction in stromal collagen in desmoplastic models of human breast, pancreatic and skin tumors in mice. Losartan also improved the distribution, therapeutic efficacy and/or penetration of nanopartices (e.g., oncolytic herpes simplex viruses (HSV) and pegylated liposomal doxorubicin (DOXIL®)). The inventors have also discovered that losartan facilitates decompression of blood vessels and vascular normalization, and improves tumor perfusion and delivery of low molecular weight chemotherapeutics and oxygen, thus enhancing the therapeutic effect of cancer therapies, including but not limited to radiation, photodynamic therapy, chemotherapeutics and immunotherapies. Examples disclosed herein further demonstrate a reduction in collagen levels and tumor solid stress using angiotensin inhibitors other than losartan, including, for example, angiotensin receptor blockers (ARBs), such as candesartan and valsartan, as well as angiotensin converting enzyme inhibitors (ACE-I), such as lisinopril.

Thus, methods and compositions for improving the delivery and/or efficacy of therapeutics (e.g., cancer therapeutics) are disclosed. Methods and compositions for treating or preventing a cancer (e.g., a solid tumor such as a desmoplastic tumor) by administering to a subject an anti-hypertensive and/or collagen modifying agent, as a single agent or in combination with a microenvironment modulator, and/or a therapeutic agent (for example, a cancer therapeutic agent ranging in size from an immune cell or a large nanotherapeutic to a low molecular weight chemotherapeutics and/or oxygen radicals) are disclosed.

Accordingly, in one aspect, the invention features a method of treating or preventing a disorder, e.g., a hyperproliferative disorder (e.g., a cancer) in a subject, or of improving the delivery and/or efficacy of a therapy (e.g., a cancer therapy) to a subject. The method includes:

administering an anti-hypertensive and/or a collagen modifying agent (referred to herein as “AHCM” or “AHCM agent”) to the subject;

optionally, administering a microenvironment modulator; and

optionally, administering the therapy (e.g., the cancer therapy),

under conditions, e.g., of dosage of AHCM and anti-cancer agent, sufficient to treat or prevent the disorder (e.g., the cancer or tumor), in the subject, or to improve the delivery and/or efficacy of the therapy (e.g., the cancer therapy) provided to the subject.

In one embodiment, the method includes one or more of the following:

a) selecting or identifying the subject as being in need of receiving the AHCM or microenvironment modulator (or both) on the basis of the need for improved delivery and/or efficacy of the therapy (e.g., the cancer therapy);

b) administering the AHCM, the microenvironment modulator, or the therapy (e.g., the cancer therapy), or any combination thereof, as an entity having a hydrodynamic diameter of greater than about 1, 5, 10, 15, 20, 25, 30, 35, 45, 50, 75, 100, 150, 200 nm, but less than 300 nm, e.g., as a nanoparticle;

c) the subject has a history of treatment (or lack of treatment) for hypertension, as described herein, e.g., the subject has not been administered a dose of an AHCM, e.g., an AHCM named herein, or any AHCM (e.g., either of a dose sufficient to substantially lower the subject's blood pressure or a sub-anti-hypertensive dose), within 5, 10, 30, 60 or 100 days of the diagnosis of cancer or the initiation of the AHCM dosing. In one embodiment, the subject is not hypertensive, or has been hypertensive, prior to administration of the AHCM;

d) treating the subject with a dosing regimen described herein, e.g., administration of the AHCM and/or the microenvironment modulator is initiated prior to the initiation of administration of the cancer therapy, e.g., it is initiated at least one, two, three, or five days, or one, two, three, four, five or more weeks prior to cancer therapy (e.g., the AHCM and/or the microenvironment modulator is administered at a minimum of two weeks prior to cancer therapy);

e) providing the AHCM, and/or the microenvironment modulator, and the cancer therapy according to a dosing regimen described herein, e.g., providing a first course of treatment with an ARCM at a sub-anti-hypertensive dose followed by a second, higher dose, course of treatment with an AHCM, e.g., at a dose that is at or above a standard anti-hypertensive dose (e.g., wherein the second course is administered in a time course that will counteract a hypertensive affect of an anti-cancer therapy);

f) administering the AHCM and/or the microenvironment modulator substantially continuously over a period of at least 1, 5, 10, or 24 hours; at least 2, 5, 10, or 14 days; at least 2, 3, 4, 5 or 6 weeks; at least 2, 3, 4, 5 or 6 months; or at least 1, 2, 3, 4 or 5 years, or longer;

g) administering the AHCM and/or the microenvironment modulator sequentially and/or concurrently with the therapy, e.g., the cancer therapy. The AHCM, the microenvironment modulator and the therapy can be administered (at the same or different dosages) in any order and/or overlap with the therapy. In one embodiment, the AHCM and/or the microenvironment modulator is administered before the therapy (e.g., as described in step d)). In other embodiments, the ARCM and/or the microenvironment modulator is administered sequentially and/or concurrently with the therapy (e.g., the AHCM and/or the microenvironment modulator is administered prior to the therapy (e.g., as described in step d) and concurrently with the therapy). In yet other embodiments, the therapy is administered first, and the AHCM and/or the microenvironment modulator is administered after initiation of the therapy, or is administered after cessation of the therapy. In other embodiments, the administration of the AHCM and/or the microenvironment modulator starts after cessation of the therapy. In other embodiments, the administration of the AHCM and/or the microenvironment modulator continues after cessation of the therapy. In embodiments where administration of the AHCM and/or the microenvironment modulatorand the therapy is concurrent, the administration of the AHCM, the microenvironment modulator and the therapy can be continued as clinically appropriate, for example, (i) as a combination therapy, (ii) with a period of therapy with either the AHCM or the therapy, or (iii) as a combination of (i) and (ii) in any order.

In one embodiment, the ARCM and/or the microenvironment modulator alters (e.g., enhances), (e.g., is administered in an amount sufficient to alter (e.g., enhance)), the distribution or efficacy of the therapy, e.g., the cancer therapy. In some embodiments, the ARCM does not inhibit or prevent (e.g., is administered in an amount insufficient to inhibit or prevent) tumor growth by itself, but sufficient to alter (e.g., enhance) the distribution or efficacy of the therapy, e.g., the cancer therapy.

In an embodiment, the AHCM results in (e.g., is administered at a dose that causes), one or more of: decreases the level or production of an extracellular matrix component, such as a fiber (e.g., collagen, procollagen), and/or a polysaccharide (e.g., a glycosaminoglycan such as hyaluronan or hyaluronic acid); decreases the level or production of collagen or procollagen; decreases the level or production of hyaluronic acid; decreases tumor fibrosis; increases interstitial tumor transport; improves tumor perfusion; increases tumor oxygenation; decreases tumor hypoxia; decreases tumor acidosis; enables immune cell infiltration; decreases immunosuppression; increases antitumor immunity; decreases the production of cancer stem cells (also referred to herein as tumor-initiating cells); or enhances the efficacy (e.g., penetration or diffusion), of the therapy, e.g., the cancer therapy (e.g., radiation, photodynamic therapy, chemotherapeutics and immunotherapies) in a tumor or tumor vasculature, in the subject.

In an embodiment, the AHCM and/or microenvironment modulator is administered in a dosage sufficient to improve the delivery or effectiveness of the therapy.

In an embodiment the method results in, or comprises (e.g., the AHCM and/or microenvironment modulator is administered in a dosage sufficient to result in) improvement of a disorder-related parameter in said subject, as compared to a subject treated with said therapy but without administration of the AHCM and/or microenvironment modulator. “Disorder-related parameter,” as used herein, refers to a parameter that varies with the alleviation of the disorder or a symptom of the disorder.

In an embodiment, an AHCM (and, in embodiments not a microenvironment modulator) is administered and the improvement is as compared to a subject treated with said therapy but without administration of the AHCM.

In an embodiment, a microenvironment modulator (and, in embodiments not an AHCM) is administered and the improvement is as compared to a subject treated with said therapy but without administration of the microenvironment modulator.

In an embodiment, an AHCM and a microenvironment modulator are administered and the improvement is as compared to a subject treated with said therapy but without administration of the AHCM and the microenvironment modulator.

In an embodiment, the parameter comprises relief of a symptom of said disorder.

In an embodiment, the parameter comprises outcome of a patient scored evaluation of symptoms or quality of life, e.g., a quality of life questionaire, e.g., outcome on an evaluation of number of meals consumed on the day prior to the evaluation, pain, weight loss or gain.

In an embodiment, the parameter comprises one or more or all of:

a) objective response rate (ORR);

b) progression free survival (PFS);

c) overall survival (OS), or

d) reduction in toxicity (whether or not accompanied by an increase on OS. ORR evaluations will differ between disorder but such evaluations are within the skill of the art. For an example, see Willett et al. (2009) Journal of Clinical Oncology 27: 3020-6, which discusses the use of pathological response estimated from evaluation of tissue after surgical resection. Evaluation of PFS is within the skill of the art. For an example, for brain tumors, APF6 (alive and progression-free at 6 months) has been used, see Batchelor et al. (2010) J. Clinical Oncology 28: 2817-23. For radiation therapy, criteria such as “Disease-free survival” and “Freedom from metastasis” have been used, see, e.g., Willett et al, 2010, The Oncologist, 15:845-851. Some evaluations of ORR and PFS rely on imaging methods, e.g., PET, PET-MRI, or PET-CT. Evaluation of toxicity will vary by disorder and treatment modality. One example can be seen in Willett et al. (2010) The Oncologist 15:845-851.

In an embodiment, the parameter comprises one or more or all of: a) drug concentration, e.g., at a disorder or disease site, e.g., in a solid tumor; b) tumor response; c) blood perfusion, e.g., at a disorder or disease site, e.g., in a solid tumor; d) oxygenation, e.g., at a disorder or disease site, e.g., in a solid tumor; e) interstitial fluid pressure at a disorder or disease site, e.g., in a solid tumor; or f) extracellular matrix content or composition, e.g., level of collagen, hyaluronic acid.

In an embodiment, the parameter is evaluated by a non-invasive method, e.g., a magnetic resonance method, e.g., MRI or MRS, PET, or SPECT.

In an embodiment, the disorder is, e.g., cancer, said parameter is drug concentration, e.g., at a disorder or disease site, e.g., in a solid tumor. In embodiments the parameter cam be evaluated by a method described herein, e.g., with any of PET-CT, e.g., generally as described in Saleem et al. (2000) The Lancet 355: 2125-2131, MRS, e.g., generally as described in Meisamy et al. (2004) Radiology 233: 424-431, or SPECT, e.g., generally as described in Perik et al. (2006) Journal of Clinical Oncology 24: 2276-2282.

In an embodiment, the disorder is, e.g., cancer, said parameter is blood perfusion, e.g., at a disorder or disease site, e.g., in a solid tumor. In embodiments, the parameter can be evaluated by a method described herein, e.g. MRI, e.g., generally as described in Sorensen et al. (2012) Cancer Research 72: 402-407, or perfusion CT e.g., generally as described in Park et al. (2009) Radiology 250: 110-117, or Doppler ultrasound generally as described in Singh et al. (2010) European J. of Radiology 75: e158-162.

In an embodiment, the disorder is, e.g., cancer, said parameter is oxygenation, e.g., at a disorder or disease site, e.g., in a solid tumor. In embodiments, the parameter can be evaluated by a method described herein, e.g., PET, PET-CT, e.g. generally as described in Rajendran et al. (2006) Clinical Cancer Research 12: 5435-5441, or Eppendorf electrode, e.g. generally described in Le et al. (2007) International J. of Radiation Oncology Biology Physics 69: 167-175, or immunohistochemistry, e.g. generally described in Rademakers et al. (2011) BMC Cancer 11: 167.

In an embodiment, the disorder is, e.g., cancer, said parameter is metabolic activity, e.g., at a disorder or disease site, e.g., in a solid tumor In embodiments the parameter can be evaluated by a method described herein, e.g., functional MRI, or PET, PET-MRI, PET-CT, e.g. generally as described in Shankar et al. (2006) The Journal of Nuclear Medicine 47:1059-1066.

In an embodiment the disorder is, e.g., cancer, said parameter is interstitial fluid pressure, e.g., at a disorder or disease site, e.g., in a solid tumor. In embodiments, the parameter can be evaluated by a method described herein, e.g., the wick-in-needle technique, e.g., generally as described in Boucher et al. (1991) Cancer Research 51: 6691-6694.

In an embodiment, the disorder is a hyperproliferative fibrotic disease and said parameter is amount of connective tissue matrix or blood perfusion.

In an embodiment, the disorder is an inflammatory disorder, said parameter is amount of connective tissue matrix. In embodiments, the parameter can be evaluated immunohistochemically.

In an embodiment, the disorder is an autoimmune disorder, said parameter is amount of connective tissue matrix. In embodiments, the parameter can be evaluated immunohistochemically.

In embodiments, the parameter is evaluated in a sample from said subject, e.g., a tumor sample, e.g., a biopsy, or a blood or serum sample.

In an embodiment, the parameter comprises one or more or all of:

a) drug concentration, e.g., as evaluated by HPLC, or or NMR, e.g., evaluated generally as described in Olive et al. (2009) Science 324: 1475, HPLC with tandem MS, generally as described in Hu et al. (2011) JNCI 103: 893-905, or by histological measures, e.g., fluorescence imaging of fluorescent drugs, generally as described in Primeau et al. (2005) Clinical Cancer Research 11: 8782-8788;

b) collagen content, e.g., as evaluated by total collagen content measured by hydroxyproline content, e.g., generally as described in Netti et al. (2000) Cancer Research 60: 2497-2503, or immunohistochemistry by antibody staining, e.g., generally as described in Pluen et al. (2001) PNAS 98:4628-4633;

c) hyaluronan content, e.g., as evaluated by hyaluronan-binding protein labeling of tissue sections, as generally described in Pluen et al. (2001) PNAS 98:4628-4633, or glycosaminoglycan analysis in tissue extracts, e.g., generally as described in Netti et al. (2000) Cancer Research 60: 2497-2503;

d) pathological response, e.g., the prevalence of tumor cells in a sample, e.g., evaluated generally as described in Minckwitz et al. (2012) Journal of Clinical Oncology published as 10.1200/JCO.2011.38.8595;

e) vessel morphology, e.g., size, can be evaluated generally as described in Provenzano et al. (2012) Cancer Cell 21:418-429, patency (fraction of perfused vessels), e.g., evaluated generally as described in Jacobetz et al. (2012) Gut published on line Mar. 30, 2012, network structure, e.g., evaluated as generally described in Baish et al. (2011) PNAS 108: 1799-1803, luminal opening (measure of perfusion), e.g., evaluated generally as described in Padera et al. (2004) Nature 427: 695, or vessel structure (normalization), e.g., evaluated generally as described in Mazzone et al. (2009) Cell 136:839-851; or

f) hypoxia, e.g., generally as described in Rademakers et al. (2011) BMC Cancer 11: 167 or Le et al. (2007) International J. of Radiation Oncology Biology Physics 69: 167-175. Hypoxia can be evaluated in a number of ways, e.g.: by a pimonizadole method, see, e.g., Kaanders, J. H. et al. (2002) Cancer Res. 62, 7066-7074; an EF5 method, see, e.g., Evans, S. M. et al. (2007) Int. J. Radiat. Oncol. Biol. Phys. 69, 1024-1031; a CA9 method, see, e.g., Koukourakis, M. I. et al., (2006) J. Clin. Oncol. 24, 727-735; a LOX method, see, e.g., Erler, J. T. et al., (2006) Nature 440, 1222-1226; a HIF method, see, e.g., Bos, R. et al. (2003) Cancer 97, 1573-1581, Yan, et al. (2009) Br. J. Cancer 101, 1168-1174, or Koukourakis, M. I. et al., (2006) J. Clin. Oncol. 24, 727-735; or an electrode method, see, e.g., Nordsmark, M. et al. (2005) Radiother. Oncol. 77, 18-24, Brizel, D. M. et al. (1996) Cancer Res. 56, 941-943, Movsas, B. et al. (2002) Urology 60, 634-639, or Fyles, A. et al. (2002) J. Clin. Oncol. 20, 680-687. See generally Table 2 of Wilson and Hays (2011) Nature Rev Cancer, 11: 393-410

In an embodiment, the parameter is evaluated by immunostaining.

In an embodiment, the parameter comprises one or more or all of:

a) serum degraded collagen (ICTP), or collagen synthesis (PIP), e.g., evaluated generally as described in Lopez et al. (2001) Circulation 104:286-291;

b) serum hyaluronan, e.g., evaluated generally as described in Miele et al. (2009) Translational Research 154:194-201; or

c) serum or plasma pro-fibrotic factors (connective tissue growth factor (CTGF), transforming growth factor-beta (TGF-beta), interleukin-1, -4, -6, -8, -10 and -13, platelet-derived growth factor (PDGF), stromal cell-derived factor 1 (SDF1), e.g., evaluated generally as described in Harti et al. (2006) American J. of Respiratory Medicine 173: 1371-1376.

In an embodiment, the parameter is drug concentration and said parameter is evaluated by a chromatographic method, e.g., HPLC.

In an embodiment, the disorder is a hyperproliferative fibrotic disease and the parameter is fibrosis.

In an embodiment, the disorder is an inflammatory disorder and the parameter is fibrosis.

In an embodiment, the disorder is an autoimmune disorder and the parameter is fibrosis.

In an embodiment the parameter is a morphological parameter, e.g., evaluated at a disorder or disease site, e.g., in a solid tumor and comprises one or more or all of:

a) collagen morphology, e.g., evaluated generally as described in Diop-Frimpong et al. (2011) PNAS 108:2909-2914;

b) collagen or hyaluronan content, e.g., evaluated generally as described in Pluen et al. (2001) PNAS 98:4628-4633;

c) vessel patency (fraction of perfused vessels), e.g., evaluated generally as described in Jacobetz et al. (2012) Gut published on line Mar. 30, 2012; or

d) vessel diameter or size evaluated, e.g., evaluated generally as described in Provenzano et al. (2012) Cancer Cell 21:418-429.

In one embodiment, the AHCM is chosen from one or more of: an angiotensin II receptor blocker (AT₁ blocker),

an antagonist of renin angiotensin aldosterone system (“RAAS antagonist”),

an angiotensin converting enzyme (ACE) inhibitor,

a thrombospondin 1 (TSP-1) inhibitor,

a transforming growth factor beta 1 (TGF-β1) inhibitor,

a connective tissue growth factor (CTGF) inhibitor,

a stromal cell-derived growth factor 1 alpha (SDF-1a) inhibitor; or

a combination of two or more of the above.

Unless the context describes otherwise, the term “AHCM” may refer to one or more agents as described herein.

The method can include one, two, three or more AHCMs, alone or in combination with one or more cancer therapies.

In one embodiment, the ARCM is a RAAS antagonist. In an embodiment, the RAAS antagonist is chosen from one or more of: aliskiren (TEKTURNA®, RASILEZ®), remikiren (Ro 42-5892), enalkiren (A-64662), SPP635, or a derivative thereof.

In another embodiment, the AHCM is an AT₁ inhibitor. In an embodiment, the AT₁ blocker is chosen from one or more of: losartan (COZAAR®), candesartan (ATACAND®), eprosartan mesylate (TEVETEN®), EXP 3174, irbesartan (AVAPRO®), L158,809, olmesartan (BENICAR®), saralasin, telmisartin (MICARDIS®), valsartan (DIOVAN®), or a derivative thereof.

In yet another embodiment, the AHCM is an ACE inhibitor. In an embodiment, the ACE inhibitor is chosen from one or more of: benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), or a derivative thereof.

In yet another embodiment, the AHCM is a TSP-1 inhibitor. In an embodiment, the TSP-1 inhibitor is chosen from one or more of: ABT-510, CVX-045, LSKL, or a derivative thereof.

In one embodiment, the AHCM is a TGF-β1 inhibitor, e.g., an anti-TGF-β1 antibody, a TGF-β1 peptide inhibitor. In certain embodiment, the TGF-β1 inhibitor is chosen from one or more of: CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, or a derivative thereof.

In yet another embodiment, the AHCM is a CTGF inhibitor. In certain embodiment, the CTGF inhibitor is chosen from one or more of: DN-9693, FG-3019, and compounds described in European Patent Application Publication No. 1839655, U.S. Pat. No. 7,622,454, or a derivative thereof.

In yet another embodiment, the AHCM is an inhibitor of stromal cell-derived growth factor 1 alpha (SDF-1a/CXCL12a). In certain embodiments, the SDF-1a inhibitor is an anti-SDFla antibody or fragment thereof. In other embodiments, the SDF-1a inhibitor is an inhibitor of an SDF-1a receptor (e.g., a CXCR4 inhibitor), for example Plerixafor (AMD-3100).

The exemplary AHCMs are described herein are not limiting, e.g, derivatives of AHCMs described herein can be used in the methods described herein.

### AHMC Dosage and Dosage Form

Methods of the invention use an AHCM to potentiate a therapy (e.g., a cancer therapy).

In one embodiment, the AHCM is administered at a dose that corresponds to a standard of care dose. Standard of care doses of the AHCM are available in the art. For example, if the AHCM is the AT₁ inhibitor, losartan, the standard of care dose for anti-hypertensive use in a human is about 25-100 mg day⁻¹. In the present methods, losartan can be administered orally in a daily schedule (once or twice a day), alone or in combination with a cancer therapy described herein. Losartan can be provided in a dosage form (e.g., an oral tablet) of about 12.5 mg, 25 mg, 50 mg or 100 mg.

Exemplary standard of care doses for other AT₁ inhibitors for anti-hypertensive or anti-heart failure use in humans are as follows: 4 to 32 mg day⁻¹ of candesartan (ATACAND®) (e.g., available in a dosage form for oral administration containing 4 mg, 8 mg, 16 mg, or 32 mg of candesartan); 400 to 800 mg day⁻¹ of eprosartan mesylate (TEVETEN®) (e.g., available in a dosage form for oral administration containing 400 or 600 mg of eprosartan); 150 to 300 mg day⁻¹ of irbesartan (AVAPRO®) (e.g., available in a dosage form for oral administration containing 150 or 300 mg of irbesartan); 20 to 40 mg day⁻¹ of olmesartan (BENICAR®) (available in a dosage form for oral administration containing 5 mg, 20 mg, or 40 mg of olmesartan); 20 to 80 mg day⁻¹ of telmisartin (MICARDIS®) (e.g., available in a dosage form for oral administration containing of 20 mg, 40 mg or 80 mg of telmisartin); and 80 to 320 mg day⁻¹ of valsartan (DIOVAN®) (e.g., available in a dosage form for oral administration containing 40 mg, 80 mg, 160 mg or 320 mg of valsartan).

In an embodiment, the AHCM is administered at a sub-anti-hypertensive dose (e.g., a dose that has no significant effect on mean arterial blood pressure when administered to a hypertensive subject; or a dose that is below a standard of care anti-hypertensive dose). In an embodiment, the AHCM is administered in an amount that does not substantially lower the mean arterial blood pressure of the subject, e.g., as measured after a pre-selected number of administrations at that dosage, e.g., at the steady state plasma level for a given dosage. In an embodiment, the AHCM is administered, at least once, at a dose that reduces mean arterial blood pressure in the subject by less than 1%, 5%, 10%, 15%, 20%, 25%, 30%, 35%, 40%, 45%, 50%. In an embodiment, the AHCM is administered at a dose that reduces blood pressure by less than 1%, 5%, 10%, 15%, 20%, 25%, 30%, 35%, 40%, 45%, 50%, 60%, 70%, 80%, 90%, or less of the reduction caused by a standard of care anti-hypertensive dose for that AHCM. In an embodiment the AHCM is administered at a dose that is less than 1%, 5%, 10%, 15%, 20%, 25%, 30%, 35%, 40%, 45%, 50%, 60%, 70%, 80%, 90% of the dose of that AHCM that would bring the subject's blood pressure into the normal range, e.g, about 120 systolic and about 80 diastolic, or a dose that would bring the subjects blood pressure into the range of to 120+/−5 systolic and 80+/−5 diastolic.

In an embodiment, the AHCM is administered at a dose that is less than the standard of care dose for anti-hypertensive or anti-heart failure use (e.g., a dose that is less than 0.01, 0.02, 0.03, 0.04, 0.05, 0.06, 0.07, 0.08, 0.09, 0.1, 0.2, 0.3, 0.4, 0.5, 0.6, 0.7, that of the standard of care dose for anti-hypertensive or anti-heart failure use). Standard of care doses of the AHCM are available in the art. For example, if the AHCM is the AT₁ inhibitor, losartan, and the standard of care dose is about 25-100 mg day', the suboptimal anti-hypertensive drug can range from 0.25 to 17.5, 0.5 to 15, 1.3 to 12, 1.5 to 12, 2 to 12, 2 to 10, 2 to 5, 2 to 3 mg day⁻¹, typically, 2 mg day⁻¹. In one embodiment, the AHCM is losartan and is administered at a dose less than 25, 20, 15, 10, 5, 4, 3, 2, 1 mg day⁻¹. Losartan can be administered orally in a daily schedule (once or twice a day) at a sub-anti-hypertensive dose of 2-3 mg day⁻¹, alone or in combination with a cancer therapeutic described herein. Exemplary standard of care doses for other AT₁ inhibitors are as follows: 4 to 32 mg day⁻¹ of candesartan (ATACAND®), 400 to 800 mg day⁻¹ of eprosartan mesylate (TEVETEN®), 150 to 300 mg day⁻¹ of irbesartan (AVAPRO®), 20 to 40 mg day⁻¹ of olmesartan (BENICAR®), 20 to 80 mg day⁻¹ of telmisartin (MICARDIS®), and 80 to 320 mg day⁻¹ of valsartan (DIOVAN®). In an embodiment, the AHCM is administered at a dose that is less than the standard of care dose of the anti-hypertensive or anti-heart failure dose (e.g., a dose that is less than 0.01, 0.02, 0.03, 0.04, 0.05, 0.06, 0.07, 0.08, 0.09, 0.1, 0.2, 0.3, 0.4, 0.5, 0.6, 0.7, that of the standard of care dose of the anti-hypertensive or anti-heart failure dose for other AT₁ inhibitors such as candesartan, eprosartan, irbesartan, olmesartan, telmisartin, and valsartan). In certain embodiments, the AHCM is formulated in a dosage form that is less than the standard of care anti-hypertensive or anti-heart failure dosage form (e.g., a dosage form that is less than 0.01, 0.02, 0.03, 0.04, 0.05, 0.06, 0.07, 0.08, 0.09, 0.1, 0.15, 0.16, 0.2, 0.3, 0.4, 0.5, 0.6, 0.7, that of the standard of care dosage form). For example, if the AHCM is losartan, the dosage form can be of about 0.5 mg-11 mg; 1 mg-10 mg; 1-5 mg, or 1 mg, 2 mg, 3 mg, 4 mg, 5 mg, 6 mg, 7 mg, 8 mg, 9 mg or 10 mg. In some embodiments, losartan can be provided in a dosage form (e.g., an oral tablet) below 12.5 mg, e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, or about 12 mg.

In one embodiment, the ARCM is formulated and/or dosed for oral administration. In one embodiment, the AHCM is formulated as a tablet (e.g., an oral tablet). In other embodiments, the AHCM is formulated and/or dosed for other routes of administration, e.g., subcutaneous, intravenous, or intraperitoneal administration. In certain embodiments, the AHCM can be formulated and/or dosed for extended, delayed, or controlled release, e.g., in an extended release formulation (e.g., an oral formulation) for substantially continuous release over a period of hours (e.g., at least 1, 2, 3, 4, 5, 10, or 24 hours); days (e.g., at least 1, 2, 5, 10, 14 days, or longer), weeks, months or years.

In some embodiments, the sub-anti-hypertensive dose of the AHCM or a dose of the AHCM that is less than the standard of care dose for anti-hypertensive or anti-heart failure use can be a dose that is insufficient to inhibit or prevent tumor growth or progression if it is administed to a subject by itself.

In yet another embodiment, the AHCM is administered at a dose that is greater than the standard of care dose for anti-hypertensive or anti-heart failure use (e.g., a dose that is greater than 1.1, 1.5, 1.7, 2, 3, 4, 5, 10-fold or higher, that of the standard of care dose for anti-hypertensive or anti-heart failure use). Standard of care doses of the AHCM are available in the art; some of which are exemplified herein.

In other embodiments, the AHCM is formulated in a dosage form that is greater than the standard of care anti-hypertensive or anti-heart failure dosage form (e.g., a dosage form that is greater than 1.1, 1.5, 1.7, 2, 3, 4, 5, 10-fold or higher, that of the standard of care dosage form). Standard of care dosage forms of the AHCM are available in the art; some of which are exemplified herein.

In some embodiments, a dose of the AHCM that is comparable to, or greater than the standard of care anti-hypertensive or anti-heart failure dose can be a dose that is insufficient to inhibit or prevent tumor growth or progression if it is administered to a subject by itself.

In other embodiments, the anti-cancer agent is administered at a greater dosage, or in a regimen that results in higher levels of the anti-cancer agent, as compared with a reference, e.g., the dosage on a package insert, the standard of care dosing, or the maximum tolerated dose (MTD).

In certain embodiments, the anti-cancer agent is administered at a lesser dosage, or in a regimen that results in lower levels of the anti-cancer agent, as compared with a reference, e.g., the dosage on a package insert, the standard of care dosing, or the MTD. In some embodiments, the anti-cancer agent is administered in an amount such that it is not effective to inhibit or prevent tumor growth or progression when administered by itself, but in an amount sufficient to inhibit or prevent tumor growth or progression when administered in combination with the AHCM.

In some embodiments, the cancer therapy or cancer therapeutic, when administered in combination with an AHCM, is administered to the subject at a dose that is less than the lowest dose that would be used in the absence of the AHCM, to treat or prevent cancer in a subject.

In some embodiments, when both the ARCM agent and cancer therapy or cancer therapeutic are administered to the subject, the dose of the anti-hypertensive and/or collagen modifying agent can be a dose that is less than the lowest dose that would be used to treat a hypertensive-associated disorder or heart failure, while the dose of the cancer therapy or cancer therapeutic can be a dose that is less than the lowest dose that would be used in the absence of the AHCM, to treat or prevent cancer in a subject.

In some embodiments, while the dose of the cancer therapy or cancer therapeutic administered to the subject is less than the lowest dose that would be used alone to treat a patient with cancer, the dose of the AHCM agent administered to the subject as an adjuvant can be less than the lowest dose that would be used alone to treat cancer. In such embodiments, the dose of the AHCM agent administered to the subject as an adjuvant can be sub-anti-hypertensive dose or comparable to, or greater than the standard care dose for treatment of hypertension or heart failure.

In some embodiments, while the dose of the cancer therapy or cancer therapeutic administered to the subject is less than the lowest dose that would be used in the absence of the AHCM, to treat a patient with cancer, the dose of the AHCM agent administered to the subject as an adjuvant can be less than the lowest dose that would be used alone to treat cancer, but is sufficient to improve efficacy of a cancer therapy or delivery of a cancer therapeutic to a tumor. In such embodiments, the dose of the AHCM agent administered to the subject as an adjuvant can be sub-anti-hypertensive dose or comparable to, or greater than the standard care dose for treatment of hypertension or heart failure.

Methods to determine the lowest dose of any agent, e.g., an anti-cancer agent and/or an AHCM, for treatment are well known within one of skill in the art. For example, a skilled artisan can determine the lowest dose of an AHCM and/or an anti-cancer agent effective for treatment in an animal model corresponding to a specific type of cancer, e.g., by administering the animal with different doses of the AHCM and/or anti-cancer agent and monitoring the tumor growth as compared to a control. A control can be an animal treated with an anti-cancer agent alone (i.e., in the absence of the AHCM).

The AHCM and the therapy (e.g., cancer therapy) can be administered in combination, e.g., sequentially and/or concurrently, as described herein. The AHCM and the therapy can be administered (at the same or different dosages) in any order and/or overlap with the therapy. In one embodiment, the AHCM is administered before the therapy. In other embodiments, the AHCM is administered sequentially and/or concurrently with the therapy (e.g., the AHCM is administered prior to the therapy and concurrently with the therapy). In yet other embodiments, the cancer therapy is administered first, and the AHCM is administered after initiation of the cancer therapy, or is administered after cessation of the therapy. In other embodiments, the administration of the AHCM starts after cessation of the therapy (e.g., with or without a gap between the cessation of the therapy and the beginning of the AHCM). In other embodiments, the administration of the AHCM continues after cessation of the therapy. In embodiments where administration of the AHCM and therapy is concurrent, the administration of the AHCM and the cancer therapy can be continued as clinically appropriate (i) as a combination therapy, (ii) with a period of therapy with either the AHCM or the cancer therapy, or (iii) a combination of (i) and (ii) in any order.

The administration of the AHCM can be substantially continuous. For example, administration of the AHCM can be substantially continuously over a period of at least 1, 5, 10, 24 hours; 2, 5, 10, 14 days, or longer. As described in the Examples herein, substantially continuous administration of an AHCM (e.g., via a subcutaneous pump) causes a greater reduction in collagen content and/or tumor size than single or pulsatile administration (e.g., single or multiple subcutaneous administrations) of the AHCM.

In some embodiments, the AHCM administration continues after the therapy has ceased, e.g., over a period of hours, days, months or years.

In other embodiments, the administration of the AHCM can be intermittent, e.g., can have gaps at pre-determined intervals, during the course of therapy. In certain embodiments, two or more doses of the AHCM are administered, alone or in combination with the therapy (e.g., the cancer therapy). In one embodiment, the AHCM is administered at a suboptimal anti-hypertensive dose and an anti-hypertensive dose during the course of therapy. For example, a suboptimal anti-hypertensive dose of the AHCM can be administered prior to, or at the time, of therapy (e.g., cancer therapy) (e.g., treatment with an anti-cancer agent that increases mean arterial blood pressure, e.g, treatment with an anti-angiogenic drug (e.g., Avastin, sunitinib or sorafenib)); then followed by a second hypertensive dose of the AHCM.

### Size of Therapeutic Entities

The methods described herein allow for enhanced flexibility in the range of treatment modalities used or selected, e.g., in the size of the therapeutic entity or entities._Accordingly, in one embodiment, an ARCM is administered as an entity having a hydrodynamic diameter of greater thanabout 1, 5, 10, 100, 500, or 1,000 nm. E.g., the AHCM can be a protein, e.g., an antibody. The AHCM can also be administered as a nanoparticle, e.g., a polymeric nanoparticle or a liposome, that includes the AHCM as a small molecule therapeutic or a protein, e.g., an antibody.

In an embodiment, the therapy is a cancer therapeutic (also referred to herein as “an anti-cancer agent”) or second therapeutic agent is administered as an entity having a hydrodynamic diameter of greater than about 1, 5, 10, 20, 50, 75, 100, 150, 200, 500, or 1,000 nm. E.g., the second therapeutic agent (e.g., the anti-cancer agent) can be a protein, e.g., an antibody. The second therapeutic agent (e.g., the anti-cancer agent) can also be administered as a nanoparticle, e.g., a polymeric nanoparticle or a liposome, that includes the agent as a small molecule therapeutic (i.e., a molecule having a hydrodynamic diameter of about 1 nm or less) or a protein, e.g., an antibody.

In an embodiment, an AHCM is administered as an entity having a hydrodynamic diameter of greater than about 1 nm (e.g., greater than about 1, 5, 10, 20, 50, 75, 100, 150, 200, 500, or 1,000 nm) and a second therapeutic agent (e.g., an anti-cancer agent) is administered as an entity having a hydrodynamic diameter of about 1 nm or less. In one embodiment, the AHCM is present in the entity without a second therapeutic agent (e.g., a chemotherapeutic agent). The AHCM can be formulated for extended release, e.g., in an extended release formulation for substantially continuous release for hours, days, weeks, months or years.

In an embodiment, an AHCM is administered as an entity having a hydrodynamic diameter of about 1 nm, or less, and a second therapeutic agent (e.g., an anti-cancer agent) is administered as an entity having a hydrodynamic diameter of about 1 nm or greater (e.g., greater than about 1, 5, 10, 20, 50, 75, 100, 150, 200, 500, or 1,000 nm).

In an embodiment, an AHCM is administered as an entity having a hydrodynamic diameter of less than, or equal to, about 1 nm and a second therapeutic agent (e.g., an anti-cancer agent) is administered as an entity having a hydrodynamic diameter of less than about 1 nm.

In an embodiment, an AHCM is administered as an entity having a hydrodynamic diameter of greater than about 1 nm (e.g., greater than about 1, 5, 10, 20, 50, 75, 100, 150, 200, 500, or 1,000 nm), and a second therapeutic agent (e.g., an anti-cancer agent) is administered as an entity having a hydrodynamic diameter of greater than about 1 nm (e.g., greater than about 1, 5, 10, 20, 50, 75, 100, 150, 200, 500, or 1,000 nm). The AHCM and the second therapeutic agent (e.g., the anti-cancer agent) can be in separate or the same entity. For example, if provided as separate entities the AHCM can be provided as a first nanoparticle and the second therapeutic agent (e.g., the anti-cancer agent) provided as a second nanoparticle (e.g., where the second nanoparticle has a structural property (e.g., size or composition) or a functional property (e.g., release kinetics or a pharmacodynamic property) that differs from the first nanoparticle). Alternatively, an AHCM and a second therapeutic agent (e.g., an anti-cancer agent) can be provided on the same entity, e.g., in the same nanoparticle.

In an embodiment, the AHCM is selected from a therapeutic entity having a hydrodynamic diameter: equal to or less than 1 or 2 nm; between 2-20, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300, 1000 nm; or 10, 15, 20, 25, 35, 45, 50, 75, 100, 150 or 200 nm.

In an embodiment, the AHCM: is a small molecule therapeutic; is a protein, e.g., an antibody; or is provided in a nanoparticle.

In an embodiment, the anti-cancer agent or second therapeutic agent is selected from a therapeutic entity having a hydrodynamic diameter: equal to or less than 1 or 2 nm; between 2-20, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300-1000 nm; or 10, 15, 20, 25, 35, 45, 50, 75, 100, 150 or 200 nm.

In an embodiment, the anti-cancer agent or second therapeutic agent: is a small molecule therapeutic with a hydrodynamic diameter of 1 nm or less; is a protein, e.g., an antibody; or is provided in a nanoparticle.

In an embodiment, the AHCM, or anti-cancer agent or the second therapeutic agent, each independently, can be provided as an entity having the following size ranges (in nm): a hydrodynamic diameter of less than or equal to 1, or between 0.1 and 1.0 nm, e.g., that of a typical small molecule; a hydrodynamic diameter of between 5 and 20, or 5 and 15 nm, e.g., that of a protein, e.g., an antibody; or a hydrodynamic diameter of 10-5,000, 20-1,000, 10-500, 10-200, 10-150, or 10-100, 10-25, 20-40, 40, 50-150 nm; between 10, 15, 20, 25, 35, 40, 45, 50-100 nm; between 10, 15, 20, 25, 35, 40, 45, 50-200 nm; between 10, 15, 20, 25, 35, 40, 45, 50, 75, 100, 150, 200, 300-500 nm; and between 10, 15, 20, 25, 35, 40, 45, 50, 75, 100, 150, 200, 300-1000 nm; or 10, 15, 20, 25, 35, 45, 50, 75, 100, 150 or 200 nm, e.g., a range of typical nanoparticles.

### Subjects

Methods described herein can be used to treat subjects having characteristics or needs defined herein. In embodiments a subject, or a treatment for a subject, is selected on the basis of a characteristic described herein. In one embodiment, the methods described herein allow optimized selection of patients and therapies.

In some embodiments, subjects can be selected or identified prior to subjecting them to any aspects of the methods described herein.

In one embodiment, the subject is selected or is identified as being in need of receiving the AHCM and/or the microenvironment modulator on the basis of optimizing a therapy, e.g., the need for improved delivery and/or efficacy of the therapy (e.g., the cancer therapy).

In one embodiment, the subject does not have hypertension, or is not being treated for hypertension, at the time of initiation of the AHCM treatment, or at the time of selection of the patient for AHCM administration.

In an embodiment, the subject, e.g., patient, has not been administered a dose of an AHCM, e.g., an AHCM named herein, or any AHCM, within 5, 10, 30, 60 or 100 days of, the diagnosis of cancer, or the initiation of the AHCM dosing.

In an embodiment, the subject, e.g., a subject with normal or low blood pressure, is selected or is identified on the basis of being in need of an AHCM and/or the microenvironment modulator, e.g., is selected or is identified as being in need of receiving the AHCM and/or the microenvironment modulator on the basis of optimizing a therapy, e.g., the need for improved delivery and/or efficacy of the therapy (e.g., the cancer therapy).

In some embodiments, subjects who are in need of receiving the AHCM and/or the microenvironment modulator on the basis of the need for improved delivery or efficacy of the cancer therapy, or optimizing the therapy, are the subjects who partially respond or do not respond to the cancer therapy alone.

In an embodiment, an AHCM and/or the microenvironment modulator is selected for treating a subject, on the basis of its ability to optimize a treatment, e.g., a cancer treatment, e.g., improving delivery and/or efficacy of the therapy, e.g., the cancer therapy.

In an embodiment, the subject treated is not a hypertensive patient, e.g., does not have a medical history of high blood pressure, or has not been treated with an anti-hypertensive agent. In one embodiment, the subject treated has normal or low mean arterial blood pressure. In other embodiments, the subject treated has not undergone, or is not being treated with anti-hypertensive therapy.

In certain embodiments, the subject has a disorder chosen from one or more of a hyperproliferative disorder, a cancer, a fibrotic disorder, an inflammatory disorder or an autoimmune disorder.

In one embodiment, the subject is in need of cancer therapy. In another embodiment, the subject is in need of, or being considered for, anti-cancer therapy (e.g., treatment with any of the anti-cancer therapeutics described herein). In certain embodiments, the method includes the step of determining if the subject has a cancer (e.g., a solid or fibrotic cancer), and, responsive to said determination, administering the AHCM and/or the microenvironment modulator, and the anti-cancer agent.

In other embodiments, the subject is at risk of developing, or having a recurrence of, a cancer, e.g., a subject with pre-neoplasia or a genetic pre-disposition for cancer (e.g., a subject having a BRCA1 mutation; or a breast cancer patient treated with in an adjuvant setting (e.g., with tamoxifen)).

In other embodiments, the subject has early-cancer, or more progressive (e.g., moderate), or metastatic cancer.

In one embodiment, the subject has a solid, fibrotic tumor chosen from one or more of pancreatic (e.g., pancreatic adenocarcinoma or pancreatic ductal adenocarcinoma), breast, colorectal, colon, lung (e.g., small or non-small cell lung cancer), skin, ovarian, prostate, cervix, gastrointestinal (e.g., carcinoid or stromal), stomach, head and neck, kidney, or liver cancer, or a metastatic lesion thereof. Additional examples of cancers treated are described herein below.

In one embodiment, the subject has a fibrotic or desmoplastic solid tumor, e.g., a tumor having one or more of: limited tumor perfusion, compressed blood vessels, high interstitial fluid pressure (IFPs), or fibrotic tumor interstitium. In certain embodiments, the subject has a tumor having (e.g., elevated levels of) extracellular matrix components, such as fibers (e.g., collagen, procollagen) and/or polysaccharides (e.g., glycosaminoglycans such as hyaluronan or hyaluronic acid). The levels of the extracellular matrix components in the tumor can vary depending on the particular cancer type, the stage of maligancy, and/or in response to cancer therapy. For example, certain tumors may show elevated levels of extracellular matrix components in response to chemotherapy and/or radiation. In such cancers, the AHCM alone or in combination with the microenvironment modulator can be administered at any time before, during or after the cancer therapy.

In other embodiments, the subject has a hyperproliferative cancerous condition (e.g., a benign, pre-malignant or malignant condition). The subject can be one at risk of having the disorder, e.g., a subject having a relative afflicted with the disorder, or a subject having a genetic trait associated with risk for the disorder. In one embodiment, the subject can be symptomatic or asymptomatic. In an embodiment, the subject harbors an alteration in an oncogenic gene or gene product. In an embodiment, the subject is a patient who is undergoing cancer therapy (e.g., the same or other anti-cancer agents, surgery and/or radiation). In an embodiment, the subject is a patient who has undergone cancer therapy (e.g., other anti-cancer agents, surgery and/or radiation). In one embodiment, the subject has not been treated with the cancer therapy.

In one embodiment, the subject is a patient with a metastatic cancer, e.g., a metastastatic form of a cancer disclosed herein (one or more of pancreatic (e.g., pancreatic adenocarcinoma), breast, colorectal, lung (e.g., small or non-small cell lung cancer), skin, ovarian, or liver cancer.

In one embodiment, the subject is a patient having treatment-resistant cancer or hyperproliferative disorder.

In some embodiments, the subject being selected for subjecting to the methods or pharmaceutical compositions herein does not have a renal disease or a disease associated with kidneys.

In one embodiment, the subject treated is a mammal, e.g., a primate, typically a human (e.g., a patient having, or at risk of, a cancer or tumor as described herein).

In certain embodiments, the subject treated has a disorder chosen from one or more of a hyperproliferative disorder, a cancer, a fibrotic disorder, an inflammatory disorder or an autoimmune disorder.

In one embodiment, the subject treated has a hyperproliferative disorder, e.g., a hyperpoliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the lung, liver, heart, kidney, pancreas, skin and other organs).

In other embodiment, the subject treated has a hyperproliferative genetic disorder, e.g., a hyperproliferative genetic disorder chosen from Marfan's syndrome or Loeys-Dietz syndrome.

In other embodiments, the hyperproliferative disorder (e.g., the hyperproliferative fibrotic disorder) is chosen from one or more of chronic obstructive pulmonary disease, asthma, aortic aneurysm, radiation-induced fibrosis, skeletal-muscle myopathy, diabetic nephropathy, and/or arthritis.

### Combination Therapies

In one embodiment, the AHCM is administered in combination with a microenvironment modulator, and/or a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the AHCM and the therapy, e.g., the cancer therapy, can be sequential (with or without overlap) or simultaneous. Administration of the AHCM and/or the microenvironment modulator can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

In an embodiment, administration of the AHCM and/or the microenvironment modulator is initiated prior to the initiation of administration of the therapy (e.g., the cancer therapy), e.g., it is initiated at least one, two, three, or five days, or one, two, three, four, five or more weeks prior to cancer therapy (e.g., the AHCM and/or the microenvironment modulator is administered at a minimum of two weeks prior to cancer therapy). In an embodiment, it is initiated no more than 5, 10, 20, 30, 60 or 120 days prior to initiation of the therapy, e.g., the cancer therapy. In an embodiment, administration of the AHCM and/or the microenvironment modulator is initiated prior to the therapy, e.g., the cancer therapy, and the therapy is not initiated until a criterion is met, e.g., a time-based criterion, e.g., administration of AHCM and/or the microenvironment modulator for a predetermined number of days or for a predetermined number of administrations. In an embodiment, the criterion is meeting a preselected level of AHCM and/or the microenvironment modulator, e.g., a preselected level in serum, plasma or tissue. In one embodiment, the criterion is meeting a preselected level of a biomarker in plasma, serum or tissue, including but not limited to, an angiotensin receptor (e.g., angiotension-II type-1 receptor; AT_{1A} receptor (AT_{1A}R)), collagen I, collagen III, collagen IV, transforming growth factor beta 1 (TGF-β1), connective tissue growth factor (CTGF), or thrombospondin-1 (TSP-1). In another embodiment, the criterion is meeting a preselected level of alteration in tumor morphology.

In one embodiment, the administration of the AHCM and/or the microenvironment modulator is sequential and/or concurrent with the therapy, e.g., the cancer therapy, as described herein.

In an embodiment, the AHCM and/or the microenvironment modulator is administered, or a preselected level, e.g., a plasma level, of AHCM and/or the microenvironment modulator is maintained for a preselected portion of the time the subject receives the therapy, e.g., the cancer therapy. By way of example, the AHCM and/or the microenvironment modulator therapy is maintained for the entire period in which the therapy, e.g., the cancer therapy, is administered, or for the entire period in which a preselected level of the therapy (e.g., an anti-cancer agent) persists in the subject.

Typically, therapy with the AHCM and/or the microenvironment modulator continues during the entire therapy, e.g., cancer therapy, schedule. In yet other embodiments, administration of the AHCM and/or the microenvironment modulator is discontinued prior to cessation of the therapy, e.g., the cancer therapy. In other embodiments, administration of the AHCM and/or the microenvironment modulator is continued after cessation of the therapy, e.g., the cancer therapy, e.g., the administration continues hours, days, months or more, after cessation of the cancer therapy.

In an embodiment, two or more doses of the AHCM and/or the microenvironment modulator are administered, alone or in combination with the therapy, e.g., the cancer therapy. In one embodiment, the AHCM is administered at a sub-anti-hypertensive dose and an anti-hypertensive dose during the course of therapy. For example, a sub-anti-hypertensive dose of the AHCM can be administered prior to, or at the time, of the therapy, e.g., the cancer therapy (e.g., treatment with an anti-cancer agent that increases mean arterial blood pressure, e.g., treatment with an anti-angiogenic drug (e.g., Avastin, sunitinib or sorafenib)); then followed by a subsequent hypertensive dose of the AHCM.

In one embodiment, the ARCM (alone or in combination) is administered substantially continuously over a period of, or at least 15, 30, 45 minutes; a period of, or at least, 1, 5, 10, 24 hours; a period of, or at least, 2, 5, 10, 14 days; a period of, or at least, 3, 4, 5, 6, 7, 8 weeks; a period of, or at least 2, 3, 4, 5, 6, 7, 8, 9, 10, 11 months; a period of, or at least, 1, 2, 3, 4, 5 years, or longer. In one embodiment, the AHCM is administered as a controlled- or sustained release formulation, dosage form, or device. In certain embodiments, the AHCM is formulated for continuous delivery, e.g., oral, subcutaneous or intravenous continuous delivery. In one embodiment, the AHCM (alone or in combination with the microenvironment modulator and/or cancer therapy) is in an oral controlled- or extended release dosage form or formulation. In one embodiment, the AHCM is administered via an implantable device, e.g., a pump (e.g., a subcutaneous pump), an implant or a depot. The delivery method can be optimized such that an AHCM dose as described herein (e.g., a standard, sub-hypertensive, or higher than standard dose) is administered and/or maintained in the subject for a pre-determined period (e.g., a period of, or at least: 15, 30, 45 minutes; 1, 5, 10, 24 hours 2, 5, 10, 14 days; 3, 4, 5, 6, 7, 8 weeks; 2, 3, 4, 5, 6, 7, 8, 9, 10, 11 months; 1, 2, 3, 4, 5 years, or longer). The substantially continuously or extended release delivery or formulation of the AHCM (with or without the combination of the microenvironment modulator and/or therapy) can be used for prevention or treatment of cancer for a period of hours, days, weeks, months or years. In one embodiment, the therapy is chosen from one or more of: nanotherapy (e.g., a viral cancer therapeutic agent (e.g., an oncolytic herpes simplex virus (HSV), a lipid nanoparticle (e.g., a liposomal formulation (e.g., pegylated liposomal doxorubicin (DOXIL®)), or a polymeric nanoparticle); an antibody that binds to a cancer target; an RNAi or antisense RNA agent; a chemotherapeutic agent (e.g., a cytotoxic or a cytostatic agent); PDT, immunotherapy, radiation; or surgery; or any combination thereof. Additional examples of anti-cancer therapies that can be used in combination with the AHCM are provided below.

In other embodiments, the AHCM and the therapy (e.g., the cancer or hyperproliferative therapy) are administered to a subject, e.g., a subject as described herein, in combination with the microenvironment modulator. In certain embodiments, the microenvironment modulator causes one or more of: reduces solid stress (e.g., growth-induced solid stress in tumors); decreases tumor fibrosis; reduces interstitial hypertension or interstitial fluid pressure (IFP); increases interstitial tumor transport; increases tumor or vessel perfusion; increases vascular diameters and/or enlarges compressed or collapsed blood vessels; reduces or depletes one or more of: cancer cells, or stromal cells (e.g., tumor associated fibroblasts or immune cells); decreases the level or production of extracellular matrix components, such as fibers (e.g., collagen, procollagen), and/or polysaccharides (e.g., glycosaminoglycans such as hyaluronan or hyaluronic acid); decreases the level or production of collagen or procollagen; decreases the level or production of hyaluronic acid; increases tumor oxygenation; decreases tumor hypoxia; decreases tumor acidosis; enables immune cell infiltration; decreases immunosuppression; increases antitumor immunity; decreases the production of cancer stem cells (also referred to herein as tumor-initiating cells); or enhances the efficacy (e.g., penetration or diffusion), of the therapy, e.g., the cancer therapy (e.g., radiation, photodynamic therapy, chemotherapeutics and immunotherapies) in a tumor or tumor vasculature, in the subject.

In one embodiment, the microenvironment modulator includes an anti-angiogenic therapy, for example, an inhibitor of vascular endothelial growth factor (VEGF) pathway. Exemplary VEGF pathway inhibitors include, but are not limited to, an antibody against VEGF (e.g., bevacizumab); a VEGF receptor inhibitor (e.g., an inhibitor of VEGFR-1 inhibitor, a VEGFR-2 inhibitor, or a VEGFR-3 inhibitor (e.g., VEGFR inhibitors such as Cediranib (AZD2171)); a VEGF trap (e.g., a fusion protein that includes a VEGFR domain (e.g., a VEGFR1 domain 2 and a VEGFR2 domain 3) fused to an Fc fragment of an IgG); and an anti-VEGF aptamer (or a pegylated derivative thereof (e.g., MACUGEN®).

In another embodiment, the microenvironment modulator includes an agent that decreases the level or production of hyaluronic acid, including but not limited to, an antibody against hyaluronic acid, and an anti-hyaluronan enzymatic therapy, such as hyaluronidase or a derivative thereof (e.g., pegylated form thereof) (e.g., PH20, or pegylated, recombinant human hyaluronidase PEGPH20).

In another embodiment, the microenvironment modulator includes an inhibitor of the hedgehog pathway, e.g., IPI-926, GDC-0449, cylopamine or an analogue thereof, or GANT58.

In another embodiment, the microenvironment modulator includes an agent that improves drug penetration in tumors. In one embodiment, the agent is a disulfide-based cyclic RGD peptide peptide (iRGD) or an analogue thereof.

In yet another embodiment, the microenvironment modulator includes a taxane therapy (e.g., taxane-induced apoptosis as described in Griffon-Etienne, G. et al. (1999) Cancer Res. 59(15):3776-82).

In another embodiment, the microenvironment modulator includes an agent that modulates (e.g, inhibits) a hypoxia inducible factor (HIF), for example, an agent that inhibits hypoxia-inducible factors 1α and 2α (HIF-1α and HIF-2α). In one embodiment, the agent is an antibody against an HIF. In another embodiment, the agent is an HIF chemical inhibitor, such as phenethyl isothiocyanate (PEITC).

In another embodiment, the microenvironment modulator includes an agent that decreases the level or production of collagen or procollagen. For example, an agent that degrades collagen, e.g., collagenase.

In yet another embodiment, the microenvironment modulator is an anti-fibrotic agent or inhibitor of a profibrotic pathway (a “profibrotic pathway inhibitor”) (e.g., a pathway dependent- or independent of TGF-beta and/or CTGF activation). In one embodiment, the AHCM and/or the cancer therapy is administered in combination with one or more of: an inhibitor of endothelin-1, PDGF, Wnt/beta-catenin, IGF-1, TNF-alpha, and/or IL-4. In another embodiment, the AHCM and/or the cancer therapy is administered in combination with an inhibitor of endothelin-1 and/or PDGF. In other embodiments, the AHCM and/or the cancer therapy is administered in combination with an inhibitor of one or more of: chemokine receptor type 4 (CXCR4) (e.g., AMD3100, MSX-122); stromal-derived-factor-1 (SDF-1) (e.g., tannic acid); hedgehog (e.g., IPI-926, GDC-0449, cylopamine or an analogue thereof, or GANT58).

In another embodiment, the AHCM and/or the cancer therapy is administered in combination with an anti-fibrotic agent, for example, a pirfenidone (PFD, 5-methyl-1-phenyl-2-(1H)-pyridone), as further described herein.

The administration of the AHCM, the cancer therapy, the microenvironment modulator and/or the profibrotic pathway inhibitor can be sequential (with or without overlap) or simultaneous (e.g., a described herein).

### Cancer Therapies

In one embodiment, the cancer treated is an epithelial, mesenchymal or hematologic malignancy. In an embodiment, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, high interstitial fluid pressure (IFPs), or fibrotic tumor interstitium. In one embodiment, the solid tumor is chosen from one or more of pancreatic (e.g., pancreatic adenocarcinoma (e.g., pancreatic ductal adenocarcinoma (PDA)), breast, gastric, colorectal, lung (e.g., small or non-small cell lung cancer), skin, ovarian, prostate, or liver cancer. Additional examples of cancers treated are described herein below.

In certain embodiments, the cancer treated contains (e.g., has elevated levels of) extracellular matrix components, such as fibers (e.g., collagen, procollagen) and/or polysaccharides (e.g., glycosaminoglycans such as hyaluronan or hyaluronic acid). The levels of the extracellular matrix components in the cancer can vary depending on the particular cancer type, the stage of malignancy, and/or in response to cancer therapy. For example, certain cancer may show elevated levels of extracellular matrix components in response to chemotherapy and/or radiation. In such cancers, the AHCM alone or in combination with the microenvironment modulator can be administered at any time before, during or after the cancer therapy.

In another embodiment, the AHCM and/or the microenvironment modulator is administered in combination with a cancer therapy (e.g., one or more of anti-cancer agents, photodynamic therapy (PDT), immunotherapy, surgery and/or radiation). In one embodiment, the cancer therapy includes one or more of: a cancer therapeutic, including, for example, a nanotherapy (e.g., one or more nanotherapeutic agents, including viral cancer therapeutic agents (e.g., an oncolytic herpes simplex virus (HSV)) a lipid nanoparticle (e.g., a liposomal formulation (e.g., pegylated liposomal doxorubicin (DOXIL®)), or a polymeric nanoparticle); one or more cancer therapeutic antibodies (e.g., anti-HER2, anti-EGFR, anti-CD20 antibodies); RNAi and antisense RNA agents; one or more chemotherapeutic agents (e.g., low molecular weight chemotherapeutic agents, including a cytotoxic or a cytostatic agent)); photodynamic therapy; immunotherapy; radiation; or surgery, or any combination thereof. Any combination of one or more AHCMs and one or more therapeutic modalities (e.g., first, second, third) nanotherapeutic agent, antibody agent, low molecular weight chemotherapeutic agent, radiation can be used. Exemplary cancer therapeutics include, but are not limited to, nanotherapeutic agents (e.g., one or more lipid nanoparticles (e.g., a liposomal formulation (e.g., pegylated liposomal doxorubicin (DOXIL®) or liposomal paclitaxel (e.g., Abraxane®)), or a polymeric nanoparticle); one or more low molecular weight chemotherapeutics (e.g., gemcitabine, cisplatin, epirubicin, 5-fluorouracil, paclitaxel, oxaliplatin, or leucovorin); one or more antibodies against cancer targets (e.g., growth factor receptor such as HER-2/neu, HER3, VEGF)); one or more tyrosine kinase inhibitors, e.g., including low molecular weight and antibody agents, such as sunitinib, erlotinib, gefitinib, sorafenib, icotinib, lapatinib, neratinib, vandetanib, BIBW 2992 or XL-647, anti-EGFR antibody (e.g., cetuximab, panitumumab, zalutumumab, nimotuzumab necitumumab or matuzumab)). Additional examples of chemotherapeutic agents used in combination therapies are described hereinbelow.

In one embodiment, the chemotherapeutic agent used in combination with the AHCM and/or the microenvironment modulator is a cytotoxic or a cytostatic agent. Exemplary cytotoxic agents include antimicrotubule agents, topoisomerase inhibitors (e.g., irinotecan), or taxanes (e.g., docetaxel), antimetabolites, mitotic inhibitors, alkylating agents, intercalating agents, agents capable of interfering with a signal transduction pathway, agents that promote apoptosis and radiation. In yet other embodiments, the methods can be used in combination with immunodulatory agents, e.g., IL-1, 2, 4, 6, or 12, or interferon alpha or gamma, or immune cell growth factors such as GM-CSF.

In other embodiments, the cancer therapy includes an immune or immunotherapy used in combination with the AHCM, other cancer therapies, and/or the microenvironment modulator, described herein. Without wishing to be bound by theory, factor such as hypoxia and/or limited perfusion are believed to cause immunosuppression and/or limit the efficacy of certain immune therapies. AHCM, alone or in combination with therapies described herein, can be used to improve the efficacy of said immune therapies. Examples of immune therapies include, but are not limited to, CTLA-4 blockade (e.g., an anti-CTLA-4 antibody (e.g., ipilimumab)); immune-based therapies (including, e.g., immune or dendritic cell-based vaccines and antagonists of immune inhibitory signals or checkpoints); cancer vaccines, e.g., Sipuleucel-T (APC8015, trade name Provenge); and adoptive T-cell-based therapies. Exemplary immune-based therapies include, but are not limited to, e.g., immune or dendritic cell-based vaccines (Seton-Rogers, S. (2012) Nature Reviews Cancer 12:230-231; Palucka, K. et al. (2012) Nature Reviews Cancer 12:265-277); effector memory CD8+ T cells (Bird, L. (2012) Nature Reviews Immunology 12:227); engineered tumor cells to activate Toll like Receptors (TLRs) and NOD-like Receptors (NLRB) (Leavy, O. (2012) Nature Reviews Immunology 12:227); antagonists of immune inhibitory signals or checkpoints (Pardoll, D. M. (2012) Nature Reviews Cancer 12:252-264). In one embodiment, the therapy is a cell-based immunotherapy wherein immune cells are expanded ex vivo and injected into the subject.

In yet other embodiments, the cancer therapy includes PDT used in combination with the AHCM, other cancer therapies, and/or the microenvironment modulator, described herein. In certain embodiments, PDT includes administration of a photosensitizing agent (e.g., a porhyrin, a porpyrin precursor, a chorlin, or a phthalocyanine) followed by irradiation at a wavelength corresponding to an absorbance band of the photosensitizing agent. In the presence of oxygen, a series of events lead to one or more of: cell death (e.g., tumor cell death), damage to the microvasculature, or induction of a local inflammatory reaction). PDT is reviewed in, e.g., Agostinis, P. et al. (2011) CA Cancer J. Clin. 61:250-281.

In other embodiments, the cancer therapy includes an inhibitor of a cancer stem cell (also referred to herein as a “cancer initiating cell”), used in combination with the AHCM, other cancer therapies and/or the microenvironment modulator, described herein. Without wishing to be bound by theory, hypoxia and cancer drugs (including anti-angiogenic drugs) and radiation therapy are believed to increase the number of cancer stem cells. AHCM, alone or in combination with, e.g., an inhibitor of a cancer stem cell, can be used to reduce the production of these stem cells. Exemplary inhibitors of cancer stem cells that can be used in combination include, but are not limited to, hedgehog (e.g., SMO) antagonists; and Wnt pathway antagonists (e.g., antibody, OMP-18R5). In one embodiment, the AHCM and/or the microenvironment modulator, alone or in combination with one or more cancer therapies described herein, are administered for cancer prevention (e.g., alone or in combination with cancer-prevention agents), during periods of active disorder, or during a period of remission or less active disorder. The AHCM and/or the microenvironment modulator, alone or in combination with one or more cancer therapies described herein, can be administered for cancer prevention, before treatment or prevention, concurrently with treatment or prevention, post-treatment or prevention, or during remission of the disorder. In one embodiment, the cancer therapy is administered simultaneously, sequentially, or a combination of both, with the AHCM and/or the microenvironment modulator.

In one embodiment, the ARCM and/or the microenvironment modulator is administered alone or in combination with cancer-prevention agents, e.g., to treat or prevent cancer in high risk subjects (e.g., a subject with pre-neoplasia or a genetic pre-disposition for cancer (e.g., a subject having a BRCA1 mutation); or a breast cancer patient treated with tamoxifen).

In some embodiments, the AHCM and/or the microenvironment modulator, alone or in combination with the cancer therapy, is a first line treatment for the cancer, e.g., it is used in a subject who has not been previously administered another drug intended to treat the cancer.

In other embodiments, the AHCM and/or the microenvironment modulator, alone or in combination with the cancer therapy, is a second line treatment for the cancer, e.g., it is used in a subject who has been previously administered another drug intended to treat the cancer.

In other embodiments, the AHCM and/or the microenvironment modulator, alone or in combination with the cancer therapy, is a third, fourth, or greater than fourth, line treatment for the cancer, e.g., it is used in a subject who has been previously administered two, three, or more than three, other drugs intended to treat the cancer.

In other embodiments, the AHCM and/or the microenvironment modulator is administered as adjunct therapy, e.g., a treatment in addition to a primary therapy.

In one embodiment, the AHCM and/or the microenvironment modulator is administered as adjuvant therapy.

In other embodiments, the AHCM and/or the microenvironment modulator is administered as neoadjuvant therapy.

In some embodiments, the AHCM and/or the microenvironment modulator is administered to a subject prior to, or following surgical excision/removal of the cancer.

In some embodiments, the AHCM and/or the microenvironment modulator is administered to a subject before, during, and/or after radiation treatment of the cancer.

In some embodiments, the AHCM and/or the microenvironment modulator is administered to a subject, e.g., a cancer patient who will undergo, is undergoing or has undergone cancer therapy (e.g., treatment with a chemotherapeutic agent, radiation therapy and/or surgery).

In other embodiments, the AHCM and/or the microenvironment modulator is administered prior to the cancer therapy. In other embodiments, the AHCM and/or the microenvironment modulator is administered concurrently with the cancer therapy. In yet other embodiments, the AHCM and/or the microenvironment modulator is administered prior to the cancer therapy and concurrently with the cancer therapy. In instances of concurrent administration, the AHCM and/or the microenvironment modulator can continue to be administered after the cancer therapy has ceased.

In other embodiments, the AHCM and/or the microenvironment modulator is administered sequentially with the cancer therapy. For example, the AHCM and/or the microenvironment modulator can be administered before initiating treatment with, or after ceasing treatment with, the cancer therapy. In one embodiment, the administration of the AHCM and/or the microenvironment modulator overlaps with the cancer therapy, and continues after the cancer therapy has ceased. In one embodiment, the ARCM and/or the microenvironment modulator is administered concurrently, sequentially, or as a combination of concurrent administration followed by monotherapy with either the cancer therapy, the AHCM, and/or the microenvironment modulator.

In one embodiment, the method includes administering the AHCM and/or the microenvironment modulator as a first therapeutic agent, followed by administration of a cancer therapy (e.g., treatment with a second therapeutic agent, radiation therapy and/or surgery). In another embodiment, the method includes administering a cancer therapy first (e.g., treatment with a first therapeutic agent, radiation therapy and/or surgery), followed by administering the AHCM and/or the microenvironment modulator as a second therapeutic agent. In yet other embodiments, the method includes administering the AHCM and/or the microenvironment modulator in combination with a second, third or more additional therapeutic agents (e.g., anti-cancer agents as described herein).

The AHCM and/or the microenvironment modulator and/or the anticancer agent described herein can be administered to the subject systemically (e.g., orally, parenterally, subcutaneously, intravenously, rectally, intramuscularly, intraperitoneally, intranasally, transdermally, or by inhalation or intracavitary installation). Typically, the AHCMs are administered orally. In certain embodiments, the AHCM and/or the microenvironment modulator and/or the anticancer agent are administered locally or intratumorally (e.g., via an oncolytic virus).

In some embodiments, the AHCM is administered as a pharmaceutical composition comprising one or more AHCMs, and a pharmaceutically acceptable excipient.

In an embodiment, the AHCM is administered, or is present in the composition, e.g., the pharmaceutical composition (e.g., the same nanoparticle composition).

In other embodiments, the AHCM, the microenvironment modulator and/or the cancer therapy are administered as separate compositions, e.g., pharmaceutical compositions (e.g., nanoparticle compositions). In other embodiments, the AHCM, the microenvironment modulator, and the cancer therapy are administered separately, but via the same route (e.g., orally or intravenously). In some embodiments, the AHCM, the microenvironment modulator, and the cancer therapy are administered by different routes (e.g., AHCM is administered orally; the microenvironment modulator is administered subcutaneoulsy; and a cancer therapeutic is administered intravenously). In still other instances, the AHCM, the microenvironment modulator, and the cancer therapy are administered in the same composition, e.g., pharmaceutical composition.

### Evaluating or Monitoring the Subject

The methods of the invention can further include the step of evaluating, or monitoring the subject, e.g., for one or more of: tumor size; the level or signaling of one or more of transforming growth factor beta 1 (TGFβ1), connective tissue growth factor (CTGF), thrombospondin-1 (TSP-1), or an angiotensin receptor (e.g., angiotension-II type-1 receptor; AT_{1A}, receptor (AT_{1A}R)); tumor collagen I levels; fibrotic content, interstitial pressure; a plasma, serum or tissue biomarker, e.g., collagen I, collagen III, collagen IV, TGFβ1, CTGF, TSP-1; levels of one or more cancer markers; the rate of appearance of new lesions, metabolism, hypoxia evolution; the appearance of new disease-related symptoms; the size of tissue mass, e.g., a decreased or stabilization; quality of life, e.g., amount of disease associated pain; histological analysis, lobular pattern, and/or the presence or absence of mitotic cells; tumor aggressivity, vascularization of primary tumor, metastatic spread; tumor size and location can be visualized using multimodal imaging techniques; or any other parameter related to clinical outcome. The subject can be evaluated or monitored in one or more of the following periods: prior to beginning of treatment; during the treatment; or after one or more elements of the treatment have been administered. Monitoring can be used to evaluate the need for further treatment with the same AHCM, alone or in combination with, the same microenvironment modulator and/or the same anti-cancer agent, or for additional treatment with additional agents. Generally, a decrease in one or more of the parameters described above is indicative of the improved condition of the subject.

In one embodiment, the method includes evaluating (e.g., detecting) the level of an angiotensin receptor (e.g., angiotension-II type-1 receptor; AT1A receptor (AT1AR) in the subject, e.g., in a tumor from the subject. Detection of the angiotensin receptor in the tumor from the subject indicates that the subject is likely to respond to the AHCM.

The methods of the invention can further include the step of analyzing a nucleic acid or protein from the subject, e.g., analyzing the genotype of the subject. The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the AHCM and/or anti-cancer agent, to thereby determine appropriate dosage(s) and treatment regimen(s) of the AHCM (e.g., amount per treatment or frequency of treatments) for prophylactic or therapeutic treatment of the subject.

### Dosage Forms

In another aspect, the invention features a pharmaceutically acceptable composition comprising, in a single dosage form, an ARCM and an anti-cancer agent, e.g., a small molecule or a protein, e.g., an antibody. In another embodiment, one or both of the ARCM and the anti-cancer agent are provided in a nanoparticle. The ARCM and anti-cancer agent can be in separate or the same entity. For example, if provided as separate entities the ARCM can be provided as a first nanoparticle and the anti-cancer agent provided as a second nanoparticle (e.g., where the second nanoparticle has a structural property (e.g., size or composition) or a functional property (e.g., release kinetics or a pharmacodynamic property) that differs from the first nanoparticle). Alternatively, an AHCM and an anti-cancer agent can be provided on the same entity, e.g., in the same nanoparticle.

In another aspect, the invention features a pharmaceutically acceptable composition (e.g., nanoparticle) comprising an AHCM, e.g., an AHCM described herein. In one embodiment, the AHCM is in a dosage described herein, e.g., a standard of care dosage form, a sub-anti-hypertensive dosage form, or a greater than a standard of care dosage form.

In one embodiment, the AHCM is formulated in a dosage form that is according to the standard of care anti-hypertensive or anti-heart failure dosage form, e.g., a standard of care dosage form as described herein.

In certain embodiments, the AHCM is formulated in a dosage form that is less than the standard of care anti-hypertensive or anti-heart failure dosage form (e.g., a dosage form that is less than 0.01, 0.02, 0.03, 0.04, 0.05, 0.06, 0.07, 0.08, 0.09, 0.1, 0.15, 0.16, 0.2, 0.3, 0.4, 0.5, 0.6, 0.7-fold, that of the standard of care dosage form, e.g., a standard of care dosage from as described herein).

In other embodiments, the AHCM is formulated in a dosage form that is greater than the standard of care anti-hypertensive or anti-heart failure dosage form (e.g., a dosage form that is greater than 1.1, 1.5, 1.7, 2, 3, 4, 5, 10-fold or higher, that of the standard of care dosage form, e.g., a standard of care dosage from as described herein).

In another aspect, the invention features a pharmaceutically acceptable composition comprising an anti-cancer agent, e.g., an anti-cancer agent described herein, as a nanoparticle, e.g., a nanoparticle configured for a method described herein.

In another aspect, the invention features a therapeutic kit that includes the AHCM, alone or in combination with a therapy, e.g., an anti-cancer agent, described herein, and optionally, instructions for use, e.g., for the treatment of cancer. In an embodiment, the kit comprises one or more dosage for or pharmaceutical preparation or nanoparticle described herein

### Delivery Methods

In another aspect, the invention features a method optimizing access to a target tissue, e.g., a cancer, or optimizing delivery to a target tissue, e.g., a cancer, of an agent, e.g., a systemically administered agent, e.g., a diagnostic or imaging agent. The method comprises:

administering an anti-hypertensive and/or a collagen modifying agent (“AHCM”) to the subject; and

optionally, administering an agent, e.g., a diagnostic or imaging agent to said subject.

In an embodiment, the method includes one or more of the following:

a) the AHCM is an anti-hypertensive agent and is administered at a standard of care dose, a sub-anti-hypertensive dose, or a greater than a standard of care—anti-dose;

b) the agent, e.g., diagnostic or imaging agent, has a hydrodynamic diameter of greater than 1, 5, or 20 nm, e.g., is nanoparticle;

c) the agent is an imaging agent, e.g., radiologic agent, an NMRA agent, a contrast agent; or

d) the subject is treated with a dosing regimen described herein, e.g., AHCM administration is initiated prior to administration of the agent, e.g., for at least one, two, three, or five days, or one, two, three, four, five or more weeks prior to administration of the agent.

In an embodiment, the AHCM is administered in an amount sufficient to alter (e.g., enhance) the distribution or efficacy of the agent. In one embodiment, the AHCM is administered in an amount sufficient to alter (e.g., enhance) the distribution or efficacy of the agent, but in an amount insufficient to inhibit or prevent tumor growth or progression by itself.

In an embodiment, the AHCM is administered at a dose that causes one or more of the following: a decrease in the level or production of collagen, a decrease in tumor fibrosis, an increase in interstitial tumor transport, improvement of tumor perfusion, or enhanced penetration or diffusion, of the cancer therapeutic in a tumor or tumor vasculature, in the subject.

In an embodiment, the subject is further treated with a cancer therapy, e.g., as therapy as described herein.

In an embodiment, the subject is a human, or a non-human animal, e.g., a mouse, a rat, a non-human primate, horse, or cow.

In another aspect, the invention features a diagnostic kit that includes the AHCM, alone or in combination with the agent, e.g., a diagnostic or imaging agent, described herein, and optionally, instructions for use, e.g., for the diagnosis of cancer.

### Screening Assays

In another aspect, the invention features a method, or assay for, identifying an AHCM. The method, or assay, includes providing a cancer or a cancer-associated cell (e.g., a culture of a carcinoma associated fibroblast cell); contacting said cancer or a cancer-associated cell with a candidate agent; detecting a change in the cancer cell in the presence, or absence, of the candidate agent. In one embodiment, the detected change includes one or more of an increase or decrease of TGFβ1 level, connective tissue growth factor (CTGF) level, or collagen (e.g., collagen 1) level. In one embodiment, the candidate agent is chosen from one or more of: an antagonist of renin angiotensin aldosterone system (“RAAS antagonist”), an angiotensin converting enzyme (ACE) inhibitor, an angiotensin II receptor blocker (AT₁ blocker), a thrombospondin 1 (TSP-1) inhibitor, a transforming growth factor beta 1 (TGF-β1) inhibitor, and a connective tissue growth factor (CTGF) inhibitor. A suitable candidate agent reduces one or more of TGFβ1 level (e.g., total and/or activated TGFβ1), connective tissue growth factor (CTGF) level, or collagen level.

The method, or assay, can further include the step of comparing the treated methods or assays to a reference value, e.g., a value obtained in the absence of the candidate agent, or by addition of a control agent, e.g., a positive agent (e.g., losartan), or a negative agent (e.g., saline control), and comparing the difference between the treated and control methods.

The method, or assay, can be performed in vitro, in vivo, or a combination of both. In one embodiment, the method, or assay, includes: evaluating the candidate agent in vitro, e.g., using a culture of carcinoma associated cells. In such embodiments, the candidate agent is added to the culture medium; and the condition medium is analyzed for an increase or decrease of TGFβ1 level, connective tissue growth factor (CTGF) level, or collagen level.

In another embodiment, the candidate agent is administered to a subject, e.g., an animal model, e.g., an animal tumor model. In such embodiments, the candidate agent is administered to the subject under suitable conditions; and the subject is analyzed for an increase or decrease of TGFβ1 level, connective tissue growth factor (CTGF) level, or collagen level. In one embodiment, the levels of these parameters are analyzed as described in the appended Examples.

In yet other embodiments, candidate agents evaluated using the in vitro assays are tested in vivo.

In another aspect, the invention features a composition for use, or the use, of a AHCM agent, alone or in combination with an anti-cancer agent described herein for the treatment of a cancer or tumor described herein.

Headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 is a panel a bar graphs depicting the effects of losartan (10 μmol/L) in total and active TGFβ levels, and collagen I synthesis by carcinoma associated fibroblasts (CAFs) in vitro.

FIGS. 2A-2B shows the effects of Losartan on collagen production in tumors.

FIG. 2A shows a panel of photographs showing a dose-dependent reduction in collagen levels assessed by SHG imaging in losartan-treated HSTS26T tumors, as compared to the control, over a period of two weeks, (10, 20 and 60 mg/kg/day). Scale bar=200 μm.

FIG. 2B shows a dose response curve of the effect of losartan doses of 10, 20 and 60 mg/kg/day in decreasing the SHG levels by 20, 33 and 67%, respectively, at the end of 15 days, indicating a dose-dependent reduction in collagen levels in Losartan-treated tumors. There was a statistically significant difference (*) between the control group and the two higher doses (20 and 60 mg/kg/day). There was also a statistically significant difference (†) between the 20 and 60 mg/kg/day groups.

FIG. 3 is a bar graph showing a dose response of losartan vs. collagen content in HSTS26T tumors. Losartan treatment at 20 and 60 mg/kg/day led to 42% and 63% reduction in collagen I staining respectively. The staining in each treatment group was compared to a control group that received saline.

FIG. 4 is a bar graph showing the effect of losartan in decreasing the mean arterial blood pressure (MABP) in mice in a dose-dependent manner. Although 20 mg/kg/day decreased MABP by 10 mm Hg (*p<0.04), the MABP remained within the normal range for SCID mice (70 mmHg-95 mmHg)(13). Conversely, when animals were treated with 60 mg/kg/day, the 35 mm Hg (**p<0.04) drop in MABP was lower than the normal range MABP in SCID mice.

FIGS. 5A-5D shows the effects of Losartan in collagen levels in tumors.

FIG. 5A shows the results of Collagen-I and nuclei immunostaining in tumor sections in L3.6 μl and MMTV control and losartan (20 mg/kg/day) treated tumors. Scale bar=100 μm. Losartan treatment (e.g., at 20 mg/kg/day) significantly reduced the collagen levels in the treated tumors.

FIG. 5B is a bar graph summarizing the effects after two weeks losartan treatment at 20 mg/kg/day; losartan treatment significantly reduced the collagen I immunostaining in L3.6 μl (p<0.03) and FVB MMTV PyVT by 50% (p<0.05) and 47% (p<0.05), respectively.

FIG. 5C is a panel of photographs showing collagen-I and nuclei immunostaining in tumor sections in HSTS26T and Mu89 control and losartan (20 mg/kg/day) treated tumors. Note that there is no detectable reduction in collagen I immunostaining at 200 μm from the edge of HSTS26T tumors. This phenomenon is less obvious in treated Mu89 tumors where there is some persistent staining both at the edge and in central tumor areas. Scale bar=100 μm.

FIG. 5D is a bar graph summarizing the effects of Losartan in significantly reducing the collagen-I immunostaining in HSTS26T and Mu89 by 44% (p<0.02) and 20% (p<0.05), respectively.

FIG. 6 is a panel of bar graphs showing the effects of losartan on TSP-1, active and total TGF-β1, and collagen I in HSTS26T tumors. Treated animals received losartan (15 mg/kg/day) in drinking water. Tumors were excised after two weeks of treatment, homogenized and analyzed for total and activated TGF-β1 levels by ELISA. Note a 3.5 fold reduction in TSP-1, a 4 fold reduction in active TGF-β1 and a two fold reduction in collagen 1 after losartan treatment (p<0.05).

FIG. 7A shows the effects of losartan in decreasing tumor TSP-1 immunostaining in both MU89 and HSTS26T tumors. In HSTS26T tumors, the changes in TSP-1 after losartan treatment correspond with changes in collagen I immunostaining; TSP-1 levels decrease in the tumor center but remain high within a 200 μm from the edge of the tumor. The TSP-1 margin was larger (500 μm from the edge) in MU89 tumors. Scale bar=100 μm.

FIG. 7B is a bar graph summarizing the effects of losartan treatment in significantly reducing the TSP-1 immununostaining in HSTS26T and MU89 tumors by 73% (p<0.04) and 24% (p<0.03), respectively.

FIGS. 8A-8C shows the effects of: Losartan in increasing the delivery of nanoparticles and nanotherapeutics.

FIG. 8A shows two photographs (control and losartan) and a bar graph summarizing the distribution of intratumorally (i.t.) injected 100 nm diameter nanoparticles in HSTS26T tumors. Losartan significantly increased (p<0.001) the distribution of i.t.-injected nanoparticles in both tumor types (1.5 fold in HSTS26T and 4 fold in Mu89). An analysis of the distribution pattern shows control tumors with fewer intratumoral nanoparticles and a majority of nanoparticles that backtracked out of the needle track and accumulated at the tumor surface. In contrast, treated tumors have a significant number of intratumoral nanoparticles. Scale bar=100 μm.

FIG. 8B shows two photographs (control and losartan) and a bar graph summarizing the distribution of viral infection 24 hrs after the intratumoral injection of HSV expressing the green fluorescent protein. HSV infection in control tumors is limited to the cells in close proximity to the injection site whereas losartan treated tumors have a more extensive spread of HSV infection within the tumors. Scale bar=1 mm. Losartan significantly increased (p<0.05) the virus spread in HSTS26T and Mu89 tumors.

FIG. 8C shows two photographs (control and losartan) and a bar graph summarizing the distribution of intravenously (i.v.) injected 100 nm diameter nanoparticles in L3.6 μl tumors. The nanoparticles are localized around perfused vessels. There is a two-fold increase (p<0.05) in nanoparticle content in losartan-treated tumors compared to control tumors. Scale bar=100 μm.

FIG. 9 is a bar graph showing the changes in diffusion coefficient in HSTS26T tumors after losartan treatment. The diffusion coefficient of IgG was measured in HSTS26T tumors implanted in the dorsal window chamber of SCID mice. Treated animals received (40 mg/kg/day) losartan by i.p. injection while control animals received saline. The results show a significant increase (p<0.04) in diffusion coefficient as measured by multiphoton fluorescence recovery after photobleaching (FRAP).

FIG. 10 is a representative distribution profile depicting fractions of injected nanospheres present as a function of the distance from a tumor vessel (penetration depth). The nanosphere penetration depth was analyzed in frozen sections from tumors resected 24 hrs after the intravenous nanosphere injection. The mean characteristic penetration length increased from 18±5 μm (mean±SE) in control to 37±6 pin in losartan-treated tumors. Ten areas per tumor were analyzed in 6 control and 6 treated tumors.

FIG. 11A-11D shows the effects of Losartan in significantly delaying the growth of tumors treated with DOXIL® or HSV.

FIGS. 11A-11B shows linear graphs of the results from mice bearing HSTS26T (A) and Mu89 (B) tumors treated for 2 weeks with either losartan or saline prior to the i.t. injection of HSV. Losartan alone did not affect the growth of Mu89 or HSTS26T tumors. The growth delay was significantly longer in HSTS26T tumors treated with losartan and HSV compared to tumors treated with HSV alone. The i.t. injection of HSV did not delay the growth of Mu89 tumors, but the combined losartan and HSV treatment significantly retarded the growth of Mu89 tumors.

FIG. 11C shows the effect in tumor volume in mice that received losartan treatment prior to i.v. DOXIL® infusion (losartan and DOXIL®) have smaller tumors than those that received DOXIL® alone (DOXIL® alone) in L3.6 μl tumors. Note that there is no difference in tumor size between saline and losartan-treated mice.

FIG. 11D is an image showing a clear difference in size between control tumors (left column) and losartan treated tumors (right column) at 1 week after DOXIL® infusion. Scale bar=lcm. The losartan treated tumors (right column) were smaller than the control tumors (left column) at 1 week after DOXIL® infusion.

FIGS. 12A-12B shows the relationship between the collagen structure and the virus infection and necrosis.

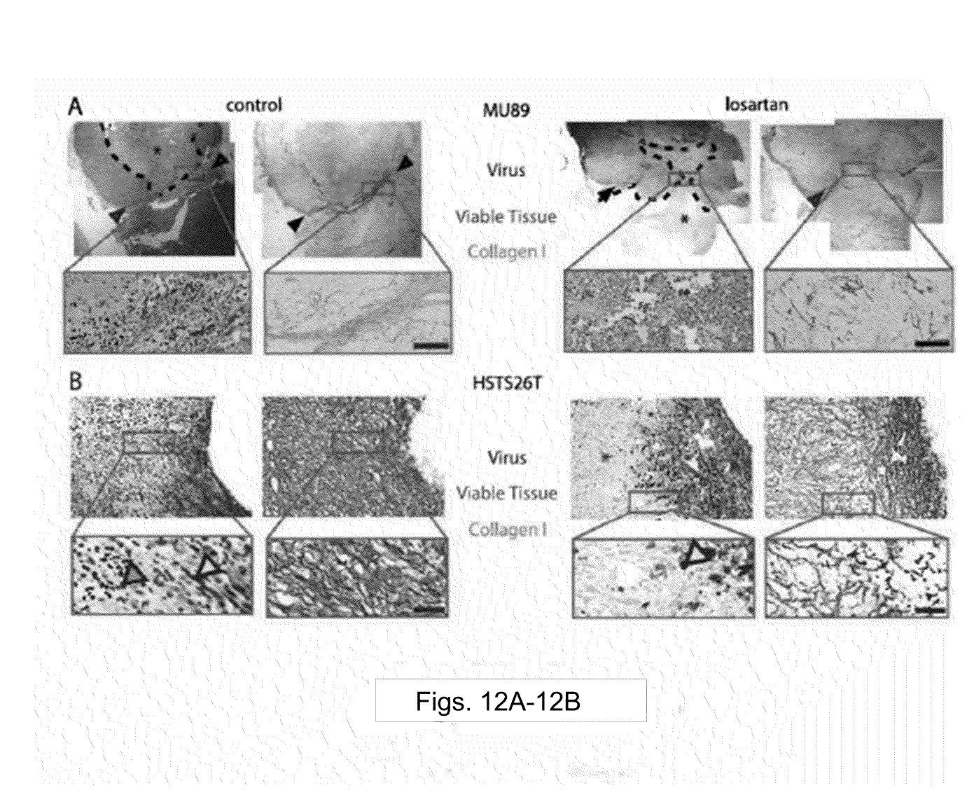

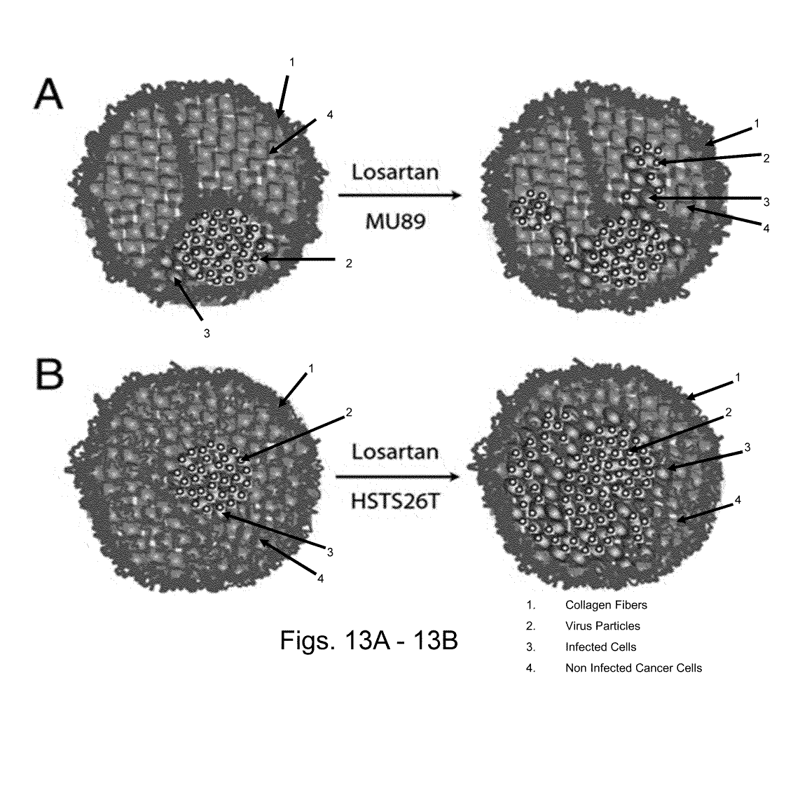

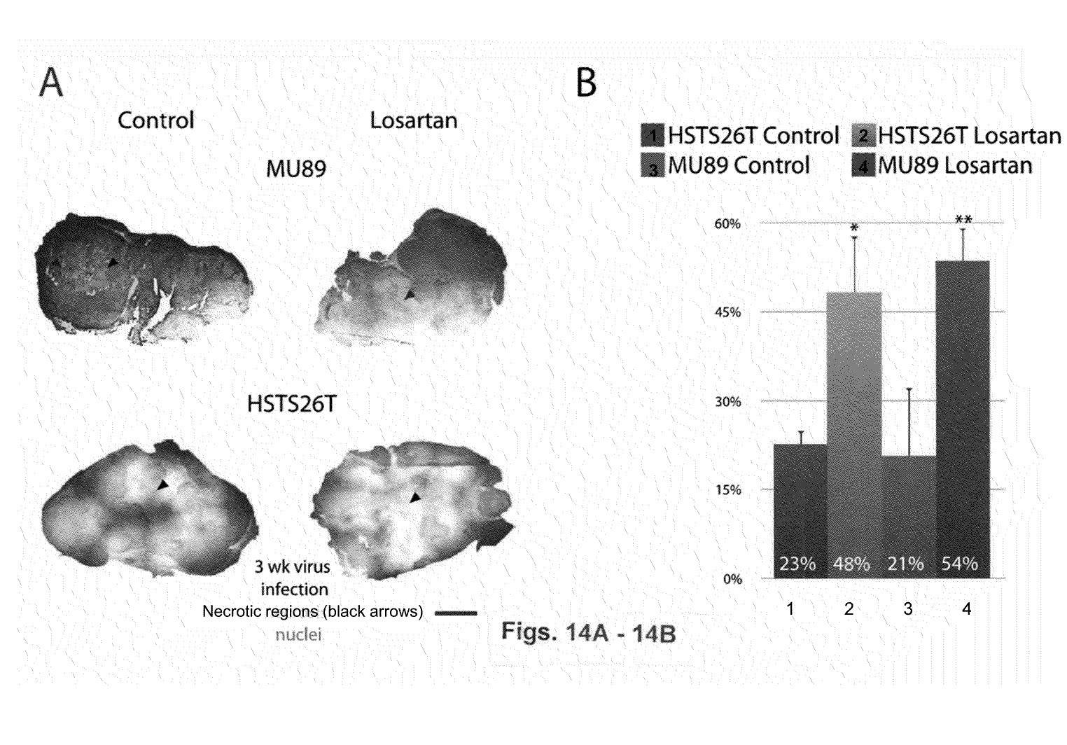

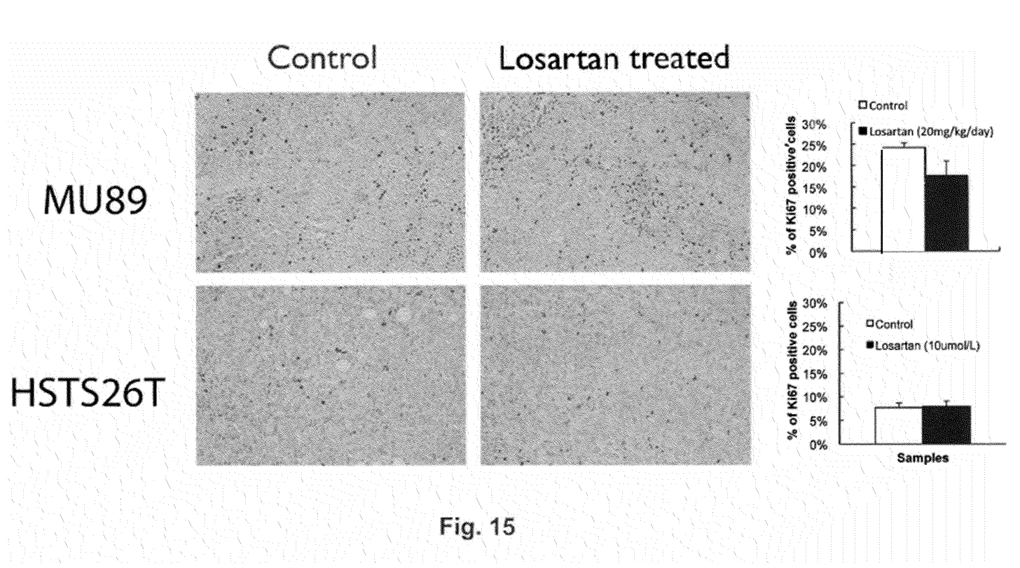

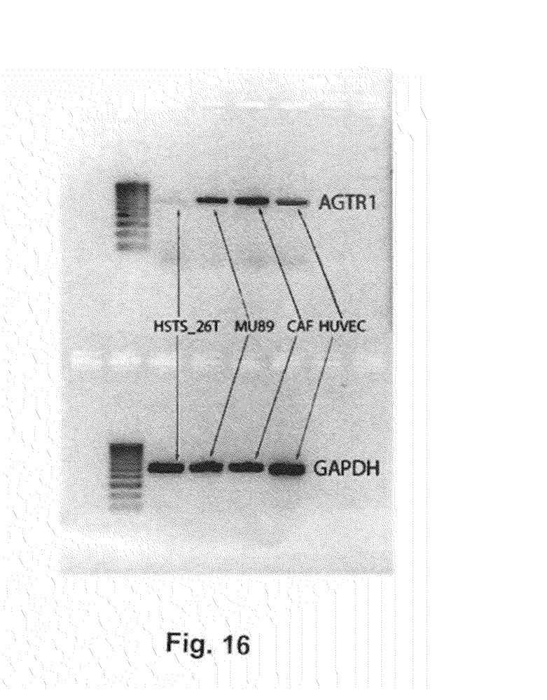

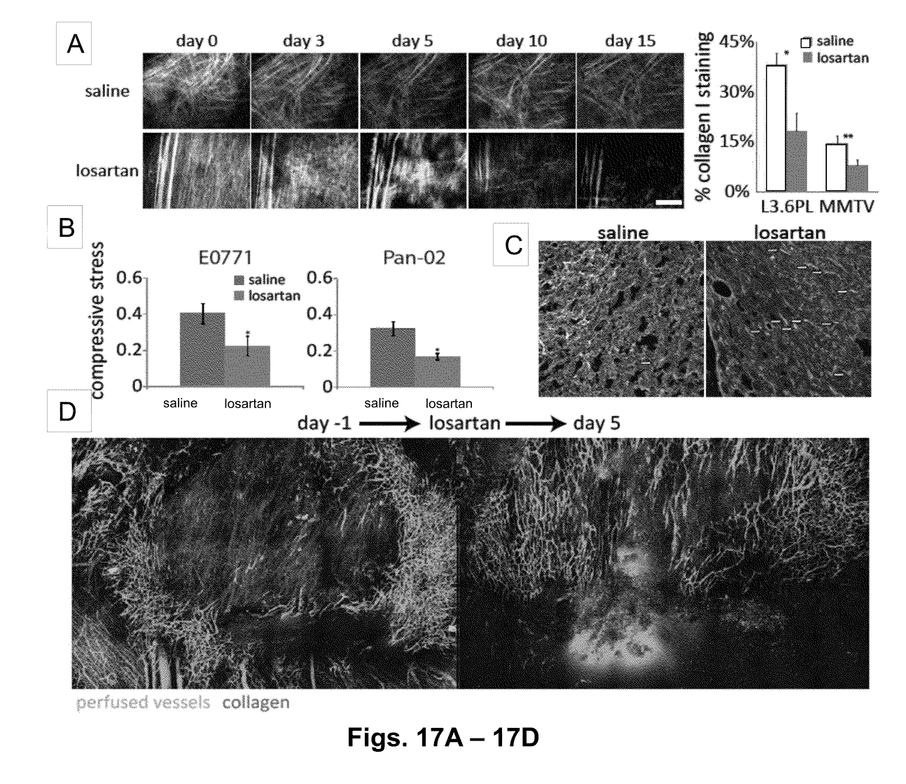

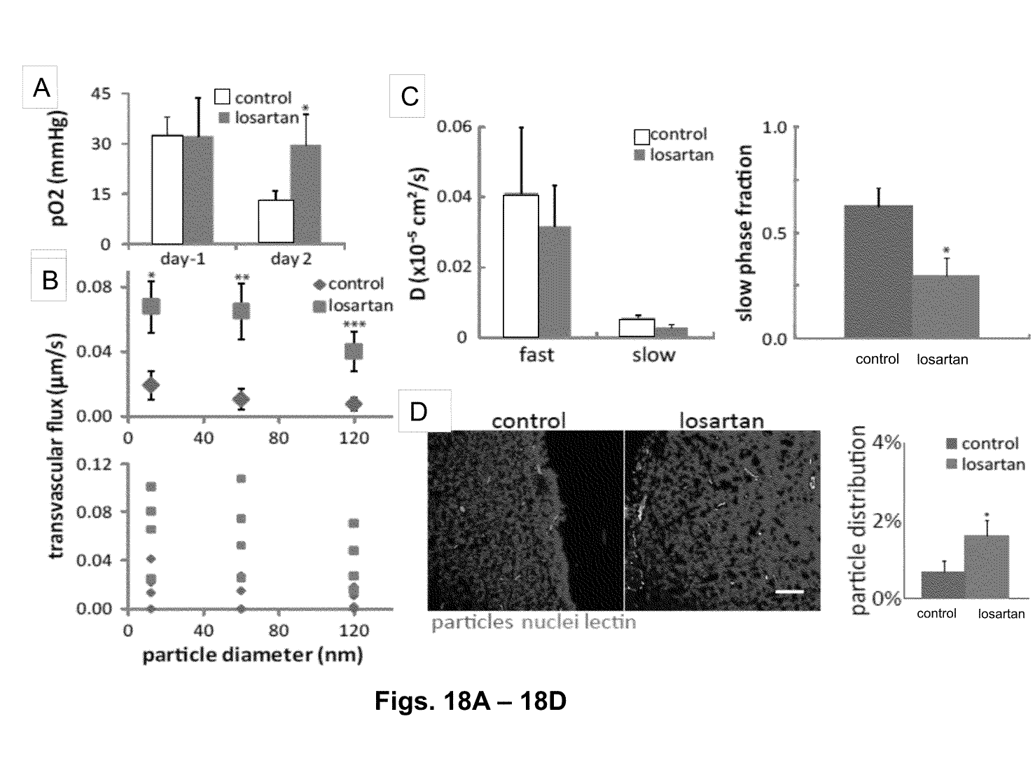

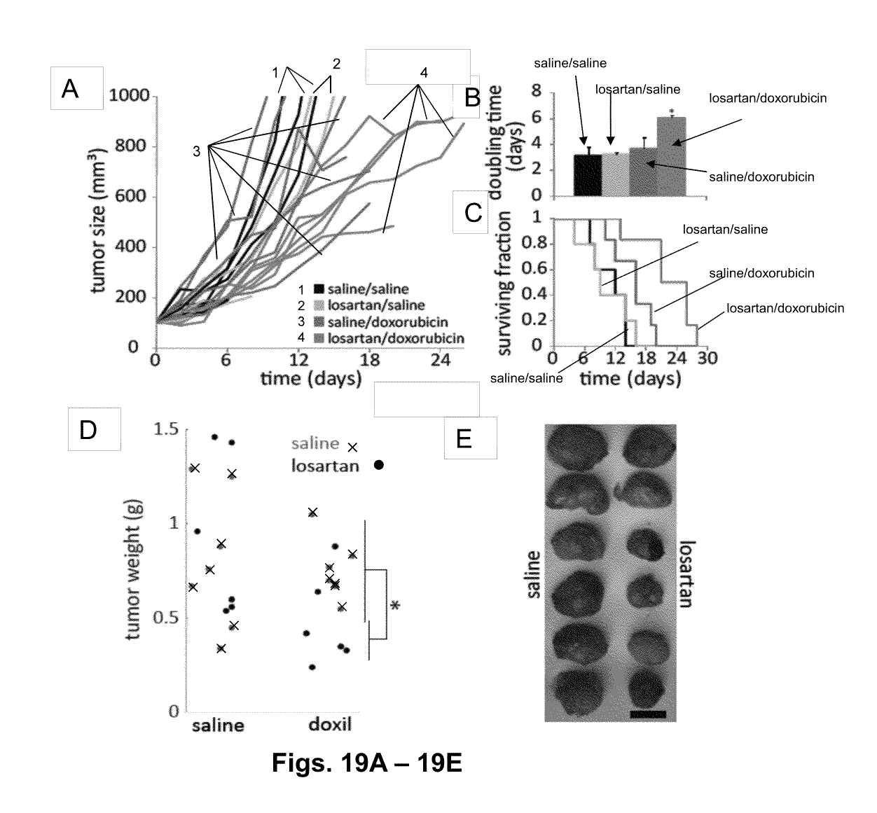

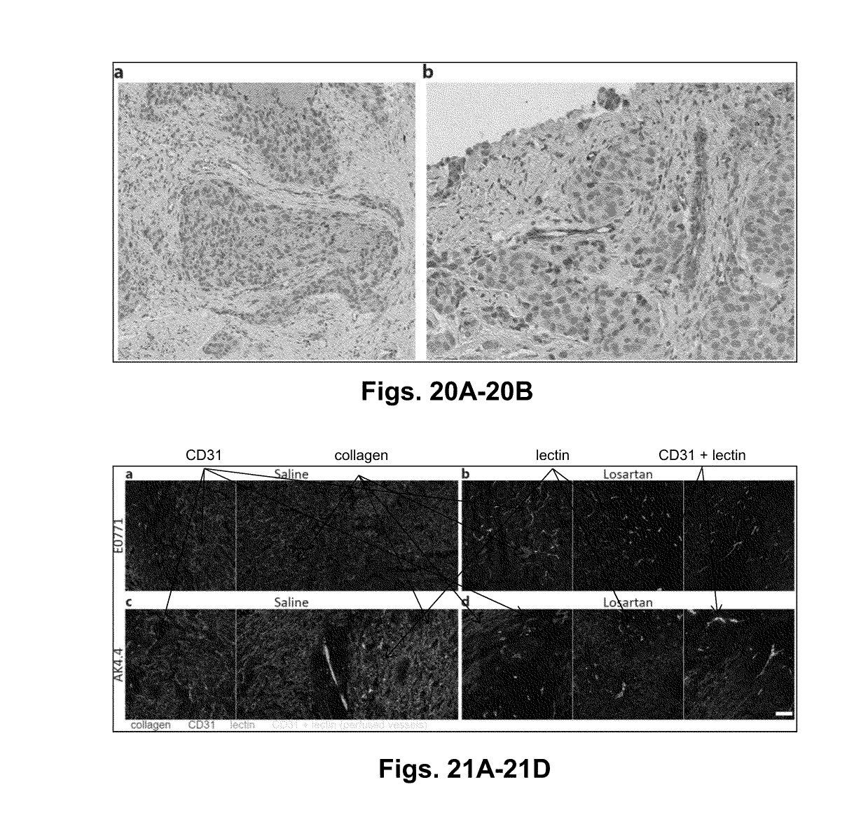

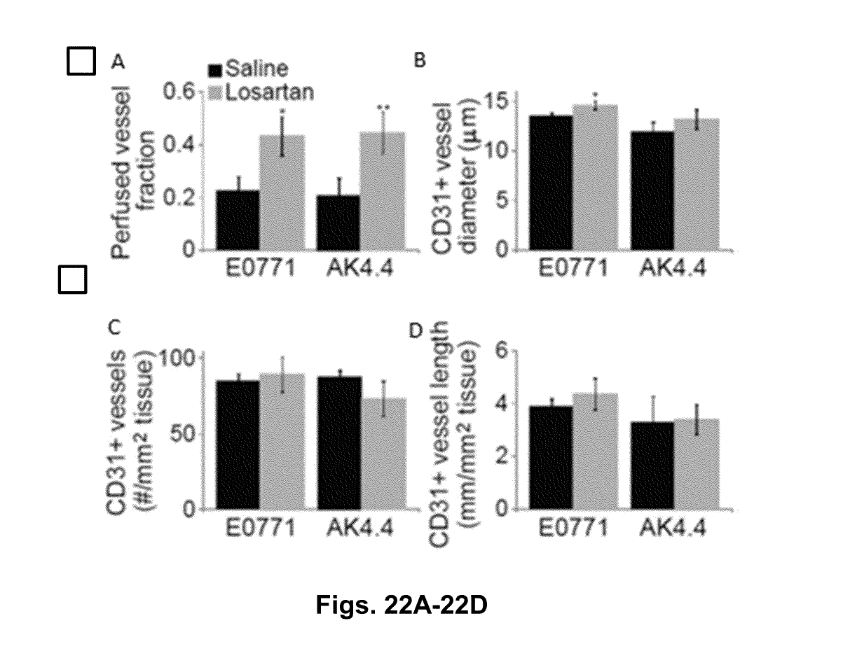

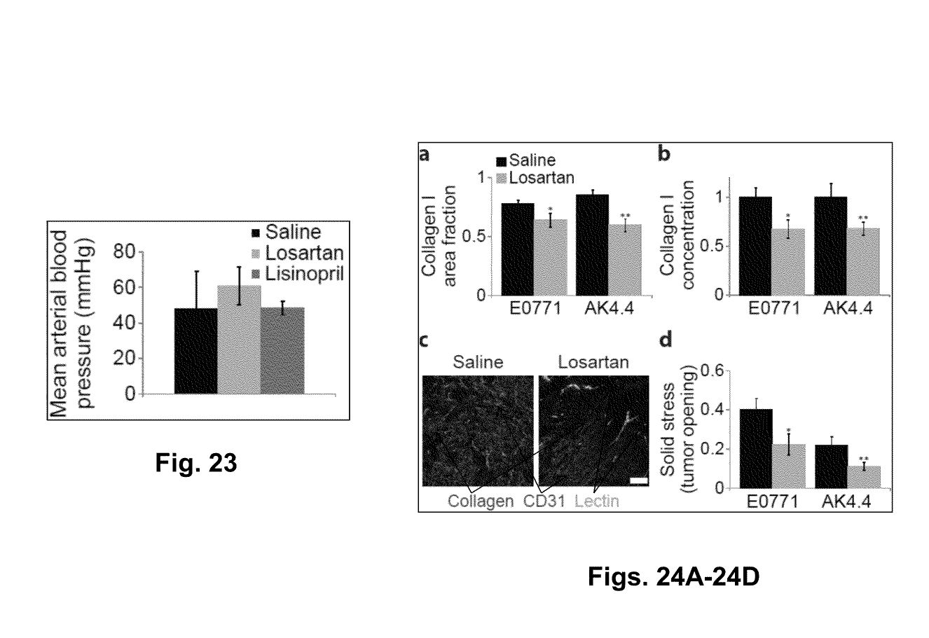

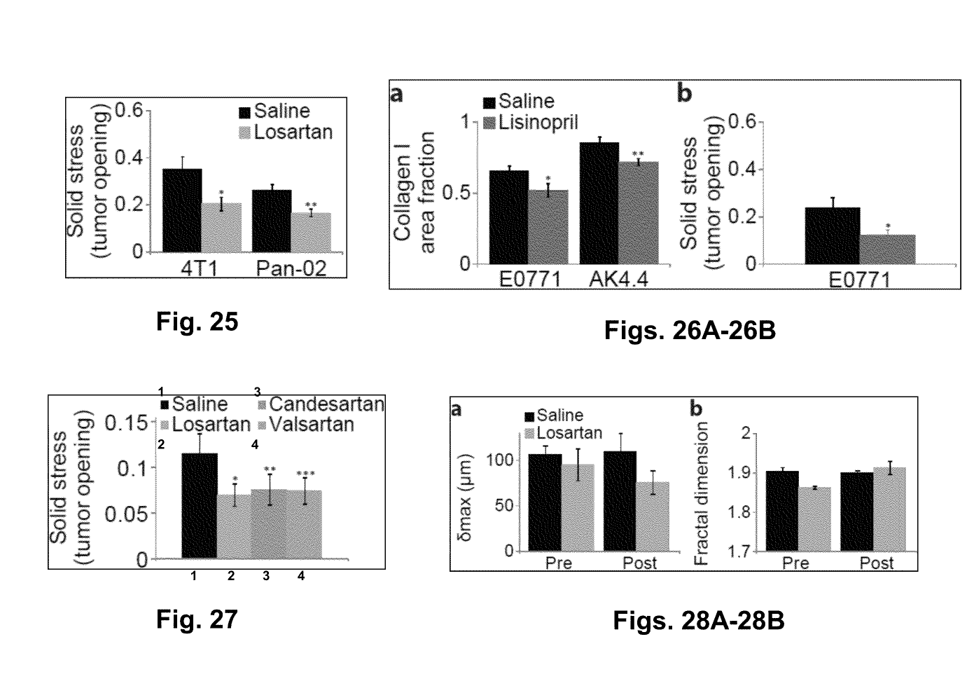

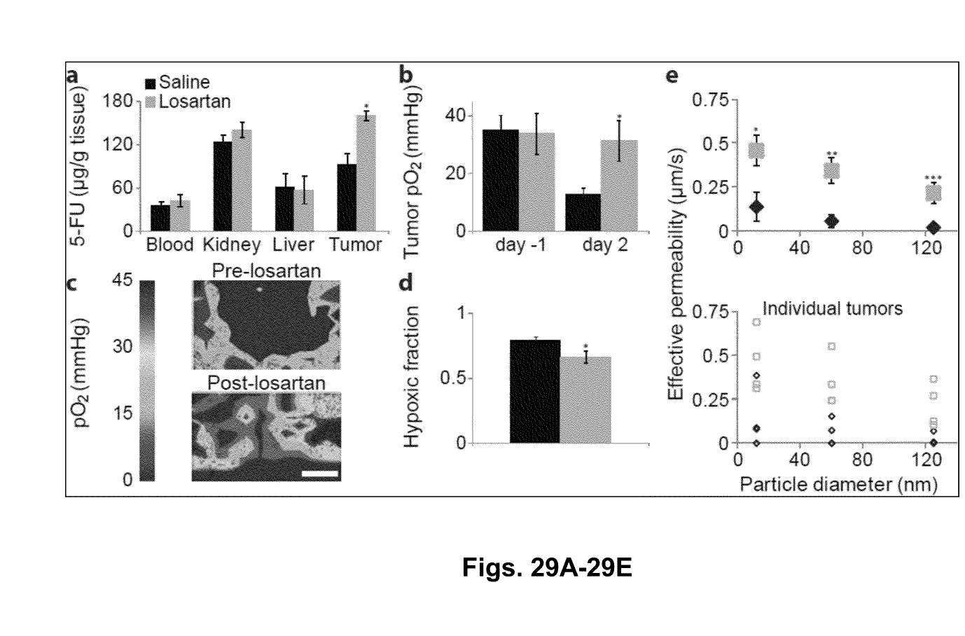

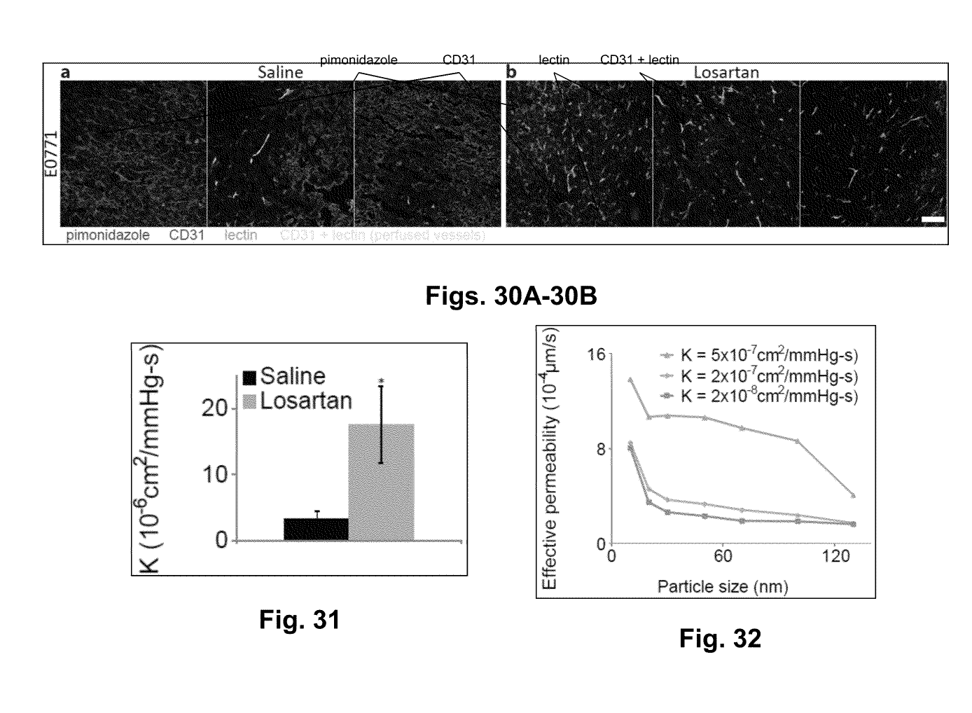

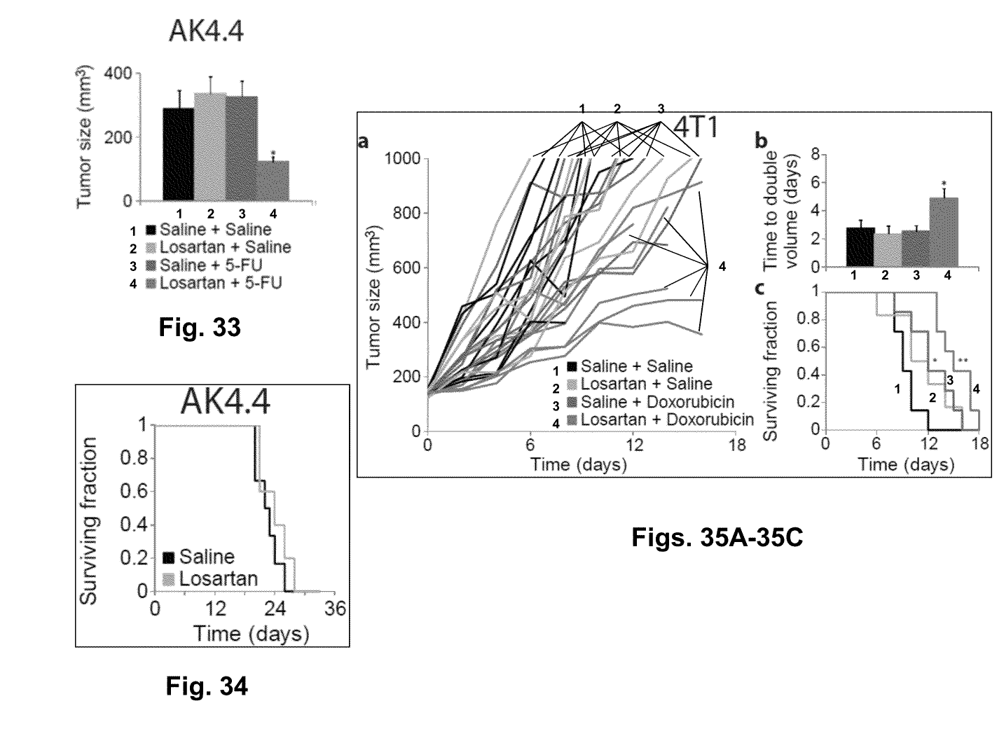

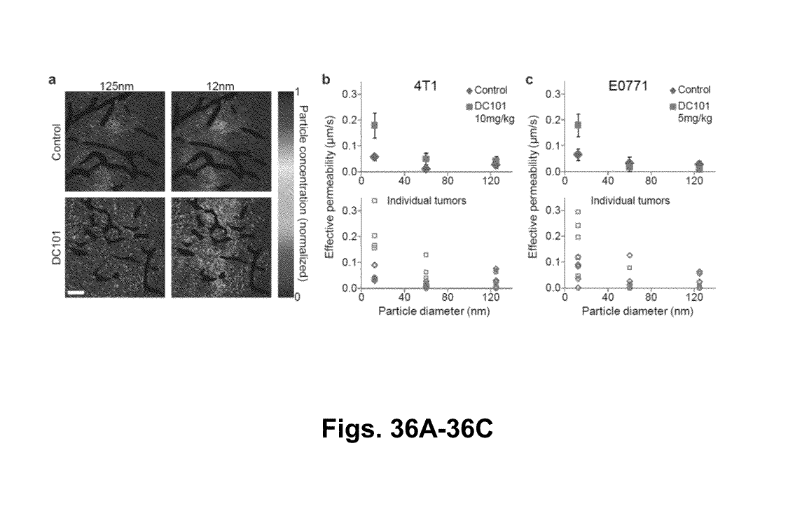

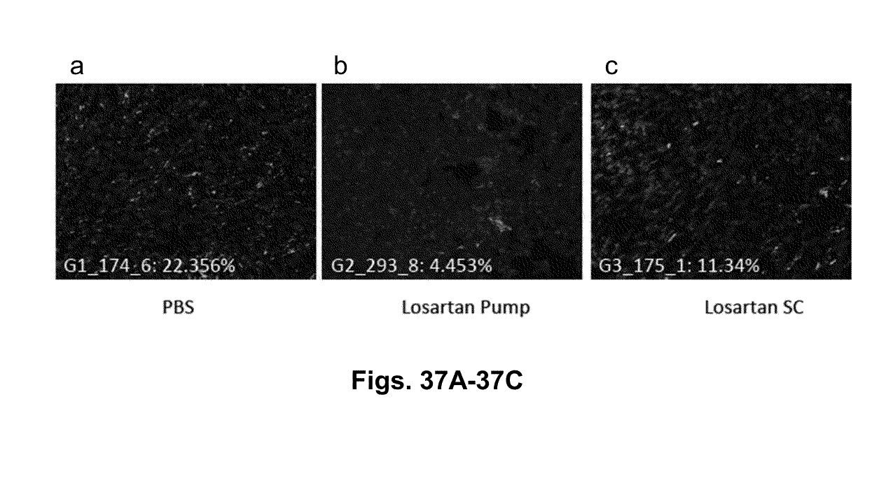

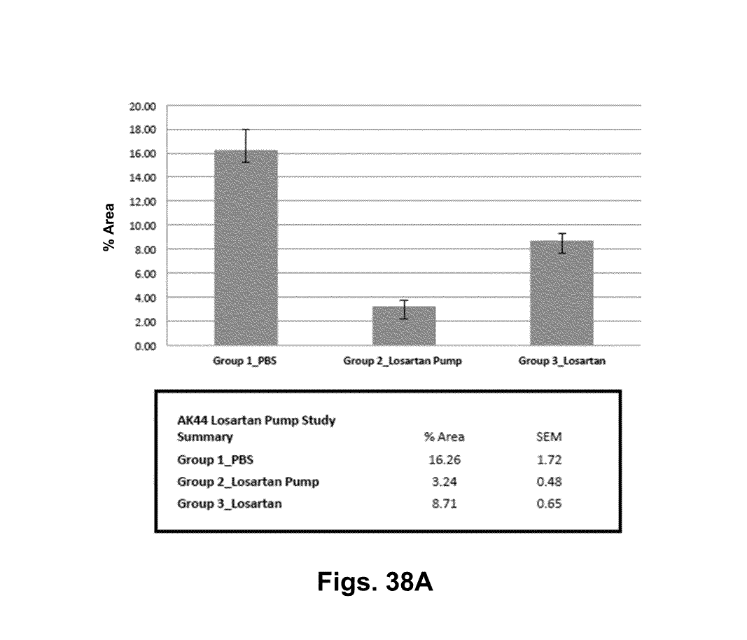

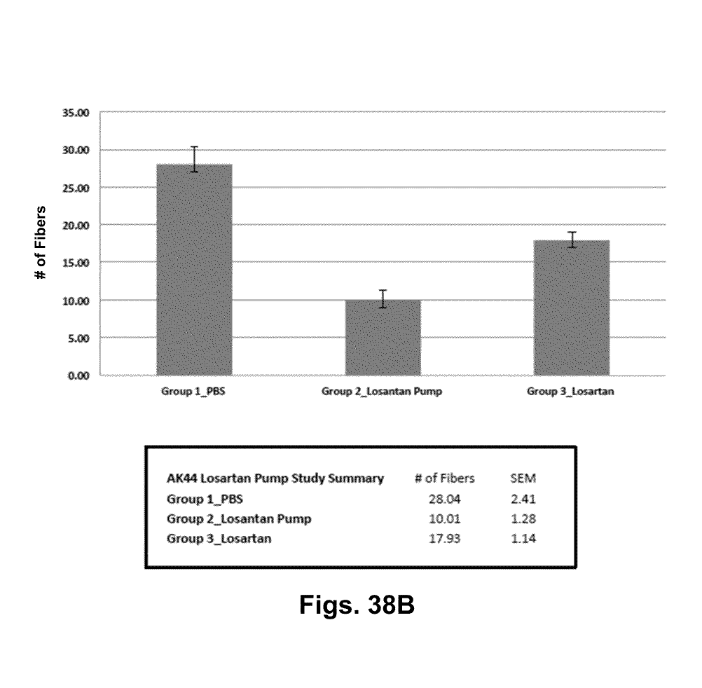

In FIG. 12A, Mu89 tumors collagen bundles are seen around the tumor margin. Occasionally, these bundles project into the tumor (black arrows) and divide the tumor into separate compartments. These compartments seem to confine movement of HSV; evident from the containment of the necrotic region within the region bounded by collagen bundles. When these tumors were treated with losartan the collagen bundles at the margins of the tumor remained intact but the projections became less organized (insert). This presumably allowed virus propagation and necrosis to extend across the boundaries. Scale bar=100 μm.

In FIG. 12B, HSTS26T, the dense mesh-like collagen network confined virus infection to the immediate area surrounding the injection point. With losartan treatment, there was a reduction in the density of the network that presumably allowed virus particles to infect a larger area and thus more tumor cells. Arrows indicate viable and virus infected cells, respectively. Scale bar=10 μm.

FIGS. 13A-13B shows a schematic of virus distribution and infection in Mu89 (A) and HSTS26T (B) tumors. The schematics show how the different collagen network structures affect virus propagation and distribution. The collagen fibers (1) restrict the movement of virus particles (round spheres, 2) and the infection (3) of non-infected (4) cancer cells.

In FIG. 13A, Mu89 tumors, collagen bundles divide the tumor into isolated regions that cannot be traversed by virus particles. Losartan treatment destabilizes the collagen bundles and allows virus particles to move from one region to another.

In FIG. 13B, HSTS26T tumors, the collagen structure is a mesh-like sieve. Virus particles can still propagate through the sieve but do not extend very far from the injection site. Losartan treatment significantly destabilizes the mesh structure in the internal regions of the tumor and allows the virus to propagate and infect a larger area.

FIG. 14A shows virus infection (HSV immunostaining) and necrosis 21 days after HSV injection in HSTS26T and MU89. Hematoxylin staining of intact tumor areas, necrosis, and HSV immunostaining is shown. Necrotic regions are indicated by black arrows. Even though there was no detectable difference in necrotic area between HSTS26T and MU89, necrosis is confined to specific regions in MU89 while there is necrotic tissue (bounded by HSV immunostaining) throughout HSTS26T tumors. Scale bar=2 mm.

FIG. 14B is a bar graph showing that there is a two-fold increase (p<0.05) in necrosis in tumors (both HSTS26T and MU89) that received losartan prior to HSV injection.

FIG. 15 shows the in vivo proliferation rates for HSTS26T and MU89 after losartan treatment. Tumors were resected and stained for Ki67 to assess proliferation rates. There was no statistically significant difference in positive Ki67 staining after losartan treatment in HSTS26T and MU89 tumors. There was however a significant difference in proliferation between the two tumor types, the number of Ki67 positive cells was 3 fold higher in HSTS26T tumors.

FIG. 16 shows the results of PCR analysis of AGTR1 expression in CAF, MU89 and HSTS26T cells. MU89 cells and CAF express AGTR1 while HSTS26T cells do not. HUVECs were used as a positive control. GAPDH levels revealed that all three samples had roughly the same amount of cDNA.

FIGS. 17A-17D shows the effects of angiotensin blockade with AT₁ blockers or ACE inhibitors in normalizing the tumor microenvironment. Studies with an ARB, losartan, are shown. Angiotensin blockade (A) diminishes interstitial matrix density in mammary (MMTV) and pancreatic (L3.6PL) tumors in mice, (B) reducing compressive stress in mammary (E0771) and pancreatic (Pan-02) tumors. (C) This increases the fraction of perfused vessels (arrows) in tumors (E0771 shown), resulting in (D) a normalized vascular network (E0771 shown) that is more efficient and effective at drug and oxygen delivery.

FIGS. 18A-18D shows the effects of angiotensin blockade with AT₁ blockers or ACE inhibitors in improving drug transport and distribution in tumors. Studies with an ARB, losartan, are shown here. Through tumor normalization, angiotensin blockade (A) improves tumor oxygenation (E0771 shown) through enhanced perfusion while (B) making blood vessels deliver drugs more rapidly. Reorganization of the interstitial matrix also (C) improves penetration of nanoparticles in desmoplastic tumors (L3.6PL shown) (D).

FIGS. 19A-19E shows the effect of angiotensin blockade with AT₁ blockers or ACE inhibitors in improving the effectiveness of cancer therapy. Studies with an ARB, losartan, are shown. Angiotensin blockade, given in combination with chemotherapy (A) improves the effectiveness of the low MW chemotherapeutic doxorubicin in breast cancer models, (B) slowing tumor growth and (C) increasing animal survival (E0771 shown). Similarly, angiotensin blockade (D, E) improves the effectiveness of the nanoparticle DOXIL® in pancreatic tumors (L3.6PL shown), e.g., by decreasing the tumor weight (D) and/or tumor size or volume (E).

FIGS. 20A-20B shows the compression of tumor blood vessels in human breast cancer. Biopsies of tumors from breast ductal adenocarcinoma patients were stained for CD31-positive vessels. Unbridled cell proliferation in the confined microenvironment of these tumor and stromal cells results in vessel compression in the stroma (A) and within tumor nodes (B). All vessels appear to be compressed to some degree, with many completely collapsed.

FIGS. 21A-21D are histology images of mouse tumors showing collagen I (blue), CD31-positive vessels (red), and lectin-positive vessels (green), with CD31-lectin co-staining (yellow) denoting perfused vessels. Representative stainings were shown by arrows. In FIGS. 21A-21D, angiotensin inhibitors improve perfusion of tumor blood vessels. Control E0771 breast tumors (A) are dense with collagen I and vessels, yet only a small fraction of these vessels are perfused. Control AK4.4 pancreatic tumors (C have higher collagen I levels and a lower vessel density, with vessels that are also poorly perfused. Losartan improves perfusion in E0771 (B) and AK4.4 (D) by decreasing collagen I levels, without anti-angiogenic effects. Scale bar, 100 μm.

FIGS. 22A-22D are bar graphs showing that angiotensin inhibitors improve vascular perfusion.

In FIG. 22A, the perfused vessel fractions, measured by histology with lectin and CD31 co-staining after angiotensin inhibition using losartan, are shown. Following lectin injection and animal sacrifice, perfusion was quantified as the fraction of vessels that are both lectin- and CD31-positive out of all CD31-positive vessels. Losartan increases the fraction of vessels that are perfused in orthotopic E0771 breast (P=0.038, Student's t-test) and AK4.4 pancreatic (P=0.039, Student's t-test) tumors.

In FIG. 22B, the CD31-positive vessel diameter, measured using histology after angiotensin inhibition with losartan, is shown. Losartan also increases vessel diameter in E0771 tumors (P=0.047, Student's t-test), indicating decompression as the mechanism.

In FIGS. 22C-22D, the CD31-positive vessel density, measured using histology following angiotensin inhibition using losartan, is shown. Losartan does not affect vessel density, as quantified by the vessel number density (C) and the total vessel length (D), indicating no anti-angiogenic effect at this 40 mg/kg dose Animal number n=7-9 for all groups.

FIG. 23 is a bar graph showing that angiotensin inhibitors do not decrease blood pressure at certain doses. Mean arterial blood pressure measured by coronary artery cannulation in mice bearing AK4.4 pancreatic tumors. Losartan and lisinopril treatment at a 40 mg/kg dose does not lower blood pressure in these tumor-bearing mice. Overall, blood pressure in these diseased mice is lower than in healthy FVB mice (˜90 mmHg).

FIGS. 24A-24D show the effect of angiotensin inhibitors on decompressing vessels by reducing solid stress.

In FIGS. 24A-24B, the tumor matrix levels following angiotensin inhibition with losartan are shown. Losartan decreases matrix production, quantified by collagen I area fraction (A), in orthotopic E0771 breast (P=0.043, Student's t-test) and AK4.4 pancreatic (P=0.018, Student's t-test) tumors. Losartan also reduces the concentration of collagen I (B) in E0771 (P=0.048, Student's t-test) and AK4.4 (P=0.050, Student's t-test) tumors, confirming a reduction in matrix levels.

FIG. 24C are representative histology images of lectin, CD31, and collagen I staining. Representative staining were shown by arrows. A high local collagen I concentration appears to colocalize with collapsed vessels, suggesting that elevated matrix levels in the microenvironment of a tumor vessel directly lead to compression. Scale bar, 100 μm.

In FIG. 24D, the solid stress levels in tumors after angiotensin inhibition using losartan are shown. Solid stress was assessed using an ex vivo technique involving the measurement of the extent of tumor tissue relaxation (tumor opening relative to tumor diameter) following a stress-releasing incision, with larger openings indicating higher stress. Through its anti-matrix effects, losartan reduces solid stress in E0771 (P=0.049, Student's t-test) and AK4.4 (P=0.043, Student's t-test) Animal numbers n=5-7 (E0771 collagen), n=4-6 (AK4.4 collagen), n=5 (E0771 stress), n=8-9 (AK4.4 stress).

FIG. 25 is a bar graph showing that angiotensin inhibitors decrease stress in multiple tumor models. Solid stress levels in tumors after angiotensin inhibition using losartan are shown. Through its anti-matrix effects, losartan reduces solid stress in 4T1 breast tumors (P=0.036, Student's t-test) and Pan-02 pancreatic tumors (P=0.0092, Student's t-test). Animal numbers n=10-11 (4T1 stress), n=4-8 (Pan-02 stress).

FIGS. 26A-26B are bar graphs showing that the ACE-I lisinopril decreases matrix levels and solid stress.

In FIG. 26A, the tumor matrix levels following angiotensin inhibition with lisinopril are shown. Lisinopril decreases matrix production, quantified by collagen I area fraction in orthotopic E0771 breast (P=0.048, Student's t-test) and AK4.4 pancreatic (P=0.031, Student's t-test) tumors.

In FIG. 26B, the solid stress levels in tumors after angiotensin inhibition using lisinopril are shown. Through its anti-matrix effects, lisinopril reduces solid stress in E0771 (P=0.050, Student's t-test) Animal numbers n=6 (E0771 collagen), n=4 (AK4.4 collagen), n=7 (E0771 stress).

FIG. 27 is a bar graph showing that a panel of ARBs reduces solid stress. Solid stress levels in tumors after angiotensin inhibition using the ARBs losartan, candesartan, and valsartan are shown. Doses were chosen based on their relative doses in patients for hypertension indications (40 mg/kg losartan, 3.2 mg/kg candesartan, 32 mg/kg valsartan). Losartan (P=0.0069, Student's t-test) candesartan (P=0.0091, Student's t-test), and valsartan (P=0.0091, Student's t-test) all reduce solid stress to a similar degree. Animal numbers n=6-7. Statistical tests were corrected for multiple comparisons using the Holm-Bonferroni method.

FIGS. 28A-28B are bar graphs showing that angiotensin inhibitors result in a normalized network of perfused vessels. Mathematical analysis of perfused vessel network efficiency for delivery was conducted. Perfused vessel networks of E0771 tumors were imaged in three dimensions using multiphoton microscopy pre- and post-treatment (days 2-5). Analysis of the distance from each point in the tumor to the nearest perfused vessel (A) indicates that losartan decreases the maximum distance drugs and oxygen must travel to reach tumor cells. Fractal analysis of vessel network structure (B) shows that losartan increases the fractal dimension from a typical 1.89 in tumors toward the usual 2.0 of normal capillary beds. Together, these data suggest that increasing perfusion with angiotensin inhibitors leads to a more normal vascular network structure.

FIGS. 29A-29E are graphs showing that angiotensin inhibitors increase drug and oxygen delivery.

FIG. 29A shows small-molecule drug delivery to tumors and various organs after angiotensin inhibition with losartan. Losartan increases the accumulation of the small-molecule chemotherapeutic 5-FU in AK4.4 pancreatic tumors by 74% (P=0.0063, Student's t-test) while not affecting accumulation in the normal organs.

FIGS. 29B-29C show oxygen delivery to tumors measured by phosphorescence quenching microscopy during angiotensin inhibition using losartan. Losartan maintains the level of oxygenation (B) in the tissue, versus control tumors that become progressively more hypoxic with time (P=0.030, Student's t-test). Losartan increases oxygenation in some tumors (C), whereas all control tumors decrease in oxygen levels. Losartan also appears to result in a more homogenous distribution of well-oxygenated tumor tissue. Scale bar, 100 μm.

FIG. 29D shows the hypoxic fraction in tumors measured by pimonidazole injection and staining following angiotensin inhibition with losartan. Losartan decreases the hypoxic fraction in E0771 tumors (P=0.027, Student's t-test) due to the increase in oxygen delivery.

FIG. 29E shows the penetration rates for nanoparticles after angiotensin inhibition with losartan. Penetration rates are quantified as effective permeability, which is the transvascular mass flux per unit vascular surface area and transvascular concentration difference. Closed symbols (top) denote averages by mouse, while open symbols (bottom) are individual tumors. Losartan enhances nanoparticle delivery in a largely size-independent manner, improving the penetration of 12 nm (P=0.039, Student's t-test), 60 nm (P=0.013, Student's t-test), and 125 nm (P=0.022, Student's t-test) nanoparticles. Animal numbers n=4 (drug delivery, nanoparticle penetration), n=6 (oxygen delivery), n=7-8 (hypoxia).

FIGS. 30A-30B are histology images of mouse tumors showing that angiotensin inhibitors reduce hypoxia. Pimonidazole hypoxia staining (blue), CD31-positive vessels (red), and lectin-positive vessels (green) are shown, with CD31-lectin co-staining (yellow) denoting perfused vessels. Representative stainings were shown by arrows. Control E0771 breast tumors (A) show pronounced hypoxia away from the few vessels that are perfused. Losartan improves perfusion, reducing hypoxia (B). Scale bar, 100 μm.

FIG. 31 is a bar graph showing that angiotensin inhibitors increase fluid flow in tumors. Interstitial hydraulic conductivity was measured by the flow rate of media through freshly excised tumor tissue. Losartan increases fluid flow through E0771 breast tumors, demonstrating a large increase in the interstitial hydraulic conductivity (P=0.035, Student's t-test). Animal number n=6.

FIG. 32 is a line graph showing that increasing fluid flow in tumors can improve nanoparticle penetration. Predictions of physiologically-based mathematical model of how modulating interstitial hydraulic conductivity can improve nanoparticle penetration are shown. Increasing interstitial hydraulic conductivity results in more rapid penetration rates (effective permeability) for all sizes of nanoparticles by allowing for more rapid fluid flow driven by the difference in the microvascular pressure and the interstitial fluid pressure at the tumor margin.

FIG. 33 is a bar graph showing that angiotensin inhibitors synergistically enhance chemotherapy effectiveness. Volumes of orthotopic AK4.4 pancreatic tumors on day 7 in response to treatment with losartan or saline control (40 mg/kg daily from day 0-7) in combination with either the small-molecule chemotherapeutic 5-FU or saline control (60 mg/kg on days 2 and 6) are shown. 5-FU and losartan monotherapy induce no significant growth delay versus the control treatment, whereas their combination greatly inhibited tumor growth (P=0.0085, Student's t-test) Animal number n=5-6 for all groups. Statistical tests were corrected for multiple comparisons using the Holm-Bonferroni method.

FIG. 34 is a survival curve showing that angiotensin inhibitors do not decrease survival Animal survival following tumor implantation, with initiation of treatment with losartan on day 11, is whon. Losartan monotherapy does not affect survival versus saline. Animal number n=5-6.

FIGS. 35A-35C are graphs showing that angiotensin inhibitors enchance chemotherapy in multiple models.

In FIG. 35A, the volumes of orthotopic 4T1 breast tumors in response to treatment with losartan or saline control (40 mg/kg daily from day 0 on) in combination with either the small-molecule chemotherapeutic doxorubicin or saline control (2 mg/kg every 3 days from day 1 on) are shown.

In FIG. 35B, the quantification of tumor growth rates, based on the time to reach double the initial volume, is shown. Doxorubicin and losartan monotherapy induce no significant growth delay versus the control treatment in these aggressive tumors. In contrast, their combination greatly limits tumor growth (P=0.024, Student's t-test).

In FIG. 35C, the animal survival following the initiation of treatment is shown. Doxorubicin monotherapy improves survival versus the control (P=0.045, log-rank test), while the combination of doxorubicin and losartan enhances this survival increase versus doxorubicin monotherapy (P=0.050, log-rank test) Animal number n=6-7 for all groups. Statistical tests were corrected for multiple comparisons using the Holm-Bonferroni method.

FIGS. 36A-36C show the effects of vascular normalization using anti-angiogenic therapy on nanoparticle delivery in tumors

In FIG. 36A, nanoparticle penetration versus particle size in orthotopic 4T1 mammary tumors in response to normalizing anti-angiogenic therapy with the VEGF receptor inhibitor, DC101. Nanoparticle concentrations are relative to initial intravascular levels, with vessels in black. Normalization improves 12 nm particle penetration, while not detactably affecting 125 nm penetration. Scale bar, 100 μm.

In FIGS. 36B-36C, penetration rates (effective permeability) for nanoparticles in orthotopic 4T1 and E0771 mammary tumors in mice treated with 10 mg/kg or 5 mg/kg DC101, respectively. Closed symbols (top) denote averages by mouse, while open symbols (bottom) are individual tumors. Normalization improves the penetration rate of 12 nm particles on day 2 by a factor of 3.1 in 4T1 (P=0.042, Student's t-test) and 2.7 in E0771 (P=0.049, Student's t-test), while not improving delivery for larger nanoparticles. Normalization also reduces the flux of large nanoparticles to zero in several individual tumors Animal number n=5 for all groups.

FIG. 37A-37C are representative images of immunohistochemiccal staining for collagen I in AK4.4 tumor samples from mice administered with vehicle (PBS) or losartan.

In FIG. 37A, mice were subcutaneously injected with PBS (Group 1 or G1). In FIG. 37B, mice were administered with losartan via subcutaneous pump (Group 2 or G2). In FIG. 37C, mice were subcutaneously injected with losartan in the absence of pump (Group 3 or G3). The percentages of collagen I positive areas are 22.356%, 4.453%, and 11.34% for Groups 1-3, respectively.

FIG. 38A is a bar graph showing the average percentages of collagen I positive areas in tumor samples from mice subcutaneously injected with PBS (Group 1), administered with losartan via subcutaneous pump (Group 2), and subcutaneously injected with losartan in the absence of pump. The average percentages of collagen I positive areas are 16.26±1.72%, 3.24±0.48%, and 8.71±0.65% for Groups 1-3, respectively.

FIG. 38B is a bar graph showing the average numbers of collagen I fibers in tumor samples from mice subcutaneously injected with PBS (Group 1), administered with losartan via subcutaneous pump (Group 2), and subcutaneously injected with losartan in the absence of pump. The average numbers of collagen I positive fibers are 28.04±2.41, 10.01±1.28, and 17.93±1.14 for Groups 1-3, respectively.

### DETAILED DESCRIPTION OF THE INVENTION

The invention is based, at least in part, on the discovery that anti-hypertensive and/or collagen-modifying (AHCM) agents (including angiotensin inhibitors, e.g., angiotensin receptor blockers (e.g., losartan) and angiotensin-converting enzyme inhibitors (ACE-I) improve the delivery and efficacy of cancer therapeutics.

The abnormal matrix of tumors limits the delivery of nano-therapeutics in many types of cancer, e.g., pancreatic, breast, lung, colorectal. The overgrowth of fibrous tissue impedes the movement of nanotherapeutics in tumors two mechanisms—viscoelastic and steric hindrances. Fibrous tissue is highly viscoelastic, meaning it is quite thick and stiff, and therefore slows the movement of these drugs to a small fraction of their typical speed. This tissue is basically an extremely dense mesh, with small pores that are about the same size as nanotherapeutics, thus it does not allow much space for these drugs and often halts their movements by confining them close to blood vessels (in case of intravenous injection) or near the site of injection (in case of intra-tumor injection). This barrier is found in all solid tumors, with possible exception of brain tumors, though it is most prominent in pancreatic, breast, lung, and colorectal cancers. Nanotherapeutics, owing to their large size relative to the pores that form the tumor microenvironment, are especially hindered by fibrous tissue.

In certain embodiments disclosed herein, administration of losartan prevents the production of matrix molecules like collagen, which are a component of the dense mesh of fibrous tissue. Without being bound by theory, losartan is believed to act on fibroblasts and tumor cells by inhibiting the TGF-beta and CTGF pathways, thus limiting their pro-fibrotic activity. It does so by blocking the activity of the angiotensin-II type-1 receptor (AT1), which is highly expressed on both fibroblasts and tumor cells in a variety of cancers. Thus losartan blocks activity downstream of AT1 in various signaling pathways, including the activation of TGF-beta and CTGF. Since these two pathways promote the production of collagen and other components of fibrotic tissue, blocking them will allow the fibrosis to subside. The result is tissue that is much more like the normal surrounding organ, and is therefore easier to penetrate.

Treatment with losartan is shown herein to significantly reduce collagen levels—a marker of fibrosis—in several types of tumors, including pancreatic, breast, skin, and soft tissue tumors. Furthermore, reduction in fibrosis leads to improved mobility of nanotherapeutics in tumors, allowing them to penetrate tumors more easily, and allows these drugs to distribute more widely throughout tumors, making them more effective at fighting tumor growth. Hence, losartan makes nanotherapeutics more effective against cancer.

In embodiments, it has been discovered that losartan normalizes the collagen, interstitial matrix of several solid tumors, thus facilitating the penetration of chemotherapeutics, such as large molecular weight (e.g., nano-) chemotherapeutics. For example, losartan reduced collagen I levels in carcinoma associated fibroblasts (CAFs) isolated from breast cancer biopsies, and caused a dose-dependent reduction in stromal collagen in desmoplastic models of human breast, pancreatic and skin tumors in mice. Losartan also improved the distribution, therapeutic efficacy and/or penetration of nanopartices (e.g., oncolytic herpes simplex viruses (HSV) and pegylated liposomal doxorubicin (DOXIL®)).

Low molecular weight therapeutics, which are much smaller than nanotherapeutics, are not as limited by the interstitial matrix barriers, but are similarly affected by other barriers such as abnormal and collapsed blood vessels.

In other embodiments, losartan is shown to facilitate decompression of blood vessels, thus improving tumor perfusion and delivery of low molecular weight chemotherapeutics, thus facilitating radiation and chemotherapeutic delivery through vascular normalization.

Thus, these agents improve delivery of molecules as small as oxygen—a radiation and chemo sensitizer—through vascular normalization (FIGS. 18A-18B), while also enhancing the penetration of larger agents through interstitial matrix normalization (FIG. 18C, 18D). Through this repair of the entire tumor microenvironment, these agents enhance the effectiveness of low molecular weight chemotherapeutics, as well as nanotherapeutics in breast and pancreatic cancer models—leading to reduced tumor growth and longer animal survival (FIGS. 19A-19E).

Further examples disclosed herein demonstrate a reduction in collagen levels and tumor solid stress using angiotensin inhibitors (other than losartan), including, for example, angiotensin receptor blockers (ARBs), such as candesartan and valsartan, as well as angiotensin converting enzyme inhibitors (ACE-I), such as lisinopril (see e.g., FIGS. 26-27).

Therefore, angiotensin inhibitors (e.g., angiotensin receptor blockers) and ACE inhibitors can enhance the delivery of a therapy, and thus have broad applicability for combination therapy with all classes of anti-cancer agents, including low molecular weight, small-molecule chemotherapeutics, biologics, nucleic acid agents and nanoparticle therapies.

The AHCM described herein (e.g., angiotensin inhibitors, such as angiotensin receptor blockers and ACE inhibitors) can be used in combination with a microenvironment modulator to enhance penetration and/or diffusion, of a cancer therapy in a tumor or tumor vasculature, in a subject. Such combination may cause one or more of: reduce solid stress (e.g., growth-induced solid stress in tumors); decrease tumor fibrosis; reduce interstitial hypertension or interstitial fluid pressure (IFP); increase interstitial tumor transport; increase tumor or vessel perfusion; increase vascular diameters and/or enlarge compressed or collapsed blood vessels; reduce or deplete one or more of: cancer cells, or stromal cells (e.g., tumor associated fibroblasts or immune cells); decrease the level or production of extracellular matrix components, such as fibers (e.g., collagen, procollagen), and/or polysaccharides (e.g., glycosaminoglycans such as hyaluronan or hyaluronic acid); decrease the level or production of collagen or procollagen; decreases the level or production of hyaluronic acid; increases tumor oxygenation; decreases tumor hypoxia; decreases tumor acidosis; enable immune cell infiltration; decreases immunosuppression; increases antitumor immunity; or decreases cancer stem cells, thereby enhancing the penetration and/or distribution of the cancer therapy.

Exemplary microenvironment modulators are disclosed herein, and include, but are not limited to, an anti-angiogenic therapy, for example, an inhibitor of vascular endothelial growth factor (VEGF) pathway; an agent that decreases the level or production of hyaluronic acid; an inhibitor of the hedgehog pathway; an agent that improves drug penetration in tumors (e.g., a disulfide-based cyclic RGD peptide peptide (iRGD) or an analogue thereof); a taxane therapy (e.g., taxane-induced apoptosis); an agent that decreases the level or production of collagen or procollagen; and/or a profibrotic pathway inhibitor as described herein.

Angiotensin blockers offer numerous advantages over other approaches, including anti-angiogenic therapies, anti-collagen agents and other matrix modifiers. For example, anti-angiogenic therapies normalize the vasculature alone and have been approved for only a limited number of indications. Meanwhile, ARBs and ACE-Is are FDA-approved as anti-hypertensives with manageable adverse effects. Morover, anti-angiogenics, which are FDA-approved adjuncts that enhance drug delivery to tumors, tend not to improve the delivery for larger particles as they can reduce the size of “pores” in vessel walls. More specifically, vascular normalization with anti-angiogenic therapies can typically enhance the delivery and effectiveness of small therapeutics, including small molecule chemotherapeutics, biologics and small nanoparticles (e.g., in the range of 1-12 nm), while not substantially affecting the delivery of larger therapeutics (e.g., about 50 nm to about 100 nm, e.g., about 60 nm or larger) (data shown in FIGS. 36A-36C). In addition, anti-collagen agents, such as relaxin, can improve transport through the tumor matrix, but not facilitate the delivery of low molecular weight agents (see e.g., U.S. Pat. No. 6,719,977). In contrast, AHCM (e.g., angiotensin blockers) can improve delivery for a broader size range and class of anti-tumor diagnostics and therapies, including low molecular weight, small-molecule chemotherapeutics, biologics, nucleic acid agents and nanoparticle therapies (as described in the Examples herein).

Matrix modifiers like bacterial collagenase, relaxin, and matrix metalloproteinase-1 and -8 have been used to modify the collagen or proteoglycan network in tumors and have improved the efficacy of intratumorally (i.t.) injected oncolytic viruses (Brown E, et al. (2003) Nat Med 9:796-800; McKee T D, et al. (2006) Cancer Res 66:2509-2513; Mok W, et al. (2007) Cancer Res 67:10664-10668; Ganesh S, et al. (2007) Cancer Res 67:4399-4407; and Kim J-H, et al. (2006) J Natl Cancer Inst 98:1482-1493). However, these agents may produce normal tissue toxicity (e.g., bacterial collagenase) or increase the risk of tumor or metastatic progression (e.g., relaxin, matrix metalloproteinases). For example, matrix-degrading enzymes, which can normalize the collagen matrix, are not selective for tumors and can increase invasion and metastasis. Other approaches for improving interstitial transport may also cause increased metastasis. For example, relaxin, a hormone produced during pregnancy that modulates collagen fiber structure to improve diffusion of nano-sized probes (Brown, E. et al. (2003) Nat. Med. 9(6):796-800; Perentes, J. Y. et al. (2009) Nat. Methods 6(2):143-5), may lead to increased metastasis, perhaps due to the mechanism of relaxin as a matrix-degrading therapy.

In contrast, ARBs and ACE-Is have no significant complications associated with matrix remodeling in normal tissues, leading to their safety as anti-hypertensives. In the cancer context, Applicants show that losartan monotherapy did not significantly increase metastasis in the cancer model tested, AK4.4; and losartan combination with 5-FU appeared to reduce the incidence and size of metastases (Table 2). Thus, angiotensin inhibitors, such as ARBs and ACE-Is, are likely to cause less metastasis than other anti-collagen agents, such as matrix-degrading enzymes and relaxin.

Another advantage of ARBs and ACE-Is, as small-molecule agents, is that they can also be delivered via nanovectors containing chemotherapeutics (e.g., liposomes, nano-particles) to enhance their localization to tumors to further limit toxicity.

Thus, methods and compositions for improving the delivery and/or efficacy of cancer therapeutics are disclosed. Methods and compositions for treating or preventing a cancer (e.g., a solid tumor such as a desmoplastic tumor) by administering to a subject an anti-hypertensive agent, as a single agent or combination with a microenvironment modulator and/or a cancer therapeutic agent (for example, a therapeutic agent ranging in size from a large nanotherapeutic to a low molecular weight chemotherapeutics and/or oxygen) are disclosed.

Certain terms are first defined.

“About” and “approximately” shall generally mean an acceptable degree of error for the quantity measured given the nature or precision of the measurements. Exemplary degrees of error are within 20 percent (%), typically, within 10%, and more typically, within 5%, 4%, 3%, 2% or 1% of a given value or range of values.

“Delivery,” as used herein in the context of delivery of an agent(s) to a tumor, refers to the placement of the agent(s) in sufficient proximity to one or more (or all) of: the tumor vasculature, the tumor interstitial matrix, or tumor cells or tumor-associated cells (e.g., fibroblasts), to have a desired effect. The agent(s) can be, e.g., a cancer therapy (e.g., a cancer therapeutic agent(s) as described herein), or a diagnostic or imaging agent(s). Unless noted otherwise, the term “agent” or “agent(s)” as used generically herein can include one, two or more agents.

In one embodiment, the cancer therapeutic agent includes, e.g., one or more of a small molecule, a protein or a nucleic acid drug, an oncolytic virus, a vaccine, an antibody or a fragment thereof, or a combination thereof. The cancer therapeutic agent can be “free” or packaged or formulated into a delivery vehicle, e.g., a particle, e.g., a nanoparticle (e.g., a lipid nanoparticle, a polymeric nanoparticle, or a viral particle). Delivery of a therapeutic agent is characterized by placement of the therapeutic agent in sufficient proximity to the cell to alter an activity of the cell, e.g., to kill the cell and/or reduce its ability to divide.

In other embodiments, the agent is a diagnostic or an imaging agent (e.g., one or more of a radiologic agent, an NMRA agent, a contrast agent, or the like). The diagnostic or imaging agent can be “free” or packaged or formulated into a delivery vehicle. Delivery of a diagnostic or imaging agent is characterized by placement of the agent in sufficient proximity to a target cell or tissue to allow detection of the target cell or tissue.

In embodiments, increased (or improved) delivery (as compared with a delivery which is the same or similar except that it is carried out in the absence of an AHCM) can include one or more of:

increased delivery to, or amount or concentration in, the tumor vasculature, of the agent;

increased delivery to, or amount or concentration in, the tumor, e.g., the tumor vasculature interstitial matrix, of the agent;

increased delivery to, or amount or concentration in, in the tumor cells or tumor-associated cells (e.g., fibroblasts), of the agent;

increased flow rate, e.g., of the agent, in the tumor vasculature;

improved (or normalized) vasculature morphology (e.g., less tumor-like);

decompression of tumor vasculature;

increased pore size, or rate of diffusion of the agent, in the tumor, e.g., in the interstitial matrix;

increased perfusion of the agent, in the tumor, e.g., in the interstitial matrix;

broader and/or more homogeneous distribution of the agent throughout the tumor;

broader and/or more homogeneous distribution of the agent throughout the tumor interstitial matrix;

increased proportion of the agent in the tumor, e.g., the tumor interstitial matrix, as opposed to non-tumor tissue, e.g., peripheral blood;

inhibition of the TGF-beta pathway in the tumor, e.g., in the tumor vasculature interstitial matrix;

inhibition of the CTGF pathway in the tumor, e.g., in the tumor vasculature interstitial matrix;

inhibition of activity of the angiotension-II type-I receptor;

decrease in fibrosis, in the tumor, e.g., the tumor vasculature interstitial matrix;

decrease in the level or production of an extracellular matrix component, such as a fiber (e.g., collagen, procollagen), and/or a polysaccharide (e.g., a glycosaminoglycan such as hyaluronan or hyaluronic acid);

decrease in collagen or collagen deposition, in the tumor, e.g., the tumor vasculature interstitial matrix; or

decrease hyaluronan levels in the tumor, e.g., the tumor vasculature interstitial or stromal matrix.

In some embodiments, increased (or improved) delivery (as compared with a delivery which is the same or similar except that it is carried out in the absence of an AHCM) can also include increased amount of the agent distributed to at least a portion of the tumor. In some embodiments, the increased amount of the agent delivered to the tumor in the presence of the AHCM can be distributed homogenously or heterogeneously throughout the tumor.

“Efficacy” as used herein in the context of therapy, e.g., cancer therapy, can be characterizes as the extent to which a therapy has a desired effect, including but not limited to, alleviation of a symptom, diminishment of extent of disease, stabilized state of disease, delay or slowing of disease progression, amelioration or palliation of the disease state, and remission (whether partial or total), whether detectable or undetectable.

Improved efficacy, in the context of efficacy of cancer therapy, can be characterized by one or more of the following: an increase in an anti-tumor effect, of the cancer therapy, and/or a lessening of unwanted side effects (e.g., toxicity), of the cancer therapy, as compared with a treatment which is the same or similar except that it is carried out in the absence of treatment with an AHCM. In one embodiment, the increase in the anti-tumor effect of the cancer therapy includes one or more of: inhibiting primary or metastatic tumor growth; reducing primary or metastatic tumor mass or volume; reducing size or number of metastatic lesions; inhibiting the development of new metastatic lesions; reducing one or more of non-invasive tumor volume or metabolism; providing prolonged survival; providing prolonged progression-free survival; providing prolonged time to progression; and/or enhanced quality of life.

In some embodiments, the term “improved efficacy” as used herein, with respect to a cancer therapy in combination with an AHCM, can refer to an increase in reduction of primary or metastatic tumor growth by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to the reduction of primary or metastatic tumor growth during a cancer therapy alone (i.e., in the absence of an AHCM). In some embodiments, the administration of an ARCM in combination with a cancer therapy can increase the reduction of primary or metastatic tumor growth by at least about 1-fold, at least about 2-fold, at least about 3-fold, at least about 5-fold, at least about 6-fold, at least about 7-fold, or higher, as compared to the reduction of primary or metastatic tumor growth during a cancer therapy alone (i.e., in the absence of an AHCM). Methods for monitoring tumor growth in vivo are well known in the art, e.g., but not limited to, X-ray, CT scan, MRI and other art-recognized medical imaging methods.

In some embodiments, the term “improved efficacy” as used herein, with respect to a cancer therapy in combination with an AHCM, can refer to an increase in perfusion of an anti-cancer agent (e.g., low molecular weight therapeutics or nanotherapeutics such as DOXIL® or immune cells) into a tumor, e.g., by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to perfusion of an anti-cancer agent alone (i.e., in the absence of an ARCM). In some embodiments, the administration of an AHCM in combination with a cancer therapy can increase perfusion of an anti-cancer agent (e.g., low molecular weight therapeutics or nanotherapeutics such as DOXIL®) into a tumor, by at least about 1-fold, at least about 2-fold, at least about 3-fold, at least about 5-fold, at least about 6-fold, at least about 7-fold, or higher, as compared to the perfusion efficiency of an anti-cancer agent alone (i.e., in the absence of an AHCM). Methods to measure tumor perfusion in vivo are well established in the art, including, but not limited to, positron emission tomography (PET), and ultrasound or contrast-enhanced ultrasound.

In some embodiments, the term “improved efficacy” as used herein, with respect to a cancer therapy in combination with an AHCM, can refer to an increase in reduction in expression level of at least one biomarker, e.g., at least one cancer biomarker (e.g., in a biological sample such as a blood sample, a serum sample, a plasma sample or a tissue biopsy), e.g., by at least about 5%, at least about 10%, at least about 20%, at least about 30%, at least about 40%, at least about 50%, at least about 60%, at least about 70%, at least about 80%, at least about 90%, at least about 95%, up to and including 100%, as compared to the reduction in expression level of the at least one cancer biomarker when administered with a cancer therapy, alone (i.e., in the absence of an AHCM). In some embodiments, the administration of an ARCM in combination with a cancer therapy can increase the reduction in expression level of at least one biomarker (e.g., in a biological sample such as a blood sample, a serum sample, a plasma sample or a tissue biopsy) by at least about 1-fold, at least about 2-fold, at least about 3-fold, at least about 5-fold, at least about 6-fold, at least about 7-fold, or higher, as compared to the reduction in expression level of the at least one cancer biomarker when administered with a cancer therapy alone (i.e., in the absence of an AHCM). Examples of a biomarker in the serum, plasma or tissue can include, but are not limited to, TGF-beta 1, TGF-beta 2, CTGF, TSP-1, collagen I, collagen II, collagen III, or collagen IV. Expression levels of biomarkers can be measured on a transcript level and/or a protein level, using any art-recognized analytical methods, e.g., PCR, western blot, ELISA, and/or immunostaining.

“Blood pressure” is usually classified based on the systolic and diastolic blood pressures. “Systolic blood pressure” or Psys refers to the blood pressure in vessels during a heart beat. “Diastolic blood pressure” or Pdias refers to the pressure between heartbeats. A systolic or the diastolic blood pressure measurement higher than the accepted normal values for the age of the individual is classified as prehypertension or hypertension. A systolic or the diastolic blood pressure measurement lower than the accepted normal values for the age of the individual is classified as hypotension. A “normal” systolic pressure for an adult is typically in the range of 90-120 mmHg; a “normal” diastolic pressure is usually in the range of 60-80 mmHg. In the population, the average blood pressure (Psys/Pdias ratio) can range from 110/65 to 140/90 mmHg for an adult; 95/65 mmHg for a 1 year infant, and 100/65 mmHg for a 6-9 year old.

Hypertension has several subclassifications including, prehypertension (120/80 to 139/89 mmHg); hypertension stage I (140/90 to 159 to 99 mmHg), hypertension stage II (greater or equal to 160/100 mmHg, and isolated systolic hypertension (greater or equal to 140/90 mmHg). Isolated systolic hypertension refers to elevated systolic pressure with normal diastolic pressure and is common in the elderly. These classifications are made after averaging a patient's resting blood pressure readings taken on two or more office visits.

Hypertension is generally diagnosed on the basis of a persistently high blood pressure. Usually this requires three separate sphygmomanometer measurements at least one week apart. Often, this entails three separate visits to the physician's office. Initial assessment of the hypertensive patient should include a complete medical history and physical examination.

As used herein, “hypertension” or “high blood pressure,” refers to a prehypertensive or a hypertensive stage having a systolic pressure of 120 or greater (typically, 140 or greater) and a diastolic pressure of 80 or greater (all blood pressures herein are expressed as mmHg).

As used herein, the term “mean arterial pressure” (MAP) is art recognized and refers to the average over a cardiac cycle and is determined by the cardiac output (CO), systemic vascular resistance (SVR), and central venous pressure (CVP), MAP=(CO×SVR)+CVP. MAP can be approximately determined from measurements of the systolic pressure (Psys) and the diastolic pressure (Pdias), while there is a normal resting heart rate, MAP is approximately Pdias+⅓(Psys−Pdias).

“Anti-hypertensive agent,” as used herein refers to an agent (e.g., a compound, a protein) that when administered at a selected dose (referred to herein as “an anti-hypertensive dose”) reduces blood pressure, typically in a patient (e.g., a hypertensive patient). Anti-hypertensive agents are routinely used clinically to treat patients with high blood pressure at doses known in the art. Exemplary anti-hypertensive agents, include but are not limited to, renin angiotensin aldosterone system antagonists (“RAAS antagonists”), angiotensin converting enzyme (ACE) inhibitors, and angiotensin II receptor blockers (AT₁ blockers). Exemplary anti-hypertensive doses of some of these agents are also disclosed herein.

“Sub-anti-hypertensive dose,” as used herein, refers to a dose of an anti-hypertension agent that is typically less than the lowest dose that would be used to treat a patient for high blood pressure. In an embodiment, a sub-anti-hypertensive dose has one or more of the following properties:

it does not substantially lower blood pressure, e.g., the mean arterial blood pressure, of the subject, e.g., a hypertensive subject;

it reduces mean arterial blood pressure in the subject by less than 1%, 5%, 10%, 15%, 20%, 25%, 30%, 35%, 40%, 45%, 50%;

it reduces blood pressure by less than 1%, 5%, 10%, 15%, 20%, 25%, 30%, 35%, 40%, 45%, 50%, 60%, 70%, 80%, 90%, or less of the reduction caused by a standard of care anti-hypertensive dose for that AHCM;

it is less than 1%, 5%, 10%, 15%, 20%, 25%, 30%, 35%, 40%, 45%, 50%, 60%, 70%, 80%, 90% of the dose of that ARCM that would bring the subject's blood pressure into the normal range, e.g, 120 systolic and 80 diastolic, or a dose that would bring the subjects blood pressure into the range of to 120+/−5 systolic and 80+/−5 diastolic; or

it is less than a standard of care anti-hypertensive dose.

In certain embodiments, the ability of a dose to meet one or more of these standards can be made as measured after a preselected number of dosages, e.g., 1, 2, 5, or 10, or after sufficient dosages that a steady state level, e.g., plasma level, is attained.

An “ARCM,” as used herein, can be an agent having one or more of the following properties:

it is an antagonist of renin angiotensin aldosterone system (“RAAS antagonist”),

it is an angiotensin converting enzyme (ACE) inhibitor,

it is an angiotensin II receptor blocker (AT₁ blocker),

it is a thrombospondin 1 (TSP-1) inhibitor, it is a transforming growth factor beta 1 (TGF-β1) inhibitor,

it is an inhibitor of SDF-1a; or

it is a connective tissue growth factor (CTGF) inhibitor.

“Treating” a tumor, as used herein, typically refers to one or more of the following:

inhibiting primary or metastatic tumor growth;

reducing primary or metastatic tumor mass or volume;

reducing size or number of metastatic lesions;

inhibiting the development of new metastatic lesions;

reducing one or more of non-invasive tumor volume or metabolism;

providing prolonged survival;

providing prolonged progression-free survival;

providing prolonged time to progression; and/or enhanced quality of life.

Various aspects of the invention are described in further detail below. Additional definitions are set out throughout the specification.

Anti-Hypertensive and/or Collagen Modifying Agents (AHCM Agents)

In certain embodiments, the AHCM agent used in the methods and compositions of the invention can be chosen from one or more of: an antagonist of renin angiotensin aldosterone system (“RAAS antagonist”), an angiotensin converting enzyme (ACE) inhibitor, an angiotensin II receptor blocker (AT₁ blocker), a thrombospondin 1 (TSP-1) inhibitor, a transforming growth factor beta 1 (TGF-β1) inhibitor, and a connective tissue growth factor (CTGF) inhibitor. The method can include one, two, three or more AHCM agents, alone or in combination with one or more cancer therapeutics.

Exemplary antagonists of renin angiotensin aldosterone system (RAAS) include, but are not limited to, aliskiren (TEKTURNA®, RASILEZ®), remikiren (Ro 42-5892), enalkiren (A-64662), SPP635, and derivatives thereof.

Exemplary angiotensin converting enzyme (ACE) inhibitors include, but are not limited to, benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), and derivatives thereof.

Exemplary angiotensin II receptor blockers (AT₁ blockers) include, but are not limited to, losartan (COZAAR®), candesartan (ATACAND®), eprosartan mesylate (TEVETEN®), EXP 3174, irbesartan (AVAPRO®), L158,809, olmesartan (BENICAR®), saralasin, telmisartin (MICARDIS®), valsartan (DIOVAN®), and derivatives thereof.

In one embodiment, the AT₁ blocker is losartan, or a derivative thereof. Losartan is an anti-hypertensive agent with minimal safety risks (Johnston CI (1995) Lancet 346:1403-1407). Furthermore, in addition to its antihypertensive properties, losartan is also an antifibrotic agent that has been shown to reduce the incidence of cardiac and renal fibrosis (Habashi J P, et al. (2006) Science 312:117-121; and. Cohn R D, et al. (2007) Nat Med 13:204-210). The antifibrotic effects of losartan are caused, in part, by the suppression of active transforming growth factor-131 (TGF-β1) levels via an angiotensin II type I receptor (AGTR1) mediated down-regulation of TGF-β1 activators like thrombospondin-1 (TSP-1) (Habashi J P, et al. (2006) Science 312:117-121; Cohn R D, et al. (2007) Nat Med 13:204-210; Lavoie P, et al. (2005) J Hypertens 23:1895-1903; Chamberlain J S (2007) Nat Med 13:125-126; and Dietz H C (2010) J Clin Invest 120:403-407).

Exemplary thrombospondin 1 (TSP-1) inhibitors include, but are not limited to, ABT-510, CVX-045, LSKL, and derivatives thereof.

Exemplary transforming growth factor beta 1 (TGF-β1) inhibitors include, but are not limited to, CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, and derivatives thereof.

Exemplary connective tissue growth factor (CTGF) inhibitors include, but are not limited to, DN-9693, FG-3019, and compounds described in European Patent Application Publication No. 1839655, U.S. Pat. No. 7,622,454, and derivatives thereof.

Exemplary beta-blockers include, but are not limited to, atenolol (TENORMIN®), betaxolol (KERLONE®), bisoprolol (ZEBETA®), metoprolol (LOPRESSOR®), metoprolol extended release (TOPROL XI®), nadolol (CORGARD®), propranolol (INDERAL®), propranolo long-acting (INDERAL LA®), timolol (BLOCADREN®), acebutolol (SECTRAL®), penbutolol (LEVATOL®), pindolol, carvedilol (COREG®), labetalol (NORMODYNE®, TRANDATE®), and derivatives thereof.

In one embodiment, the ARCM agent is a TGF-β1 inhibitor, e.g., an anti-TGF-β1 antibody, a TGF-β1 peptide inhibitor. In certain embodiment, the TGF-β1 inhibitor is chosen from one or more of: CAT-192, fresolimumab (GC1008), LY 2157299, Peptide 144 (P144), SB-431542, SD-208, compounds described in U.S. Pat. No. 7,846,908 and U.S. Patent Application Publication No. 2011/0008364, or a derivative thereof.

Suitable doses for administration of the AHCM agent can be evaluated based on the standard of care anti-hypertensive doses of the AHCM agents are available in the art.

Exemplary standard of care anti-hypertensive and anti-heart failure doses and dosage formulations for AT₁ inhibitors in humans are as follows: 25-100 mg day⁻¹ of losartan (available in a dosage form for oral administration containing 12.5 mg, 25 mg, 50 mg or 100 mg of losartan); 4 to 32 mg day⁻¹ of candesartan (ATACAND®) (e.g., available in a dosage form for oral administration containing 4 mg, 8 mg, 16 mg, or 32 mg of candesartan); 400 to 800 mg day⁻¹ of eprosartan mesylate (TEVETEN®) (e.g., available in a dosage form for oral administration containing 400 or 600 mg of eprosartan); 150 to 300 mg day⁻¹ of irbesartan (AVAPRO®) (e.g., available in a dosage form for oral administration containing 150 or 300 mg of irbesartan); 20 to 40 mg day⁻¹ of olmesartan (BENICAR®) (available in a dosage form for oral administration containing 5 mg, 20 mg, or 40 mg of olmesartan); 20 to 80 mg day⁻¹ of telmisartin (MICARDIS®) (e.g., available in a dosage form for oral administration containing of 20 mg, 40 mg or 80 mg of telmisartin); and 80 to 320 mg day⁻¹ of valsartan (DIOVAN®) (e.g., available in a dosage form for oral administration containing 40 mg, 80 mg, 160 mg or 320 mg of valsartan).

Exemplary standard of care anti-hypertensive and anti-heart failure doses and dosage formulations for ACE inhibitors in humans are as follows: 10 to 40 mg day⁻¹ of benazepril (LOTENSIN®) (Lotensin (benazepril) is supplied as tablets containing 5 mg, 10 mg, 20 mg, or 40 mg of benazepril hydrochloride for oral administration); 25 to 100 mg day⁻¹ of captopril (CAPOTEN®) (available in a dosage form for oral administration containing 12.5 mg, 25 mg, 50 mg or 100 mg of captopril); 5 to 40 mg day⁻¹ of enalapril (VASOTEC®) (available in a dosage form for oral administration containing 2.5 mg, 5 mg, 10 mg or 20 mg of enalapril; 10 to 40 mg day⁻¹ of fosinopril (MONOPRIL®) (available in a dosage form for oral administration containing 10 mg, 20 mg, or 40 mg of fosinopril); 10 to 40 mg day⁻¹ of lisinopril (PRINIVIL®, ZESTRIL®) (available in a dosage form for oral administration containing 2.5 mg, 5 mg, 10 mg, 20 mg, 30 mg or 40 mg of lisinopril); 7.5 to 30 mg day⁻¹ of moexipril (UNIVASC®) (available in a dosage form for oral administration containing 7.5 mg or 15 mg of Moexipril); 4 to 8 mg day⁻¹ of perindopril (ACEON®) (available in a dosage form for oral administration containing 2 mg, 4 mg or 8 mg of perindopril), 10 to 80 mg day⁻¹ of quinapril (ACCUPRIL®) (available in a dosage form for oral administration containing 5 mg, 10 mg, 20 mg, or 40 mg of quinapril); 2.5 to 20 mg day⁻¹ of ramipril (ALTACE®) (available in a dosage form for oral administration containing 1.25 mg, 2.5 mg, 5 mg, or mg of ramipril); 1 to 4 mg day⁻¹ of trandolapril (MAVIK®) (available in a dosage form for oral administration containing 1 mg, 2 mg, or 4 mg of trandolapril).

In one embodiment, the AHCM agent is administered at a standard of care anti-hypertensive and anti-heart failure doses and dosage formulations, e.g., a dose or dosage formulation as described herein.

In certain embodiments, a sub-anti-hypertensive dose or dosage formulation of the AHCM agent is desirable, e.g., a dose of the AHCM agent that is less than the standard of care dose or dosage formulation. In one embodiment, the sub-anti-hypertensive dose or dosage formulation has a minimal effect in blood pressure in a hypertensive subject (e.g., decreases the mean arterial blood pressure in a hypertensive subject by less than 20%, 10%, or 5% or less). In certain embodiments, the AHCM agent is administered at a dose or dosage formulation that is less than 0.01, 0.05, 0.1, 0.2, 0.3, 0.4, 0.5, 0.6, 0.7, 0.8, 0.9, that of the standard of care anti-hypertensive dose (e.g., the lower standard of care dose). In one embodiment, the dose or dosage formulation is in the range of, for example, 0.01-0.9-fold, 0.02-0.8-fold, 0.05-0.7-fold, 0.1-0.5 fold, 0.1-0.2-fold, that of the standard of care dose or dosage formulation for anti-hypertensive or anti-heart failure use. Standard of care doses or dosage formulation of the AHCM are available in the art, some of which are exemplified herein.

In yet other embodiments, the AHCM agent is administered at a dose or dosage formulation that is greater than the standard of care dose or dosage formulation for anti-hypertensive or anti-heart failure use (e.g., a dose or dosage form that is greater than 1.1, 1.5, 1.7, 2, 3, 4, 5, 10-fold or higher, that of the standard of care dose for anti-hypertensive or anti-heart failure use). In one embodiment, the dose or dosage formulation is in the range of, for example, 1.1 to 10-fold, 1.5-5-fold, 1.7 to 4-fold, or 2-3-fold, that of the standard of care dose or dosage formulation for anti-hypertensive or anti-heart failure use. Standard of care doses or dosage formulation of the AHCM are available in the art, some of which are exemplified herein.

The standard of care dose and dosage forms are provided herein for a number of ARCMs, e.g., losartan. In an embodiment, the dose and/or dosage form is less than (or higher than) the standard of care dose and/or dosage form. In an exemplary embodiment, it is less than 0.01, 0.02, 0.05, 0.1, 0.2, 0.5, 0.7, 0.8, 0.9-fold, that of the standard of care dose or dosage form. In embodiments, the dose or dosage form contains an amount of AHCM that is within a range of the reduced amounts of the standard of care dose and/or dosage form. E.g., an AHCM dosage form that is 0.01-0.9-fold, 0.02-0.8-fold, 0.05-0.7-fold, 0.1-0.5 fold, 0.1-0.2-fold, that of the standard of care dose or dosage form. In certain embodiments, the range of the dose or the dosage form is 0.5-2.0 times a reduced dose or dosage form recited herein, so long as the dose or dosage form value is less than the standard of care dose or dosage form. By way of example, a standard of care dosage form for losartan is 12.5 mg. Thus, in embodiments, the dosage form is 0.125 mg (0.01×12.5 mg); 0.625 mg (0.05×12.5 mg); 1.25 mg (0.1×12.5 mg); 2.5 mg (0.2×12.5 mg); or 6.25 mg (0.5×12.5 mg). In an embodiment, the AHCM dosage form is in the range 0.5-2.0 (0.125 mg)=0.0625-0.25 mg; 0.5-2.0 (0.625 mg)=0.312-1.25 mg; and so on, so long as the dose or dosage form value is less than the standard of care dose or dosage form. This calculation can be applied to any standard of care dose and/or dosage form for any AHCM described herein. In certain embodiment, the value is less than the standard of care values. In other embodiments, the value is greater than the standard of care values.

In one embodiment, the dose of the AHCM agent is calculated based on the severity of the fibrosis in the tumor sample.

In some embodiments, the dose of the AHCM agent can be a sub-anti-hypertensive dose, which does not have any anti-tumor effect, e.g., no significant effect on inhibiting or preventing tumor growth or progression when administered alone. In some embodiments, the dose of the AHCM agent can be comparable to or greater than the standard of care dose or dosage formulation for anti-hypertensive or anti-heart failure use, and does not have any anti-tumor effect, e.g., no significant effect on inhibiting or prevening tumor growth or progression when administered alone.

### Therapeutic Methods

In one aspect, the invention relates to a method of treating a disorder, e.g., a hyperproliferative disorder (e.g., a cancer) by administering to a patient an AHCM agent, alone or in combination with a therapy or a therapeutic agent, e.g., an anti-cancer agent as described herein.

As used herein, and unless otherwise specified, the terms “treat,” “treating” and “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

For example, in the case of treating cancer, in some embodiments, therapeutic treatment can refer to inhibiting or reducing tumor growth or progression after administration in accordance with the methods or administration with the pharmaceutical compositions described herein. For example, tumor growth or progression is inhibited or reduced by at least about 10%, at least about 15%, at least about 20%, at least about 30%, at least about 40%, or at least about 50%, after treatment. In another embodiment, tumor growth or progression is inhibited or reduced by more than 50%, e.g., at least about 60%, or at least about 70%, after treatment. In one embodiment, tumor growth or progression is inhibited or reduced by at least about 80%, at least about 90% or greater, as compared to a control (e.g. in the absence of the pharmaceutical composition described herein).

In another embodiment, the therapeutic treatment refers to alleviation of at least one symptom associated with cancer. Measurable lessening includes any statistically significant decline in a measurable marker or symptom, such as measuring a cancer biomarker, such as serum/plasma cancer biomarker in a blood sample, after treatment. In one embodiment, at least one cancer biomarker or sympton is alleviated by at least about 10%, at least about 15%, at least about 20%, at least about 30%, at least about 40%, or at least about 50%. In another embodiment, at least one cancer biomarker or sympton is alleviated by more than 50%, e.g., at least about 60%, or at least about 70%. In one embodiment, at least one cancer biomarker or sympton is alleviated by at least about 80%, at least about 90% or greater, as compared to a control (e.g. in the absence of the pharmaceutical composition described herein).

As used herein, unless otherwise specified, the terms “prevent,” “preventing” and “prevention” contemplate an action that occurs before a patient begins to suffer from the regrowth of the cancer and/or which inhibits or reduces the severity of the cancer.

As used herein, and unless otherwise specified, a “therapeutically effective amount” of a compound is an amount sufficient to provide a therapeutic benefit in the treatment of the disorder (e.g., cancer), or to delay or minimize one or more symptoms associated with the disorder (e.g., cancer). A therapeutically effective amount of a compound means an amount of therapeutic agent, alone or in combination with other therapeutic agents, which provides a therapeutic benefit in the treatment or management of the disorder. The term “therapeutically effective amount” can encompass an amount that improves overall therapy, reduces or avoids symptoms or causes of the disorder (e.g., cancer), or enhances the therapeutic efficacy of another therapeutic agent.

As used herein, and unless otherwise specified, a “prophylactically effective amount” of a compound is an amount sufficient to prevent a disorder (e.g., regrowth of the cancer, or one or more symptoms associated with the cancer, or prevent its recurrence). A prophylactically effective amount of a compound means an amount of the compound, alone or in combination with other therapeutic agents, which provides a prophylactic benefit in the prevention of the disorder. The term “prophylactically effective amount” can encompass an amount that improves overall prophylaxis or enhances the prophylactic efficacy of another prophylactic agent.

As used herein, the term “patient” or “subject” refers to an animal, typically a human (i.e., a male or female of any age group, e.g., a pediatric patient (e.g, infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug. The methods and/or pharmaceutical compositions described herein can also be used to treat domesticated animals or pets such as cats and dogs.

As used herein, “cancer” and “tumor” are synonymous terms.

As used herein, “cancer therapy” and “cancer treatment” are synonymous terms.

As used herein, “chemotherapy,” “chemotherapeutic,” “chemotherapeutic agent” and “anti-cancer agent” are synonymous terms.

In some embodiments, the AHCM agent, alone or in combination, is a first line treatment for the cancer, i.e., it is used in a subject who has not been previously administered another drug intended to treat the cancer.

In other embodiments, the AHCM agent, alone or in combination, is a second line treatment for the cancer, i.e., it is used in a subject who has been previously administered another drug intended to treat the cancer.

In other embodiments, the AHCM agent, alone or in combination, is a third or fourth line treatment for the cancer, i.e., it is used in a subject who has been previously administered two or three other drugs intended to treat the cancer.

In some embodiments, the AHCM agent is administered to a subject before, during, and/or after radiation or surgical treatment of the cancer.

In some embodiments, the AHCM agent is administered, alone or in combination with a cancer therapy or an anti-cancer agent, to a subject who previously did not respond to at least one cancer therapy or anti-cancer agent, including at least two, at least three, or at least four cancer therapies or anti-cancer agents. In such embodiments, the AHCM agent can be administered to a subject in combination with the cancer therapy or anti-cancer agent to which he/she previously did not respond, or in combination with a cancer therapy or anti-cancer agent different from the one(s) he/she has been treated with.

In other embodiments, the AHCM agent is administered as adjunct therapy, i.e., a treatment in addition to primary therapy. In embodiments, the adjuvant effect of the AHCM administered in combination with a primary therapy can be additive.

### Disorders

The AHCM, alone or in combination with a microenvironment modulator and/or a therapy or a therapeutic agent, e.g., an anti-cancer agent as described herein can be used to treat or prevent a disorder, e.g., a hyperproliferative disorder (e.g., a cancer).

In certain embodiments, the disorder is chosen from one or more of a hyperproliferative disorder, a cancer, a fibrotic disorder, an iinflammatory disorder or an autoimmune disorder.

In certain embodiments, the cancer is an epithelial, mesenchymal or hematologic malignancy In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

By “hyperproliferative cancerous disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

As used herein, the terms “cancer,” “tumor” or “tumor tissue” refer to an abnormal mass of tissue that results from excessive cell division, in certain cases tissue comprising cells which express, over-express, or abnormally express a hyperproliferative cell protein. A cancer, tumor or tumor tissue comprises “tumor cells” which are neoplastic cells with abnormal growth properties and no useful bodily function. Cancers, tumors, tumor tissue and tumor cells may be benign or malignant. A cancer, tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In other embodiments, the AHCM agent, as described above and herein, is used to treat a hyperproliferative disorder, e.g., a hyperproliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis.

In other embodiment, the subject treated has a hyperproliferative genetic disorder, e.g., a hyperproliferative genetic disorder chosen from Marfan's syndrome or Loeys-Dietz syndrome. Losartan has been shown to treat human Marfan syndrome, a connective tissue disorder caused by mutations in the gene that encodes the extracellular matrix protein, fibrillin-1 (Dietz, H. C. et al. (2010) New Engl J Med 363(9):852-863). Fibrillin-1 comprises the microfibrils of elastic tissue and a component of many other connective tissues. Affected patients with Marfan syndrome have blood vessel abnormalities such as aortic aneurysms. The vascular disease can result in blood vessel rupture and death in childhood and later in life. Dietz et al. first found in mouse models of Marfan syndrome that excessive activation of latent TGF-β has an important role in the pathophysiology. They used losartan in the affected mice and showed striking effects in improving blood vessel architecture and prevented the development of aortic aneurysms. They have also used losartan to treat children with Marfan syndrome and demonstrated that the drug can strikingly prevent progression of aortic and muscular lesions. Aortic diseases other than Marfan syndrome can also benefit from the use of losartan. Inhibition of activation of latent TGF-β locally and decreasing circulating levels of active TGF-β thus can have effects on components of connective tissues other than collagen in the extracellular matrix of cancer tissues that alter delivery and efficacy of nanotherapeutics.

In other embodiments, the hyperproliferative disorder (e.g., the hyperproliferative fibrotic disorder) is chosen from one or more of chronic obstructive pulmonary disease, asthma, aortic aneurysm, radiation-induced fibrosis, skeletal-muscle myopathy, diabetic nephropathy, and/or arthritis.

Additional exemplary hyperproliferative disorders that can be treated by the methods and compositions of the invention are disclosed in Sounni, N. E. et al. (2010) Diseases Models & Mechanisms 3:317-332.

In yet other embodiments, the disorder is chosen from an inflammatory or an autoimmune disorder chosen from multiple sclerosis, inflammatory bowel disease, scleroderma, lupus, rheumatoid arthritis or osteoarthritis.

In certain embodiments, the inflammatory disorder is an inflammatory disorder of: the gastrointestinal tract or a gastrointestinal organ, e.g., colitis, Crohn's disease, inflammatory bowel disease (IBD), Barrett's esophagus and chronic gastritis; the lung (e.g., asthma, chronic obstructive pulmonary disease (COPD); the skin (e.g., psoriasis), the cardiovascular system (e.g., atherosclerosis, cholesterol metabolic disorders, oxygen free radical injury, ischemia), the nervous system (e.g., Alzheimer's disease, multiple sclerosis), liver (e.g., hepatitis), kidney (e.g., nephritis), and the pancreas (e.g., pancreatitis).

In other embodiments, the inflammatory disorder is associated with an autoimmune disorder, e.g., arthritis (including rheumatoid arthritis, juvenile rheumatoid arthritis, osteoarthritis, psoriatic arthritis, lupus-associated arthritis, autoimmune thyroiditis or ankylosing spondylitis); scleroderma; lupus; systemic lupus erythematosis; HIV; Sjogren's syndrome; vasculitis; multiple sclerosis; dermatitis (including atopic dermatitis and eczematous dermatitis), myasthenia gravis, inflammatory bowel disease (IBD), Crohn's disease, colitis, diabetes mellitus (type I); acute inflammatory conditions (e.g., endotoxemia, sepsis and septicaemia, toxic shock syndrome and infectious disease); transplant rejection and allergy.

### Combination Therapy

It will be appreciated that the AHCM agent, as described above and herein, can be administered in combination with one or more additional therapies, e.g., such as radiation therapy, PDT, surgery, immune therapy, and/or in combination with one or more therapeutic agents, to treat the cancers described herein.

By “in combination with,” it is not intended to imply that the therapy or the therapeutic agents must be administered at the same time and/or formulated for delivery together, although these methods of delivery are within the scope of the invention. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination will be lower than those utilized individually.

In certain embodiments, the AHCM and/or the therapy (e.g., the cancer or hyperproliferative therapy) is administered in combination with a microenvironment modulator. The combined administration of the AHCM and the microenvironment modulator can be used to enhance the efficacy (e.g., penetration and/or diffusion), of a therapy, e.g., a cancer therapy, in a tumor or tumor vasculature in a subject. Such combination may cause one or more of: reduce solid stress (e.g., growth-induced solid stress in tumors); decrease tumor fibrosis; reduce interstitial hypertension or interstitial fluid pressure (IFP); increase interstitial tumor transport; increase tumor or vessel perfusion; increase vascular diameters and/or enlarge compressed or collapsed blood vessels; reduce or deplete one or more of: cancer cells, or stromal cells (e.g., tumor associated fibroblasts or immune cells); decrease the level or production of extracellular matrix components, such as fibers (e.g., collagen, procollagen), and/or polysaccharides (e.g., glycosaminoglycans such as hyaluronan or hyaluronic acid); decrease the level or production of collagen or procollagen; decreases the level or production of hyaluronic acid; increases tumor oxygenation; decreases tumor hypoxia; decreases tumor acidosis; enables immune cell infiltration; decreases immunosuppression; increases antitumor immunity; decreases cancer stem cells (also referred to herein as tumor initiating cells), thereby enhacing the penetration and/or distribution of the therapy, e.g., the cancer therapy.

Exemplary microenvironment modulators are disclosed herein, and include, but are not limited to, an anti-angiogenic therapy, for example, an inhibitor of vascular endothelial growth factor (VEGF) pathway; an agent that decreases the level or production of hyaluronic acid; an inhibitor of the hedgehog pathway; an agent that improves drug penetration in tumors. In one embodiment, the agent is a disulfide-based cyclic RGD peptide peptide (iRGD) or an analogue thereof; a taxane therapy (e.g., taxane-induced apoptosis); an agent that decreases the level or production of collagen or procollagen; an anti-fibrotic agent and/or a profibrotic pathway inhibitor.

In one embodiment, the microenvironment modulator includes an anti-angiogenic therapy, for example, an inhibitor of vascular endothelial growth factor (VEGF) pathway. Exemplary VEGF pathway inhibitors include, but are not limited to, an antibody against VEGF (e.g., bevacizumab); a VEGF receptor inhibitor (e.g., an inhibitor of VEGFR-1 inhibitor, a VEGFR-2 inhibitor, or a VEGFR-3 inhibitor (e.g., VEGFR inhibitors such as Cediranib (AZD2171)); a VEGF trap (e.g., a fusion protein that includes a VEGFR domain (e.g., a VEGFR1 domain 2 and a VEGFR2 domain 3) fused to an Fc fragment of an IgG); and an anti-VEGF aptamer (or a pegylated derivative thereof (e.g., MACUGEN®).

In another embodiment, the microenvironment modulator includes an agent that decreases the level or production of hyaluronic acid (HA). Enzymatic targeting of the stroma using systemic administration of a pegylated derivative of hyaluronidase (PEGPH20) has been shown to ablate stromal HA in a model for pancreatic ductal adenocarcinoma (PDA) and increase vessel diameter in pancreatic tumors; hyaluronidase derivatives, in combination with standard chemotherapeutic agents (e.g., gemcitabine), can remodel the tumor microenvironment and increase overall survival (see e.g., Provenzano, P. et al. (2012) Cancer Cell 21: 418-429). Thus, combined administration of the AHCM and the microenvironment modulator can be used to enhance penetration and/or diffusion of a cancer therapy in a tumor or tumor vasculature, by for example, decreasing certain matrix components, e.g., HA, in the stroma. Exemplary HA-depleting agents include, but are not limited to, an anti-hyaluronan enzymatic therapy such as hyaluronidase or a derivative thereof (e.g., pegylated recombinant human hyaluronidase) (e.g., PH20, PEGPH20); and an antibody against hyaluronic acid.

In another emdodiment, the microenvironment modulator includes an inhibitor of the hedgehog pathway. Hedgehog inhibitors have been shown to increase vessel density in pancreatic tumors (Olive, K. P. et al. (2009) Science 324:1457-61), presumably by reducing stromal cell density and solid stress. Exemplary hedgehog inhibitors include, but are not limited to, IPI-926, GDC-0449, cylopamine or an analogue thereof, and GANT58.

In another embodiment, the microenvironment modulator includes an agent that improves drug penetration in tumors. In one embodiment, the agent is a disulfide-based cyclic RGD peptide peptide (iRGD) or an analogue thereof (e.g., described in Sugahara, K N et al. (2010) Science 328:1031-5; Ye, Y. et al. (2011) Bioorg Med Chem. Lett. 21(4):1146-50).

In yet another embodiment, the microenvironment modulator includes a taxane therapy (e.g., taxane-induced apoptosis as described in Griffon-Etienne, G. et al. (1999) Cancer Res. 59(15):3776-82).

In another embodiment, the microenvironment modulator includes an agent that modulates (e.g, inhibits) a hypoxia inducible factor (HIF), for example, an agent that inhibits hypoxia-inducible factors 1α and 2α (HIF-1α and HIF-2α). HIF activity has been shown to be involved in inflammation (e.g., rheumatoid arthritis) and angiogenesis associated with cancer tumor growth. HIF inhibitors, such as phenethyl isothiocyanate (PEITC) are under investigation for anti-cancer effects (Syed Alwi S S, et al. (2010) Br. J. Nutr. 104 (9): 1288-96; Semenza G L (2007). Drug Discov. Today 12 (19-20): 853-9; Melillo G (2006). Mol. Cancer. Res. 4 (9): 601-5. In one embodiment, the agent is an antibody against an HIF. In another embodiment, the agent is an HIF chemical inhibitor, such as phenethyl isothiocyanate (PEITC).

In another embodiment, the microenvironment modulator includes an agent that decreases the level or production of collagen or procollagen. For example, an agent that degrades collagen, e.g., collagenase.

In one embodiment, the AHCM and/or the therapy (e.g., the cancer or hyperproliferative therapy) is administered in combination with a microenvironment modulator chosen from an anti-fibrotic agent or an inhibitor of a profibrotic pathway (a “profibrotic pathway inhibitor”) (e.g., a pathway dependent- or independent of TGF-beta and/or CTGF activation). In one embodiment, the AHCM and/or the cancer therapy is administered in combination with one or more of: an inhibitor of endothelin-1, PDGF, Wnt/beta-catenin, IGF-1, TNF-alpha, and/or IL-4. In another embodiment, the AHCM and/or the cancer therapy is administered in combination with an inhibitor of endothelin-1 and/or PDGF. In other embodiments, the AHCM and/or the cancer therapy is administered in combination with an inhibitor of one or more of chemokine receptor type 4 (CXCR4) (e.g., AMD3100, MSX-122); stromal-derived-factor-1 (SDF-1) (e.g., tannic acid); hedgehog (e.g., IPI-926, GDC-0449, cylopamine or an analogue thereof, or GANT58).

In certain embodiments, an inhibitor of a CXCR4 receptor and/or its ligand, SDF-1, is administered in combination with a therapy (e.g., a cancer or hyperproliferative therapy as described herein). Certain embodiments may further include administration of a further AHCM and/or a microenvironment modulator as described herein. Without wishing to be bound by theory, inhibition of CXCR4 receptor and/or its ligand, SDF-1, alone or in combination with an AHCM, e.g., an angiotensin II receptor blocker, can be used to reduce the desmoplasia in certain fibrotic or desmoplastic cancers, e.g., a fibrotic or a desmoplastic solid tumor, such as pancreatic cancers (e.g., pancreatic ductal adenocarcinoma (PDAC)). For example, activation of SDF-1a/CXCR4 and angiotensin II (ATII) signaling pathways is known to promote carcinoma activated fibroblasts (CAF) recruitment, activation, and matrix production in PDAC. Hypoxia, which is associated with PDAC, can induce SDF-1a and CXCR4 expression in cancer cells and CAFs through HIF-1a activation (Schioppa, T., et al. (2003) J Exp Med, 198: 1391-1402) while promoting growth and metastasis (Chang, Q., et al. (2011) Cancer Research, 71: 3110-3120). These effects arise, at least in part, through SDF-1a/CXCR4-dependent activation of CAFs (Gao, Z. et al. (2010) Pancreatology 10: 186-193; Moriyama, T. et al. (2010) Cancer 116: 3357-3368) and a CD133+/CXCR4+ cancer stem cell population (Hermann, P. C. et al. (2007) Cell Stem Cell 1: 313-323), which also confers chemoresistance (Singh, S. et al. (2010) Br J Cancer 103: 1671-1679). High SDF-1a levels (Liang, J. J., et al. (2010) Cancer Epidemiology Biomarkers & Prevention 19: 2598-2604) and CXCR4 levels (Marechal, R. et al. (2009) Br J Cancer, 100: 1444-1451) can be predictive of poor prognosis in PDAC patients. On the other hand, ATII signaling can stimulate CAF proliferation (Hama, K. et al. (2006) Biochemical and Biophysical Research Communications, 340: 742-750; Hama, K. et al. (2004) Biochem Biophys Res Commun. 315: 905-911; Shimizu, K. et al. (2008) J Gastroenterol Hepatol, 23 Suppl 1: S119-121), and ATII signaling through ATII-receptor type 1 (AT1) can stimulate CAF matrix production via TGF-β1 and ERK-dependent mechanisms (Rodriguez-Vita, J. et al. (2005) Circulation 111: 2509-2517; Yang, F. et al. (2009) Hypertension, 54: 877-884). ATII also induces TGF-β1 (Elenbaas, B. and Weinberg, R. A. (2001) Experimental Cell Research, 264: 169-184) and SDF-1a (Chu, P. Y. et al. (2010) Am J Pathol, 176: 1735-1742) expression by both cancer cells and CAFs, which can promote CAF proliferation and matrix production. Thus, inhibition of a CXCR4 receptor and/or its ligand, SDF-1, can be used (alone or with an inhibitor of ATII signaling) to enhance the distribution of a therapy in fibrotic or desmoplastic cancers.

Exemplary SDF-1/CXCR4 inhibitors that can be used include, but are not limited to, 2,2′-bicyclam; 6,6′-bicyclam; AMD3100 (IUPAC name: 1,1′-[1,4-phenylene-bis(methylene)]-bis-1,4,8,11-tetraazacyclotetradecane), as described in e.g., U.S. Pat. Nos. 5,021,409, 6,001,826 and 5,583,131; Plerixa for (trade name: Mozobil; IUPAC name: 1,1′-[1,4-Phenylenebis(methylene)]bis[1,4,8,11-tetraazacyclotetradecane); CXCR4 peptide inhibitors or analogs, e.g., T-140 analogs (e.g., 4F-benzoyl-TN14003, TC14012, TE14011, TC14003), CTCE-0214; CTCE-9908; and CP-1221, as well as other inhibitors such as antibodies against SDF-1 or CXCR4, RNA inhibitors (e.g., antisense, siRNAs), among others. Exemplary inhibitors are described in, for example, Tamamura, H. et al. Org. Biomol. Chem. 1:3656-3662, 2003; FEBS Letter 550:1-3 (2003): 79-83; Wong, D. et al. (2008) Clin. Cancer Res. 14(24): 7975-7980; US Patent Publications 2010/0055088; 2009/0221683; 2004/0209921, 2005/0059702, 2005/0043367, 2005/0277670, 2010/0178271, and 2003/0220341; U.S. Pat. Nos. 5,021,409, 6,001,826, 5,583,131, and Patent Publications WO 03/011277, WO 01/85196; WO 99/50461; WO 01/94420; WO 03/090512, each of which is incorporated herein by reference in their entirety.

In another embodiment, the AHCM and/or the cancer therapy is administered in combination with an anti-fibrotic agent, for example, a pirfenidone. Pirfenidone (PFD or 5-methyl-1-phenyl-2(1H)-pyridone, commercially available from Marnac, Inc.) is an agent that is being investigated for use in patients with pulmonary fibrosis. Pirfenidone has been shown to produce anti-fibrotic effects in several organs such as the heart, liver, lung and kidney. For example, PFD has been shown to have an inhibitory effect on fibroblast growth and collagen synthesis by reducing expression of profibrotic cytokines such as TGF-b (Iyer, S, N. et al. (2000) Inflammation 24:477-491). PFD has also been shown to reduce leiomyoma cell proliferation and collagen production in cultured cells, as well as reduce TGF-b expression in human malignant glioma cells (see e.g., Byung-Seok, L. et al. (1998) J of Clinical Endocrinology and Metabolism 83(1): 219-223; and Burghardt, I. et al. (2007) Biochem and Biophys Res. Comm. 354:542-547).

In other embodiments, the AHCM and/or the microenvironment modulator is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCl-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRAT™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the AHCM agent and/or the microenvironment modulator is administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the AHCM and/or the microenvironment modulator is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/ASO4 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA (6D)/TRICOM vaccine; recombinant vaccinia-CEA (6D)-TRICOM vaccine, recombinant modified vaccinia Ankara-5T4 vaccine, recombinant fowlpox-TRICOM vaccine, oncolytic herpes virus NV 1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/ASO4, fowlpox virus vaccine vector, vaccinia-tyrosinase vaccine, MEDI-517 HPV-16/18 VLP ASO4 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the AHCM and/or the microenvironment modulator is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the AHCM agent and/or the microenvironment modulator is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02. Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRONT™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADURT™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVAC), everolimus (AFINITOR®), gefitinib (IRESSA®), imatinib mesylate (STI-571, GLEEVECT™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the AHCM agent and/or the microenvironment modulator is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the AHCM and/or the microenvironment modulator is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

It will be appreciated that anti-tumor antibodies labeled with isotopes have been used successfully to destroy cells in solid tumors, as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

It will also be appreciated that, in accordance with the teachings herein, binding molecules can be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

In other embodiments, the cancer therapy includes an immune therapy used in combination with the AHCM, other cancer therapies, and/or the microenvironment modulator, described herein. Without wishing to be bound by theory, hypoxia and/or limited perfusion are believed to cause immunosuppression and/or limit the efficacy of certain immune therapies. AHCM, alone or in combination with therapies described herein can be used to improve the efficacy of said immune therapies. Examples of immune therapies include, but are not limited to, CTLA-4 blockade (e.g., an anti-14371236.1 CTLA-4 antibody (e.g., ipilimumab)); immune-based therapies (including, e.g., immune or dendritic cell-based vaccines and antagonists of immune inhibitory signals or checkpoints); cancer vaccines, e.g., Sipuleucel-T (APC8015, trade name Provenge, manufactured by Dendreon Corporation) is a therapeutic cancer vaccine for prostate cancer (CaP)); and adoptive T-cell-based therapies. Exemplary immune-based therapies include, but are not limited to, e.g., immune or dendritic cell-based vaccines (Seton-Rogers, S. (2012) Nature Reviews Cancer 12:230-231; Palucka, K. et al. (2012) Nature Reviews Cancer 12:265-277); effector memory CD8+ T cells (Bird, L. (2012) Nature Reviews Immunology 12:227); engineered tumor cells to activate Toll like Receptors (TLRs) and NOD-like Receptors (NLRs) (Leavy, O. (2012) Nature Reviews Immunology 12:227); antagonists of immune inhibitory signals or checkpoints (Pardoll, D. M. (2012) Nature Reviews Cancer 12:252-264).

In yet other embodiments, the cancer therapy includes PDT used in combination with the AHCM, other cancer therapies, and/or the microenvironment modulator, described herein. In certain embodiments, PDT includes administration of a photosensitizing agent (e.g., a porhyrin, a porpyrin precursor, a chorlin, or a phthalocyanine) followed by irradiation at a wavelength corresponding to an absorbance band of the sensitizer. In the presence of oxygen, a series of events lead to one or more of: cell death (e.g., tumor cell death), damage to the microvasculature, or induction of a local inflammatory reaction). PDT is reviewed in, e.g., Agostinis, P. et al. (2011) CA Cancer J. Clin. 61:250-281.

In other embodiments, the cancer therapy includes an inhibitor of a cancer stem cell (also referred to herein as a “cancer initiating cell”), used in combination with the AHCM, other cancer therapies and/or the microenvironment modulator, described herein. Without wishing to be bound by theory, hypoxia and cancer drugs (including anti-angiogenic drugs) and radiation therapy are believed to increase the number of cancer stem cells. AHCM, alone or in combination with, e.g., an inhibitor of a cancer stem cell, can be used to reduce the production of these stem cells. Exemplary inhibitors of cancer stem cells that can be used in combination include, but are not limited to, hedgehog (e.g., SMO) antagonists; and Wnt pathway antagonists (e.g., antibody, OMP-18R5).

In certain embodiments, the AHCM agent, the microenvironment modulator and/or the additional anti-cancer agent are administered concurrently (e.g., administration of the two agents at the same time or day, or within the same treatment regimen) and/or sequentially (e.g., administration of one agent over a period of time followed by administration of the other agent for a second period of time, or within different treatment regimens).

In one embodiment, the AHCM and/or the microenvironment modulator is administered prior to the anti-cancer agent. In other embodiments, the AHCM and/or the microenvironment modulator is administered prior to the anti-cancer agent, and followed by concurrent administration of the AHCM, the microenvironment modulator and/or the anti-cancer agent.

In certain embodiments, the AHCM agent, the microenvironment modulator and/or and the additional anti-cancer agent are administered concurrently. For example, in certain embodiments, the AHCM agent, the microenvironment modulator and/or and the additional anti-cancer agent are administered at the same time, on the same day, or within the same treatment regimen. In certain embodiments, the ARCM agent and/or the microenvironment modulator is administered before the additional anti-cancer agent on the same day or within the same treatment regimen.

In certain embodiments, the AHCM agent and/or the microenvironment modulator is concurrently administered with additional anti-cancer agent for a period of time, after which point treatment with the additional anti-cancer agent is stopped and treatment with the AHCM agent continues.

In other embodiments, the AHCM agent and/or the microenvironment modulator is concurrently with the additional anti-cancer agent for a period of time, after which point treatment with the ARCM agent and/or the microenvironment modulator is stopped and treatment with the additional anti-cancer agent continues.

In certain embodiments, the AHCM agent, the microenvironment modulator and/or the additional anti-cancer agent are administered sequentially. For example, in certain embodiments, the AHCM agent is administered after the treatment regimen of the additional anti-cancer agent and/or microenvironment modulator has ceased. In certain embodiments, the additional anti-cancer agent is administered after the treatment regimen of the AHCM agent and/or microenvironment modulator has ceased.

In some embodiments, the AHCM agent, microenvironment modulator and/or the anti-cancer agent can be administered in a pulse administration. In other embodiments, they can be administered as a pulse-chase administration, e.g., where an AHCM agent is administered for a brief period of time (pulse), followed by administration of an anti-cancer agent for a longer period of time (e.g., chase), or vice versa.

### Diagnostic Methods and Assays

ARCM agents can be used to improve diagnosis, treatment, prevention and/or prognosis of cancers in mammals, preferably humans. These diagnostic assays can be performed in vivo or in vitro, such as, for example, on blood samples, biopsy tissue or autopsy tissue.

Thus, the invention provides a diagnostic method useful during diagnosis of a cancer, which involves measuring the expression level of target protein or transcript in tissue or other cells or body fluid from an individual and comparing the measured expression level with a standard target expression levels in normal tissue or body fluid, whereby an increase in the expression level compared to the standard is indicative of a disorder.

One embodiment provides a method of detecting the presence of abnormal hyperproliferative cells, e.g., precancerous or cancerous cells, in a fluid or tissue sample, comprising assaying for the expression of the target in tissue or body fluid samples of an individual and comparing the presence or level of target expression in the sample with the presence or level of target expression in a panel of standard tissue or body fluid samples, where detection of target expression or an increase in target expression over the standards is indicative of aberrant hyperproliferative cell growth.

One aspect of the invention is a method for the in vivo detection or diagnosis of a cancer in a subject, preferably a mammal and most preferably a human. In one embodiment, diagnosis comprises: a) administering (for example, parenterally, subcutaneously, or intraperitoneally) to a subject an effective amount of a labeled antibody or fragment thereof against a cancer antigen, to a subject that has been treated with an ARCM or is being treated with the ARCM; b) waiting for a time interval following the administering for permitting the labeled antibody to preferentially concentrate at sites in the subject where target is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of target. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography, X-radiography, nuclear magnetic resonance imaging (NMR), CAT-scans or electron spin resonance imaging (ESR).

### Pharmaceutical Compositions

The compositions described herein can be incorporated into a variety of formulations for administration. More particularly, the compositions can be formulated into pharmaceutical compositions by combination with appropriate, pharmaceutically acceptable carriers or diluents, and can be formulated into preparations in semi-solid, liquid or gaseous forms; such as capsules, powders, granules, gels, slurries, ointments, solutions, suppositories, injections, inhalants and aerosols. As such, administration of the compositions can be achieved in various ways, including oral, buccal, rectal, parenteral, intraperitoneal, intradermal, transdermal, intratracheal administration. Moreover, the compositions can be administered in a local rather than systemic manner, in a depot or sustained release formulation.

In addition, the compositions can be formulated with common excipients, diluents or carriers, and compressed into tablets, or formulated as elixirs or solutions for convenient oral administration, or administered by the intramuscular or intravenous routes. The compositions can be administered transdermally, and can be formulated as sustained release dosage forms and the like. Compositions can be administered alone, in combination with each other, or they can be used in combination with other known compounds (discussed herein).

Suitable formulations for use in the present invention are found in Remington's Pharmaceutical Sciences (1985). Moreover, for a review of methods for drug delivery, see, Langer (1990) Science 249:1527-1533. The pharmaceutical compositions described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

For oral administration, the compositions can be formulated by combining with pharmaceutically acceptable carriers that are known in the art. Such carriers enable the compounds to be formulated as pills, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compositions with an excipient and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions or emulsions in oily or aqueous vehicles, and can contain formulator agents such as suspending, stabilizing and/or dispersing agents.

The compositions can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition, the compositions can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Lipid particles (e.g., liposomes) and emulsions are known examples of delivery vehicles or carriers for hydrophobic drugs. Long-circulating, e.g., stealth, liposomes can be employed. Such liposomes are generally described in U.S. Pat. No. 5,013,556. The compounds of the present invention can also be administered by controlled release means and/or delivery devices such as those described in U.S. Pat. Nos. 3,845,770; 3,916,899; 3,536,809; 3,598,123; and 4,008,719.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. In general, a suitable daily dose of an AHCM agent and/or a cancer therapeutic can be that amount of the compound which is the lowest dose effective to produce a therapeutic effect. Such an effective dose can generally depend upon the factors described above.

The subject receiving this treatment is any animal in need, including primates, in particular humans, equines, cattle, swine, sheep, poultry, dogs, cats, mice and rats.

The compounds can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

Since the AHCM agents are administered in combination with other treatments (such as additional chemotherapeutics, radiation or surgery) the doses of each agent or therapy can be lower than the corresponding dose for single-agent therapy. The determination of the mode of administration and the correct dosage is well within the knowledge of the skilled clinician.

In certain embodiments, the AHCM (alone or in combination with the microenvironment modulator and/or cancer therapy) is formulated for oral, subcutaneous, intravenous or intraperitoneal administration. In one embodiment, the AHCM (alone or in combination with the microenvironment modulator and/or cancer therapy) is formulated for oral administration (e.g., an oral tablet or pill).

As described in the Examples herein, substantially continuous administration of an AHCM (e.g., via a subcutaneous pump) causes a greater reduction in collagen content and/or tumor size than single or pulsatile administration of the AHCM. Thus, it may be desirable to formulate and/or administered the AHCM (alone or in combination with the microenvironment modulator and/or cancer therapy) substantially continuously.

In one embodiment, the AHCM (alone or in combination) is administered substantially continuously over a pre-determined period of, or at least 15, 30, 45 minutes; a period of, or at least, 1, 5, 10, 24 hours; a period of, or at least, 2, 5, 10, 14 days; a period of, or at least, 3, 4, 5, 6, 7, 8 weeks; a period of, or at least 2, 3, 4, 5, 6, 7, 8, 9, 10, 11 months; a period of, or at least, 1, 2, 3, 4, 5 years, or longer. The delivery method can be optimized such that an AHCM dose as described herein (alone or in combination) is administered and/or maintained in the subject for a pre-determined period (e.g., a period as described herein).

The ARCM (alone or in combination with the microenvironment modulator and/or cancer therapy) is in a controlled- or extended release formulation, dosage form, or device. Exemplary formulations and devices for controlled or extended release are known in the art. For example, formulations containing polymer matrices, such as hydroxypropylmethyl cellulose, gels, osmotic systems, liposomes and combination thereof can be used to provide the desired release kinetics.

In one embodiment, the AHCM is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

### Nanoparticles

ARCM agents described herein, the anti-cancer agents (e.g., low molecular weight, mid-molecular weight anti-cancer agents described herein), or both, can be packaged in nanoparticles.

Typically nanoparticles are from 10, 15, 20, 25, 30, 35, 45, 50, 75, 100, 150 or 200 nm or 200-1,000, e.g., 10, 15, 20, 25, 30, 35, 45, 50, 75, 100, 150, or 200, or or 30 or 50-400 nm in diameter. Smaller particles tend to be cleared more rapidly form the system. Drugs can be intrapped within or coupled, e.g., covalent coupled, or otherwise adhered, to nanoparticles.

Lipid- or oil-based nanoparticles, such as liposomes and solid lipid nanoparticles and can be used to can be used to deliver agents described herein. DOXIL® is an example of a liposomic nanoparticle. Solid lipid nanoparticles for the delivery on anti-cancer agents are descripbed in Serpe et al. (2004) Eur. J. Pharm. Bioparm. 58:673-680 and Lu et al. (20060 Eur. J. Pharm. Sci. 28: 86-95. Polymer-based nanoparticles, e.g., PLGA-based nanoparticles can be used to deliver agents described herein. These tend to rely on biodegradable backbone with the theraeutic agent intercalated (with or without covalent linkage to the polymer) in a matrix of polymer. PLGA is a widely used in polymeric nanoparticles, see Hu et al. (2009) J. Control. Release 134:55-61; Cheng et al. (2007) Biomaterials 28:869-876, and Chan et al. (2009) Biomaterials 30:1627-1634. PEGylated PLGA-based nanoparticles can also be used to deliver anti-cancer agents, see, e.g., Danhhier et al., (2009) J. Control. Release 133:11-17, Gryparis et al (2007) Eur. J. Pharm. Biopharm. 67:1-8. Metal-based, e.g., gold-based nanoparticles can also be used to deliver anti-cancer agents. Protein-based, e.g., albumin-based nanoparticles can be used to deliver agents described herein. E.g., an agent can be bound to nanoparticles of human albumin. An exemplary anti-cancer agent/protein nanoparticle is Abraxane®, in which paclitaxel is pund to nanparticles of albumin.

Nanoparticles can employ active targeting, passive targeting or both. Active targeting can rely on inclusion of a ligand that binds with a target at or near a preselected site, e.g., a solid tumor. Passive targeting nanoparticles can diffuse and accumulate at sites of interest, e.g., sites characterized by excessively leaky microvasculature, e.g., as seen in tumors and sites of inflammation.

A broad range of nanoparticles are known in the art. Exemplary approaches include those described in WO2010/005726, WO2010/005723 WO2010/005721, WO2010/121949, WO2010/0075072, WO2010/068866, WO2010/005740, WO2006/014626, 7,820,788, 7,780,984, the contents of which are incorporated herein in reference by their entirety.

### EXAMPLES

The invention now being generally described, it will be more readily understood by reference to the following examples, which are included merely for purposes of illustration of certain aspects and embodiments of the present invention, and are not intended to limit the invention.

In Examples 1-6 below, the inventors assessed if an ARCM agent, for example, losartan—a clinically approved angiotensin II receptor antagonist generally for treatment of hypertension—can enhance the penetration and efficacy of nanomedicine, e.g., via an anti-fibrotic effect. Although nanotherapeutics have offered new hope for cancer treatment, their clinical efficacy is modest (Jain R K, et al. (2010) Nat Rev Clin Oncol 7:653-664; Davis M E, et al. (2008) Nat Rev Drug Discov 7:771-782; Peer D, et al. (2007) Nat Nanotechnol 2:751-760; and Torchilin V P (2005) Nat Rev Drug Discov 4:145-160). Without wishing to be bound by theory, the dense collagen network in tumors can generally reduce the penetration and efficacy of nanotherapeutics. This is partly because their penetration is hindered specially in fibrotic tumors where the small interfibrillar spacing in the interstitium retards the movement of particles larger than 10 nanometers (Netti P A, et al. (2000) Cancer Res 60:2497-2503; Pluen A, et al. (2001) Proc Natl Acad Sci USA 98:4628-4633; Ramanujan S, et al. (2002) Biophys J 83:1650-1660 and Brown E, et al. (2003) Nat Med 9:796-800). Pegylated liposomal doxorubicin (DOXIL®), approved by the FDA, and oncolytic viruses, currently in multiple clinical trials, represent two nanotherapeutics whose size (˜100 nm) hinders their intratumoral distribution and therapeutic effectiveness (Nemunaitis J, et al. (2001) J Clin Oncol 19:289-298). Matrix modifiers like bacterial collagenase, relaxin, and matrix metalloproteinase-1 and -8 have been used to modify the collagen or proteoglycan network in tumors and have improved the efficacy of intratumorally (i.t.) injected oncolytic viruses (Brown E, et al. (2003) Nat Med 9:796-800; McKee T D, et al. (2006) Cancer Res 66:2509-2513; Mok W, et al. (2007) Cancer Res 67:10664-10668; Ganesh S, et al. (2007) Cancer Res 67:4399-4407; and Kim J-H, et al. (2006) J Natl Cancer Inst 98:1482-1493). In addition, relaxin can improve transport through the tumor matrix, but may not facilitate the delivery of low molecular weight agents (U.S. Pat. No. 6,719,977). However, these agents may produce normal tissue toxicity (e.g., bacterial collagenase) or increase the risk of tumor progression (e.g., relaxin, matrix metalloproteinases).

Losartan (Johnston C I (1995) Lancet 346:1403-1407)—approved to control hypertension in patients—does not have many of these safety risks. Furthermore, in addition to its antihypertensive properties, losartan is also an antifibrotic agent that has been shown to reduce the incidence of cardiac and renal fibrosis (Habashi J P, et al. (2006) Science 312:117-121; and. Cohn R D, et al. (2007) Nat Med 13:204-210). The antifibrotic effects of losartan are caused, in part, by the suppression of active transforming growth factor-131 (TGF-β1) levels via an angiotensin II type I receptor (AGTR1) mediated down-regulation of TGF-β1 activators like thrombospondin-1 (TSP-1) (Habashi J P, et al. (2006) Science 312:117-121; Cohn R D, et al. (2007) Nat Med 13:204-210; Lavoie P, et al. (2005) J Hypertens 23:1895-1903; Chamberlain J S (2007) Nat Med 13:125-126; and Dietz H C (2010) J Clin Invest 120:403-407).

As demonstrated below, an AHCM agent, e.g., losartan inhibited collagen I production by carcinoma associated fibroblasts (CAFs) isolated from breast cancer biopsies. Additionally, an AHCM agent, e.g., losartan, led to a dose-dependent reduction in stromal collagen in desmoplastic models of human breast, pancreatic and skin tumors in mice. Furthermore, an AHCM agent, e.g., losartan improved the distribution and therapeutic efficacy of intratumorally injected oncolytic herpes simplex viruses (HSV). Further, an AHCM agent, e.g., losartan also enhanced the efficacy of intravenously injected pegylated liposomal doxorubicin (DOXIL®). Accordingly, administration of an AHCM agent, e.g., losartan, in combination with a cancer therapeutic (e.g., a cancer nanotherapeutic) can enhance the efficacy of nanotherapeutics in patients with desmoplastic tumors.

Using a dose that has minimal effects on mean arterial blood pressure (MABP), the inventors have shown below that an AHCM agent, e.g., losartan reduces collagen I levels in four tumor models—a spontaneous mouse mammary carcinoma (FVB MMTV PyVT), an orthotopic pancreatic adenocarcinoma (L3.6 μl), and subcutaneously implanted fibrosarcoma (HSTS26T) and melanoma (Mu89). Further, the inventors have shown below that an AHCM agent, e.g., Losartan, can also improve the intratumoral penetration of nanoparticles injected intratumorally (i.t.) or intravenously (i.v.).

Additionally, the inventors assessed how an AHCM agent, e.g., losartan, can affect the distribution and efficacy of oncolytic HSV administered i.t.—a widely used method of administration in patients for gene therapy (Hu J C, et al. (2006) Clin Cancer Res 12:6737-6747; Senzer N N, et al. (2009) J Clin Oncol 27:5763-5771; Breitbach C J, et al. (2010) Cytokine Growth Factor Rev 21:85-89)—and the efficacy of i.v.-administered DOXIL®. As shown below, an AHCM agent, e.g., Losartan, improved the efficacy of both i.t.-injected oncolytic HSV and i.v.-administered, DOXIL®. The results from the intratumoral (i.t.) experiments indicate that an AHCM agent, e.g., losartan, can enhance nanoparticle penetration in the interstitial space by improving interstitial transport. Additionally, the findings from the intravenous (i.v.) studies indicate that an AHCM agent (e.g., losartan) can improve the efficacy of systemically administered nanotherapeutics to fibrotic solid tumors, even highly fibrotic solid tumors, such as pancreatic adenocarcinomas. Accordingly, an AHCM agent, e.g., losartan, an FDA approved antihypertensive drug, can be used to improve the efficacy of various nanotherapeutics in multiple tumor types.

### Example 1

### Losartan Inhibits Collagen I Synthesis by Carcinoma Associated Fibroblasts (CAFs)

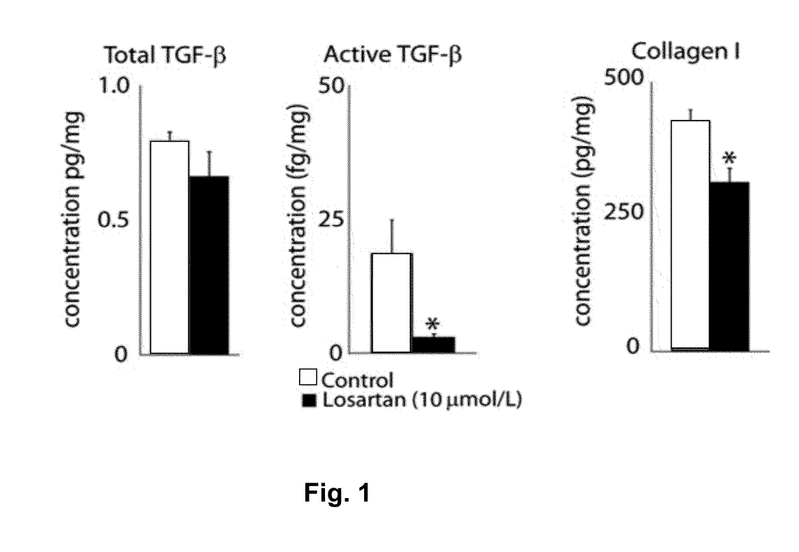

The effect of losartan on the expression and activation of TGF-β1, and collagen I production by mammary CAFs was examined (FIG. 1). Losartan reduces TGF-β1 activation and collagen I production in carcinoma associated fibroblasts in Vitro. Cells were treated with 10 μmol/L of losartan for 24 hrs. Losartan reduced by 90% the active-TGF-β1 levels while total TGF-β1 levels were unaffected. There was a corresponding 27% decrease in collagen I levels. The reduction in active-TGF-β1 and collagen I was statistically significant (student t-test p<0.05). Since collagen in tumors is mostly produced by CAFs, the effect of losartan in the collagen content in tumors was examined.

### Example 2

### Losartan Decreases Collagen I in Tumors in a Dose-Dependent Manner

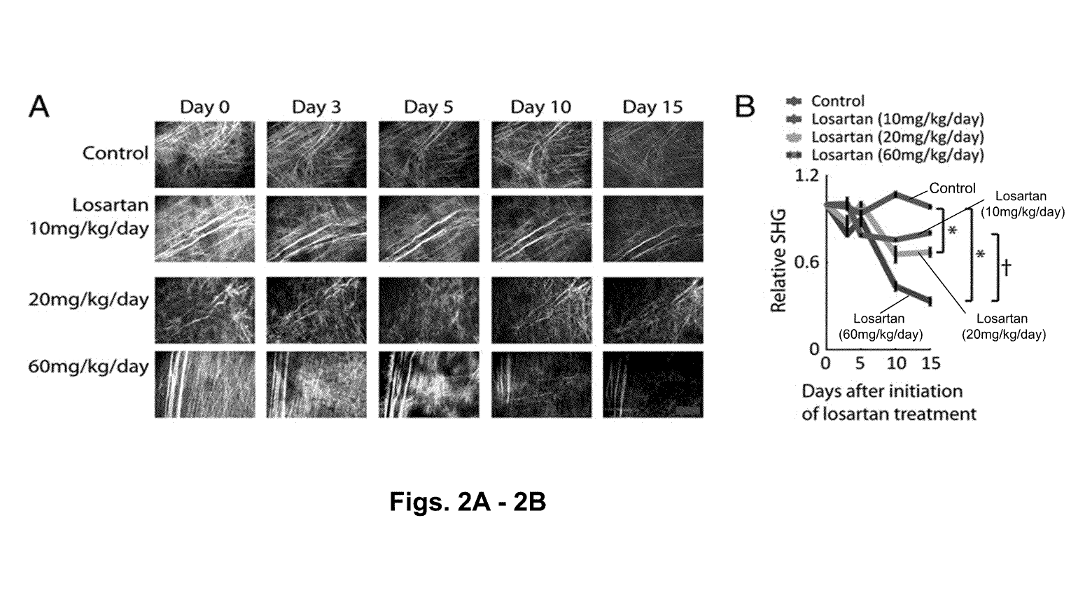

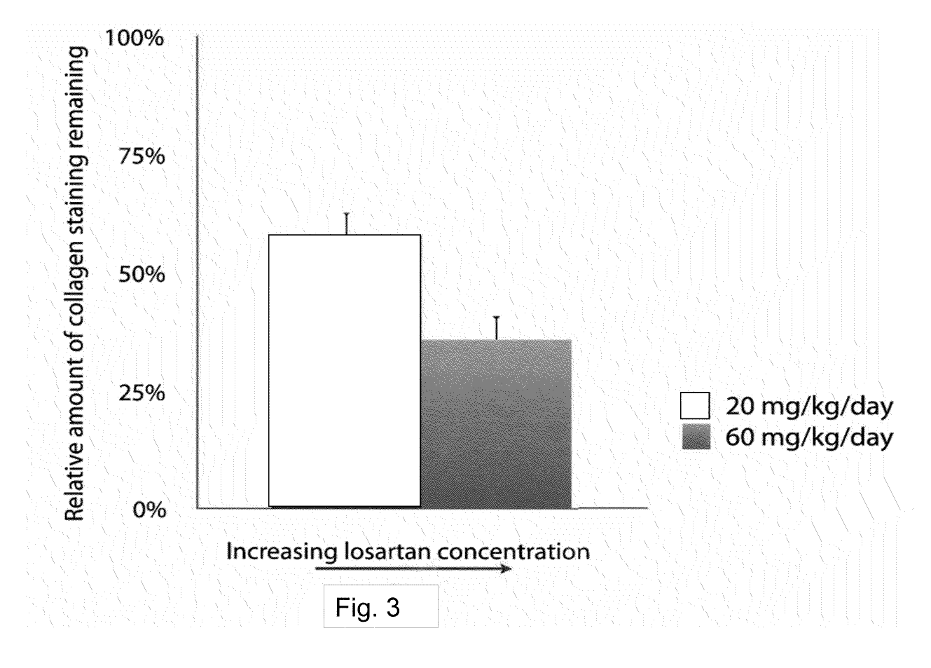

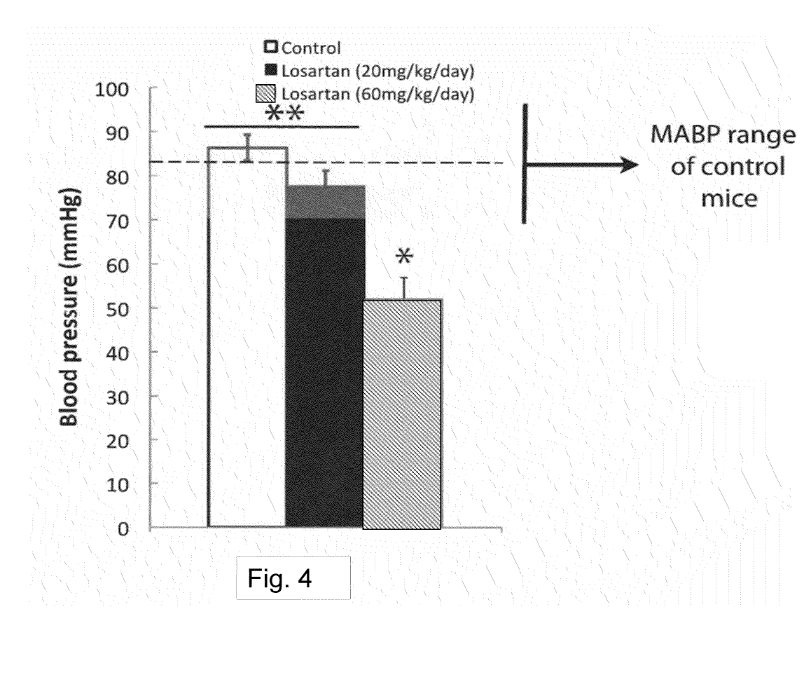

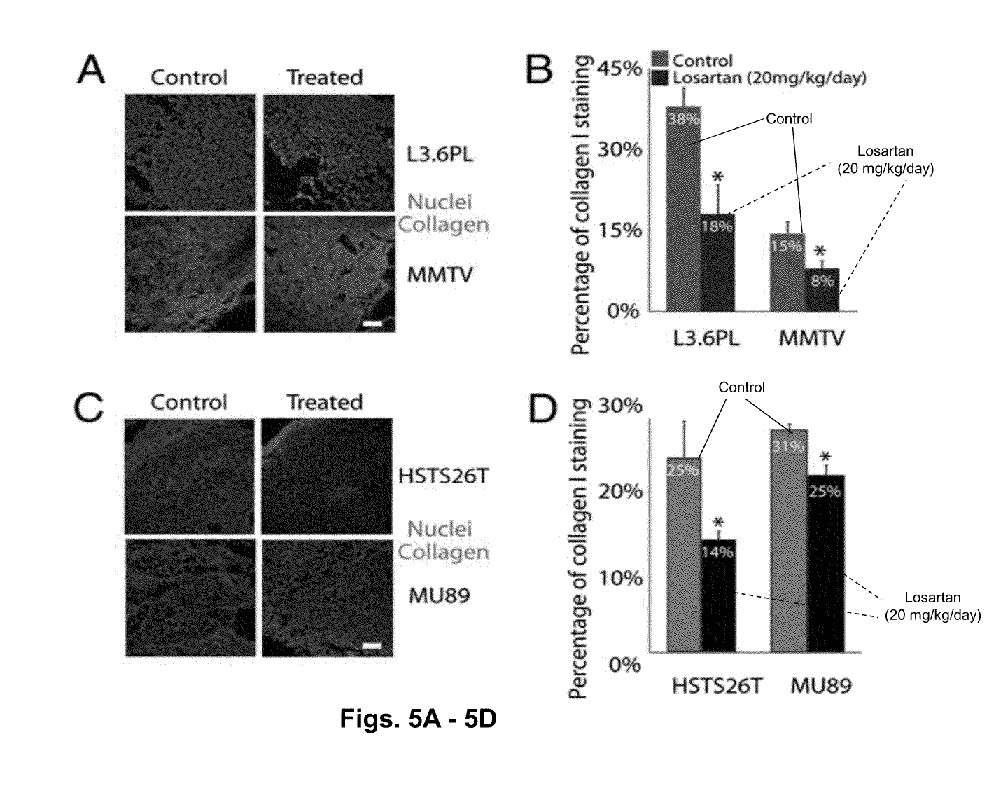

To determine the dose-response of losartan on intratumoral collagen levels, 10, 20, and 60 mg/kg/day of losartan were injected intraperitoneally (i.p.), and performed second harmonic generation (sHG) imaging of fibrillar collagen in HSTS26T tumors in dorsal skin fold chambers (FIGS. 2A-2B) and collagen I immunostaining of tumor sections (FIGS. 3A-3D). While the SHG signal intensity can include signals contributed from collagen I and other fibril-forming collagens (e.g., collagen III or V), collagen I is generally the predominant collagen type in most soft tissues (Gelse K, et al. (2003) Adv Drug Deliv Rev 55:1531-1546), and thus contributes as the main source of the SHG signal. Additionally, in human pancreatic tumors collagen I is the main fibrillar collagen with significantly lower levels of collagen V (Mollenhauer J, et al. (1987) Pancreas 2:14-24). Losartan doses of 20 and 60 mg/kg/day significantly reduced the intratumoral SHG signal intensity, whereas the lowest dose of 10 mg/kg/day did not have a significant effect on the SHG signal intensity (FIGS. 2A and 2B). The injection of losartan at 60 and 20 mg/kg/day also significantly reduced the collagen I immunostaining in HSTS26T tumors by 65% and 42%, respectively (FIG. 3). Treatment with the 60 mg/kg/day dose led to the highest reduction in collagen I, with a reduction in the mean arterial blood pressure (MABP) by 35 mm Hg (p<0.04; FIG. 4). In the following Examples, 20 mg/kg/day dose was used (but it by no means limits the use of other doses in the methods described herein). After 2 weeks of losartan treatment, the 20 mg/kg/day dose reduced the MABP by 10 mm Hg (FIG. 4), thus maintaining the MABP within the normal range (70-95 mmHg) for SCID mice (Kristjansen P E, et al. (1993) Cancer Res 53:4764-4766). It also had no detectable effect on mouse weight (average of 26±1 g treated vs. 26±1 g control). The 20 mg/kg/day dose decreased collagen I immunostaining in four tumor types—FVB MMTV PyVT, L3.6 μl, HSTS26T, and Mu89-by 47% (p<0.05), 50% (p<0.03), 44% (p<0.04), and 20% (p<0.02), respectively (FIGS. 5A-5D).

### Example 3

### Losartan Decreases TSP-1 Expression in Tumors

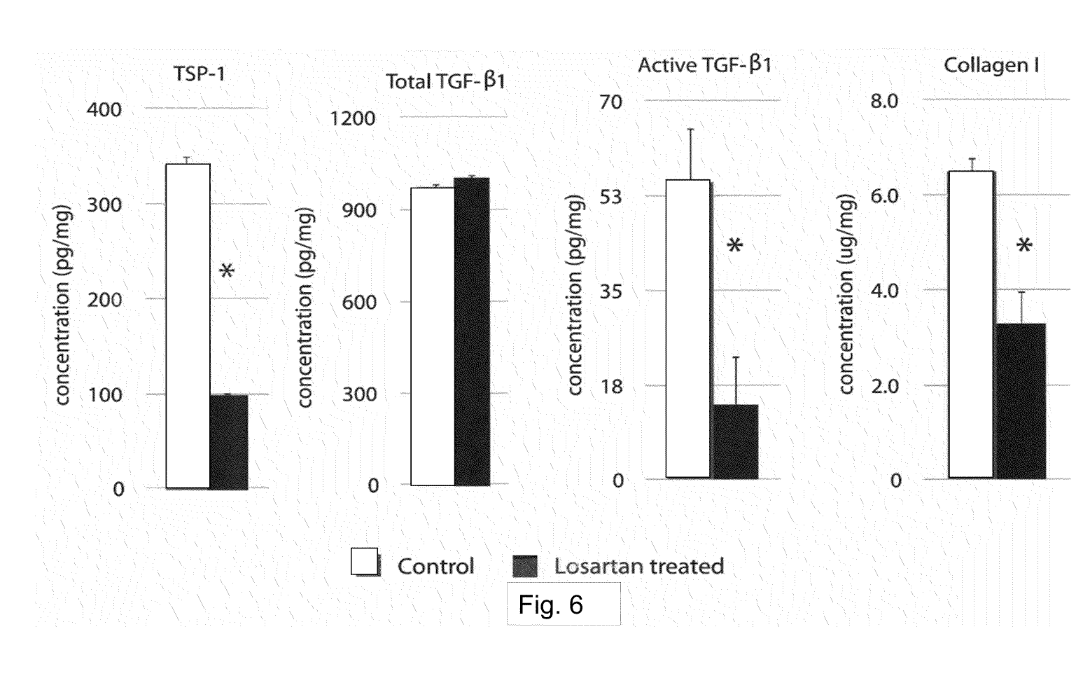

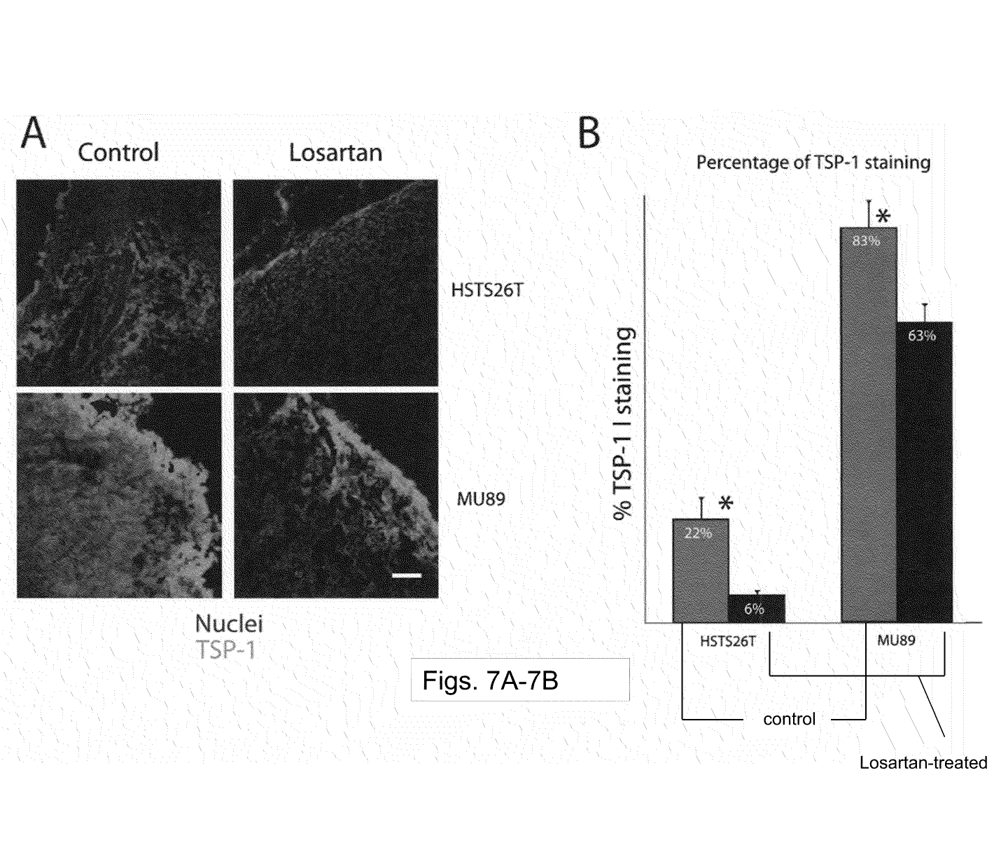

TSP-1 is a key regulator of TGF-β1 activation and losartan has been reported to reduce TSP-1 expression and TGF-β1 activation in mouse models of Marfan's syndrome and muscular dystrophy (Dietz H C (2010) J Clin Invest 120:403-407). As shown herein, the measurement of protein levels in homogenized HSTS26T tumors showed that losartan did not affect total TGF-β1 levels but significantly reduced TSP-1, active TGF-β1, and collagen I levels (FIG. 6). Losartan also decreased the TSP-1 immunostaining in HSTS26T (73% p<0.04) and Mu89 (24% p<0.03) (FIGS. 7A-7B). In both Mu89 and HSTS26T tumors the immunostaining patterns for TSP-1 (FIGS. 7A-7B) and collagen I (FIGS. 5C-5D) were closely matched. The inventors detected high levels of TSP-1 and collagen I in the tumor margin, while losartan induced obvious reductions in TSP-1 and collagen I levels in the tumor center (FIGS. 5C, 7A). These data indicate that the reduction in collagen I levels can result in part from the decreased activation of TGF-β1 due to the losartan-induced reduction in TSP-1 expression.

### Example 4

### Losartan Improves the Intratumor Distribution of Nanoparticles and nanotherapeutics

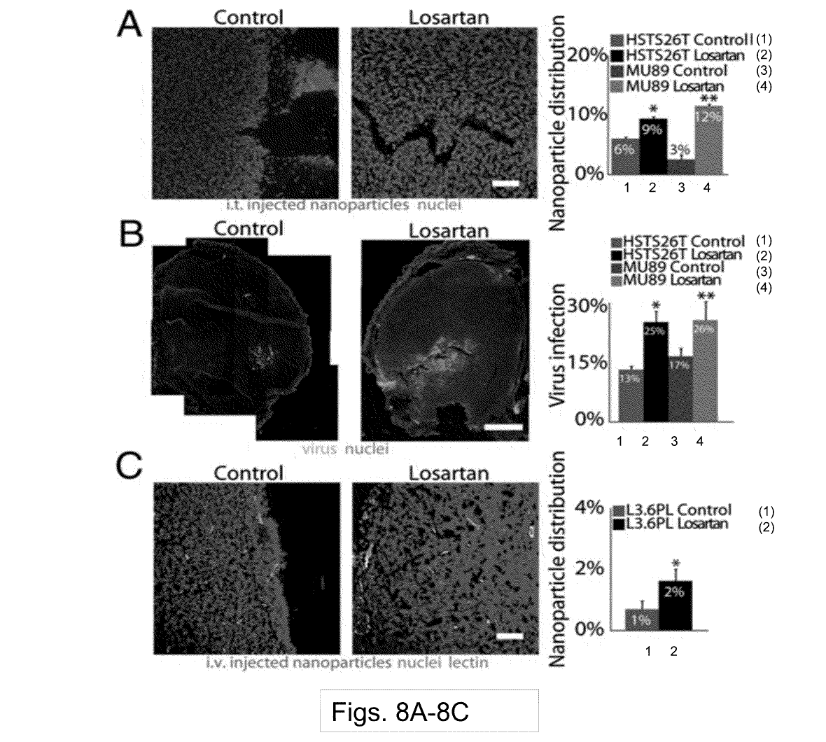

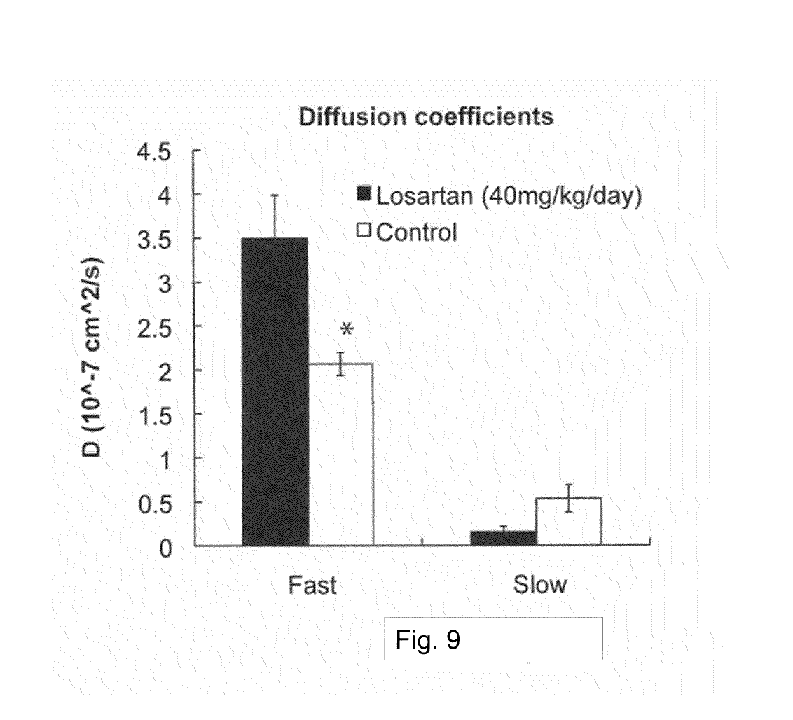

Based on the previous studies on the tumor interstitial matrix (Pluen A, et al. (2001) Proc Natl Acad Sci USA 98:4628-4633 and Brown E, et al. (2003) Nat Med 9:796-800), the inventors then sought to determine if a decrease in collagen content by Losartan can improve the intratumoral distribution of nanoparticles. The inventors therefore measured the intratumoral distribution of fluorescent polystyrene nanoparticles (100 nm diameter) in three different tumor types—HSTS26T, Mu89, and L3.6 pl—after an i.t. or i.v. injection. In mice injected i.t. with nanoparticles, losartan improved nanoparticle accumulation and penetration in the tumor center (FIG. 8A; HSTS26T p<0.001, Mu89 p<0.001). Conversely, there was little or no nanoparticle accumulation in the center of control tumors. Most of the injected nanoparticles in control tumors were found in the tumor margin and around the needle insertion point (FIG. 8A). The inventors also determined the effects of losartan on the intratumoral distribution of oncolytic HSV. In both HSTS26T and Mu89, losartan significantly increased the intratumoral spread of HSV injected intratumorally (FIG. 8B). While these findings show that losartan increases the distribution of large nanoparticles, the inventors also determined that in HSTS26T, losartan increased interstitial diffusion of IgG (FIG. 9) and the mean interstitial matrix pore radius—from 9.91±0.43 nm to 11.78±0.41 nm, calculated based on IgG diffusion data (Nugent U, et al. (1984) Cancer Res 44:238-244).

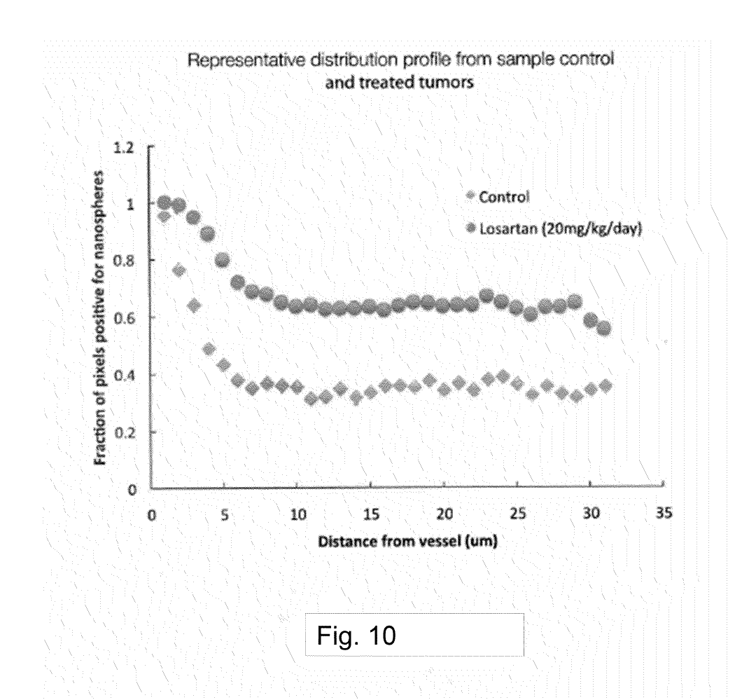

The inventors then assessed the effect of losartan on blood vessel perfusion and the intratumoral distribution of i.v. injected nanoparticles in mice with orthotopic pancreatic tumors (L3.6 pl). The intratumoral accumulation and penetration of beads away from blood vessels was significantly higher in losartan-treated tumors (FIG. 8C and FIG. 10). These results indicate that losartan improves the transport and distribution of both i.t. and/or i.v. injected nanoparticles.

### Example 5

### Losartan Improves the Efficacy of DOXIL® and Oncolvtic HSV

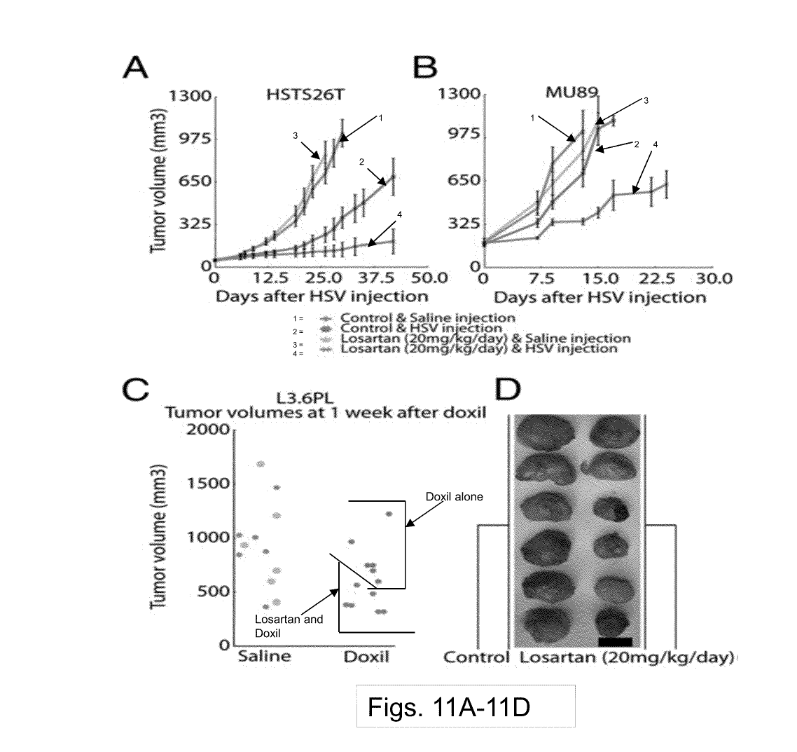

The inventors then determined if losartan could improve the efficacy of i.t. injected oncolytic HSV and iv. injected DOXIL®. The effect of losartan combined with the i.t. injection of HSV was determined in HSTS26T and Mu89 tumors. The administration of losartan alone did not affect the tumor growth rate (FIGS. 11A and 11B). However, when animals were treated with losartan for two weeks before i.t. injection of HSV, losartan significantly delayed the growth in both Mu89 and HSTS26T tumors (FIGS. 11A and 11B). The volume of HSTS26T tumors remained stable for up to 9 weeks in 50% of mice treated with losartan and HSV. For the Mu89 tumors, mice treated with losartan and HSV had a delay in tumor growth. However, the growth delay in Mu89 tumors was only transient, 4 weeks after the virus injection all the tumors were 3-fold larger than the starting treatment size.

To determine if losartan could increase the efficacy of a nanotherapeutic injected i.v., mice with orthotopic pancreatic tumors (L3.6 μl) were treated with DOXIL® and losartan. Four weeks after tumor implantation and two weeks after initiation of losartan treatment (20 mg/kg/day), the inventors treated mice with a sub-anti-tumor dose (i.e., a dose that is not effective for treatment of cancer, e.g., a dose that is not effective to inhibit or prevent tumor growth and/or progression) of DOXIL® (4 mg/kg, i.v.). After 7 days, losartan or DOXIL alone did not affect the mean tumor weight (FIG. 11C). However, in mice treated with losartan and DOXIL® the tumors were significantly smaller (p<0.001) than in mice that received DOXIL® alone (FIGS. 11C and 11D).

### Example 6

### The Pattern of Collagen Distribution Regulates the Effectiveness of losartan

To investigate the differences in response between HSTS26T and Mu89 to the losartan-HSV combination therapy, the inventors determined the HSV infection and necrosis patterns 21 days after the i.t. injection of HSV. FIGS. 12A and 12B show striking differences between the collagen structure in Mu89 (FIG. 12A) and HSTS26T (FIG. 12B) tumors, respectively. Without wishing to be bound by theory, these differences in the collagen structure altered the virus propagation in these tumor types. In Mu89 tumors the collagen fiber network was well organized and formed finger-like projections into the tumor (FIGS. 12A and 13A). These projections divided the tumor into distinct compartments, which could not be crossed by HSV particles, thus the virus infection and resulting necrosis was restricted to the infected compartments (FIG. 14A). Losartan treatment disrupted the collagen projections to some extent but did not completely eliminate them (FIG. 12A). As a result, there was some crossover of virus particles between compartments in losartan-treated Mu89 tumors. By varying Losartan and/or HSV concentration the amount or extent of collagen projections to be disrupted (e.g., partial or complete disruption) can be modulated, and thus in turn affect the distribution of the HSV particles within the tumor. In contrast, in HSTS26T tumors the dense collagen network was more diffuse, less fibrillar and less compartmentalized (FIGS. 12B and 13B). The dense collagen network seemed to slow down virus propagation but did not completely impede it, resulting in increased virus propagation and a more diffuse pattern of necrosis in this tumor (FIG. 14A).

### Discussion

The renin-angiotensin-aldosterone system (RAAS) has been reported to play a role in the regulation and production of extracellular matrix components (Cook K L, et al. (2010) Cancer Res 70:8319-8328; Rodriguez-Vita J, et al. (2005) Circulation 111:2509-2517; and Wolf G (2006) Kidney Int 70:1914-1919). Angiotensin II has been reported to stimulate collagen production via both TGF-β1 dependent and independent pathways (Yang F, et al. (2009) Hypertension 54:877-884). Losartan and other RAAS inhibitors have reported to reduce the levels of collagen I and III, and basement membrane collagen IV in various experimental models of fibrosis (Toblli J E, et al. (2002) J Urol 168:1550-1555 and Boffa J J, et al. (2003) J Am Soc Nephrol 14:1132-1144), and reverse renal and cardiac fibrosis in hypertensive patients (Lim D S, et al. (2001) Circulation 103:789-791 and Khalil A, et al. (2000) J Urol 164:186-191). Using four different tumor types, the inventors have demonstrated herein for the first time that losartan also inhibits collagen I production in tumors.

Other matrix modifiers like bacterial collagenase, relaxin, and matrix metalloproteinase-1 and -8 have been reported to modify the collagen or proteoglycan network in tumors and have improved the efficacy of oncolytic virus injected intratumorally (Brown E, et al. (2003) Nat Med 9:796-800; McKee T D, et al. (2006) Cancer Res 66:2509-2513; Mok W, et al. (2007) Cancer Res 67:10664-10668; Ganesh S, et al. (2007) Cancer Res 67:4399-4407; and Kim J-H, et al. (2006) J Natl Cancer Inst 98:1482-1493). However, these agents may produce normal tissue toxicity (e.g., bacterial collagenase) or increase the risk of tumor progression (e.g. relaxin, matrix metalloproteinases). In contrast, losartan (Johnston C I (1995) Lancet 346:1403-1407) has limited side effects. Losartan has been reported to reduce the incidence of metastasis in some tumor types (Arafat H A, et al. (2007) J Am Coll Surg 204:996-1005).

As shown in the Examples above, an AHCM agent, e.g., losartan, can reduce collagen content and in turn improve interstitial transport and the intratumoral distribution of nanoparticles and nanotherapeutics. The inventors also discovered that the organization of the collagen fibrillar network can affect nanoparticle distribution. This was striking because of significant differences in the structural organization of fibrillar collagen I between Mu89 and HSTS26T. In Mu89 tumors, thick bundles of fibrillar collagen I surround the tumor margins and form finger-like projections, which subdivide the tumor mass into isolated compartments and confine the viral infection to the injection site/isolated compartments (FIGS. 12A and 13A). In contrast HSTS26T tumors have a mesh-like collagen structure, which hinders the virus spread but does not restrict viral particles to the injection site (FIGS. 12B and 13B). The slower growth rate of HSTS26T than Mu89 tumors could also explain in part the enhanced efficacy of losartan combined with HSV in HSTS26T tumors. Accordingly, not only the collagen content but also the collagen network organization plays an important role in limiting the penetration of large therapeutics in tumors. Depending on the content and/or organization of the collagen network within certain tumors, doses, administration methods and/or frequency of an AHCM agent (e.g., losartan) and/or a cancer therapeutic (e.g., HSV) can be adjusted accordingly.

Pancreatic cancer patients treated with cytotoxic agents have a very high frequency of relapse with a 5 year survival of less than 5% (Li J, et al. (2010) AAPS J 12:223-232). The poor vascular supply and increased fibrotic content of pancreatic tumors most likely play a significant role in limiting the delivery and efficacy of cytotoxics (Olive K P, et al. (2009) Science 324:1457-1461). The inventors show—in a mouse orthotopic model of human pancreatic cancer (L3.6 μl)—that losartan increases both the intratumoral dispersion and extravascular penetration distance of i.v. injected nanoparticles. The increased distribution and extravasation of nanoparticles indicate that losartan can not only improve interstitial transport—as shown with the i.t. injections of nanoparticles and virus—but also transvascular transport. When used alone, losartan did not affect the growth of pancreatic tumors or the weight of treated mice. However, losartan combined with DOXIL® reduced the tumor sizes by 50% compared to DOXIL® treatment alone. These findings indicate that losartan increased the tumor penetration and distribution, and enhanced efficacy of DOXIL® injected i.v. in orthotopic pancreatic carcinomas in mice.

The effects of losartan are not limited to the interstitial space. Modifications to the RAAS system can also inhibit angiogenesis (Fujita M, et al. (2005) Carcinogenesis 26:271-279) or alter tumor blood flow (Jain R, et al. (1984) IEEE Trans Son Ultrason 31:504-526 and Zlotecki R A, et al. (1993) Cancer Res 53:2466-2468). Losartan-blockade of AGTR1 can also reduce the production of VEGF by cancer cells and the expression of VEGFR1 in endothelial cells, and inhibit tumor angiogenesis and growth (Otake A H, et al. (2010) Cancer Chemother Pharmacol 66:79-87 and Noguchi R, et al. (2009) Oncol Rep 22:355-360). As shown herein, losartan did not affect tumor growth or the vascular density in HSTS26T tumors. Losartan can also reduce the proliferation of tumor cells expressing AGTR1 (Rhodes D R, et al. (2009) Proc Natl Acad Sci USA 106:10284-10289). The inventors did not find a decrease in cancer cell proliferation (FIG. 15) or tumor size in the human melanoma Mu89, which express AGTR1 (FIG. 16). The difference between their study and other prior studies might be due to differences in dosage. For example, in prior studies the dose of losartan was up to 15 fold higher than what was used in the inventors' study (Otake All, et al. (2010) Cancer Chemother Pharmacol 66:79-87). The inventors have shown herein that a low dose of losartan that is ineffective for treatment of cancer by itself alone, can be used to improve the efficacy of a cancer therapy or an anti-cancer agent (even at a sub-therapuetic level) for treatment of cancer. Further, the low dose of losartan can allow for a more clinically translatable protocol and avoid hypotensive complications.

Patients receiving RAAS antagonists have reduced incidence of breast and lung cancer (Lever A F, et al. (1998) Lancet 352:179-184). Different mechanisms have been reported to discuss the anti-tumor properties of RAAS antagonists when used at high concentrations (Ager E I, et al. (2008) Carcinogenesis 29:1675-1684; Lindberg H, et al. (2004) Acta Oncol 43:142-152; Miyajima A, et al. (2002) Cancer Res 62:4176-4179 and Rosenthal T, et al. (2009) J Hum Hypertens 23:623-635). AGTR1 signaling has been reported to increase the proliferation of stromal and tumor cells, and the transcription of inflammatory cytokines and chemokines that promote cancer cell migration and dissemination (Deshayes F, Nahmias C (2005) Endocrinol Metab 16:293-299). The reduction in active TGF-β1 levels by RAAS antagonists administered at high concentrations have been reported to reduce metastasis (Jakowlew S B (2006) Cancer Metastasis Rev 25:435-457). Accordingly, in addition to improving the delivery of antitumor agents, losartan can also inhibit tumor progression and metastasis. By way of example only, losartan administered at a low dose (e.g., a dose not effective to reduce or prevent metastasis if administered alone) with an anti-metatstic agent (e.g., at a dose less than what is typically administered by itself for treatment and/or prevention of metastasis) can be used to inhibit tumor progression and metastasis.

In order to use losartan as an adjunct in the treatment of cancer patients it is important to consider dosing and treatment schedules along with potential side effects. Results from the dose and time dependent studies presented herein indicate a minimum of two weeks of losartan administration prior to anti-tumor treatment. To obtain maximum effects in patients, it might be prudent to initiate losartan treatment two weeks prior to and continue it during the entire antitumor treatment schedule. Since long-term losartan therapy in hypertensive patients has been shown to have limited and manageable side effects and many antitumor agents (e.g., anti-VEGF drugs) have been shown to increase blood pressure (Ager E I, et al. (2008) Carcinogenesis 29:1675-1684), extended losartan co-therapy can be beneficial to cancer patients. In some embodiments, patients can be treated with a dose of 2 mg/kg/day losartan, which is generally used for the treatment of patients with Marfan's syndrome (Brooke B S, et al. (2008) N Engl J Med 358:2787-2795).

Although losartan and ARBs have limited side effects, losartan therapy is not recommended for patients with known renal disease. Losartan can induce renal insufficiency in patients with renal microvascular or macrovascular disease, or congestive heart failure (Sica D A, et al. (2005) Clin Pharmacokinet 44:797-814). Hyperkalemia can also occur in patients with poor renal function or patients who are concomitantly receiving potassium supplements or potassium sparing diuretics. Finally, angioedema caused by high levels of circulating angiotensin II can occur in patients treated with losartan (Sica D A, et al. (2005) Clin Pharmacokinet 44:797-814).

Tumor drug resistance is generally believed to occur at many levels including increased drug efflux, drug inactivation, evasion from apoptosis, and alterations in target pathways (Longley D B, et al. (2005) J Pathol 205:275-292). Since losartan is not an antitumor agent, tumor resistance to losartan therapy after extended treatment can result from other mechanisms. Given that TGF-β1 activation is induced by different agents like MMPs and integrins in addition to TSP-1, tumor resistance to losartan could result from changes in TGF-β1 activation and signaling. However, long-term losartan therapy after myocardial infarction has been reported as not being associated with a reduction in antifibrotic properties (Schieffer B, et al. (1994) Circulation 89:2273-2282).

As shown in Examples 1-6, the inventors show that losartan reduces the stromal collagen content in tumors and improves the penetration and therapeutic efficacy of nanoparticles (DOXIL®, HSV) delivered both i.t. and i.v. Losartan also exhibits vasoactive and anti-metastatic properties that could increase its clinical application. Furthermore, since losartan is already approved for clinical use, it represents a safe and effective adjunct for improving the efficacy of nanotherapeutics in cancer patients.

### Exemplary Experimental Protocols for Examples 1-6

### Exemplary Materials and Methods

A more detailed description of techniques is presented in the Additional Materials and Methods section below.

Briefly, CAFs isolated from human breast cancer biopsies were treated with losartan for 24 hrs prior to measurements of collagen and cytokine levels. Protein assays were done with commercial ELISA kits. All animal experiments were done with approval of the Institutional Animal Care and Use Committee. Losartan was administered i.p. at concentrations of 10, 20 or 60 mg/kg/day for up to 2 weeks. Mice were treated with HSV (i.t.) and DOXIL® (i.v. via tail vein) after 2 weeks of losartan treatment. Excised tumors were either snap frozen for biochemical analyses or fixed in paraformaldehyde, and embedded in paraffin or optimum cutting temperature compound (OCT) for immunohistochemistry.

### Additional Materials and Methods:

### Cell Culture

CAFs were isolated from human breast cancer biopsies using an art-recognized protocol, e.g., the protocol described in Orimo A, et al. (2005) Cell 121:335-348. CAFs were plated in 24 well plates at a concentration of 500K cells/well. Cells were allowed 24 hrs to adhere to the plates before the addition of losartan at 10 μmol/l for 24 hrs (Schuttert J B, et al. (2003) Pflugers Arch 446:387-393). Treatment was done in low serum to reduce background collagen levels. Conditioned medium was collected at the end of the 24-hr treatment period and analyzed for collagen levels.

### Protein Assays

Collagen I measurements were done with a type I C-terminal collagen propetide Enzyme Linked Immunosorbent Assay (ELISA) kit (Quidel, San Diego, Calif.) and the Sircol soluble collagen assay (Biocolor Ltd., United Kingdom). TGF-β1 assays were performed with a human TGF-β1 ELISA kit (R&D Systems, Minneapolis, Minn.). The assay only measures the free-form of mature TGF-β1. To measure total levels of TGF-β1 the latent form of TGF-β1 was activated with 1N HCl. TSP-1 assays were performed with a human TSP-1 ELISA kit (R&D Systems, Minneapolis, Minn.).

### Mice and Tumor Models

All experiments were done with approval of the Institutional Animal Care and Use Committee. Human soft tissue sarcoma (HSTS26T) and human melanoma (Mu89) tumors were grown subcutaneously in the legs and dorsal skin fold chamber of severe combined immunodeficient (SCID) mice (Leunig M, et al. (1992) Cancer Res 52:6553-6560). Human pancreatic adenocarcinoma cells (L3.6PL) were grown orthotopically in the pancreas of SCID mice. L3.6PL tumors were induced with a sub-capsular injection of one million cells in the tail of the pancreas. Tumor sizes were monitored in spontaneous FVB/N-Tg (MMTV-PyVT) 634MU1/J mice and tumors selected for treatment when they reached a size of 4 to 6 mm in diameter (Guy C T et al. (1992) Mol Cell Biol 12:954-961).

### Losartan Preparation and Treatment

Cozaar (losartan potassium) tablets were ground using a mortar and pestle. The powder was then dissolved in water to obtain a concentration of 2.5 mg/ml. The solution was then filtered and stored in a sterile container. Losartan was administered by daily i.p. injections at a concentration of 10, 20 or 60 mg/kg/day for up to 2 weeks (Melo L G, et al. (1999) Am J Physiol 277:R624-R630).

### Tissue Collection, Embedding and Staining

Tumors for immunostaining analysis and quantification were harvested from mice, fixed in 4% paraformaldehyde, and embedded in paraffin or optimum cutting temperature compound (OCT) (Sakura Finetek Torrance, Calif.). OCT embedded tumors were soaked in sucrose solution for 24 hrs prior to embedding and freezing.

Collagen I and TSP-1 Immunostaining Staining in Frozen Sections

Frozen sections were cut into 10 μm sections for immunohistochemistry and imaging. Collagen I was detected using the LF-67 antibody (1:100 dilution) with a previously described protocol (Znati C A, et al. (2003) Clin Cancer Res 9:5508-5513). TSP-1 was detected with a goat anti-human antibody (1:50 dilution), which cross-reacts with mice (sc-12312, Santa Cruz Biotechnology Inc., Santa Cruz, Calif.). For collagen and TSP-1 analysis, images at 20× magnification were taken randomly from each slide. The collagen and TSP-1 content was determined by measuring the number of pixels above a threshold value that was set based on the average intensity value of pixels from all slides under analysis. The background-signal intensity for both collagen I and thrombospondin-1 immunostaining was low and uniform. The inventors confirmed that the average signal intensity threshold lead to an accurate representation of the collagen and thrombospondin-1 immunostaining and did not include the background signal.

### Second Harmonic Imaging of Collagen Fibers

Second Harmonic Imaging (SHG) imaging was performed in dorsal chamber tumors with a custom-built multiphoton laser-scanning microscope (Brown E, et al. (2003) Nat Med 9:796-800). Polarized light from a Ti:Sapphire laser (Mai-Tai Broadband: Spectra-Physics, Mountain View, Calif.) was converted to circularly polarized light using a zero order quarter wave plate (Newport Corporation, Irvine, Calif.). An excitation wavelength of 810 nm and detected SHG signals at 405 nm was used. SCID mice bearing HSTS26T tumors in dorsal chambers were either treated with losartan (10, 20 or 60 mg/kg/day) or saline for the duration of the dose response experiment (15 days). Vascular markers were used to locate 4 regions of interest in each mouse and periodically returned to the same region of SHG imaging. SHG images were analyzed with a custom-built Matlab (The MathWorks, Inc., Natick, Mass.) code. The fraction of the region of interest (ROI) that was positive for the SHG signal was normalized to the amount of SHG signal obtained on day 1 of the dose response study (before initiation of losartan or saline treatment).

### Analysis of HSV Infection and Nanoparticle Distribution

Intratumoral Injection:

Nanoparticles and oncolytic HSV were infused with a syringe pump (Harvard Apparatus Standard Pump 22, Holliston, Mass.) at a flow rate of 4 μl/min. The inventors injected 10 μl of HSV (2.5×10⁵ t.u.) expressing the green fluorescent protein (GFP), or 10 μl of fluorescent nanoparticles (diameter of 100 μm; concentration of 1×10¹³ nanoparticles/ml). The injected tumors were resected 30 min after the nanosphere injection and 24 hrs after the HSV infusion. Resected tumors were bisected at an angle perpendicular to the needle track, fixed in paraformaldehyde and frozen in OCT. All tumor sections were obtained perpendicular to the angle of the needle track. The entire tumor section was imaged with a confocal microscope (Olympus BX61WI) at 2× and images were reconstituted as mosaics. The nanosphere distribution and GFP-positive areas (HSV infected cells) corresponds to the fraction of pixels brighter than the background signal.

Intravenous Injection:

A total volume of 10 μl at a concentration of 3.6×10¹³ nanoparticles/ml was injected via the tail vein. Twenty-four hrs later 50 μl of FITC-lectin was injected to identify functional vessels. Five min after the lectin-injection tumors were resected, fixed in paraformaldehyde and embedded in OCT. Tumors were then sectioned before confocal imaging and analysis. The extent of nanosphere distribution was determined by measuring the fraction of pixels brighter than the background signal. Nanosphere penetration was determined by drawing contours around perfused vessels and recording the fraction of pixels positive for nanospheres in each contour. Contours extended out to 30 μm for each perfused vessel. Using a previously described algorithm (Tong R T, et al. (2004) Cancer Res 64:3731-3736), the inventors fit the plot of nanosphere fraction and distance away from the vessel to an exponential and obtained a relative penetration depth of nanospheres from each vessel.

Diffusion Measurements by Fluorescence Recovery after Photobleaching

Mice with HSTS26T tumors implanted in a dorsal skin fold chamber were treated with i.p. injections of losartan (40 mg/kg/day) for 1 week. Fluorescence recovery after photobleaching (FRAP) measurements were done with a custom built multiphoton microscope based on a previously described protocol (Chauhan V P, et al. (2009) Biophys J 97:330-336). IgG labeled fluorescein isothiocyanate (0.5 ml; 2 mg/ml) was injected i.t. and used as the tracer. Diffusion was measured by multiphoton FRAP (MP-FRAP) and spatial Fourier analysis FRAP (SFA-FRAP) about 10 min after the injection. Matrix pore sizes were calculated using the SFA-FRAP data, using the equation

$$\frac{D}{{D}_{0}} = \frac{1 - 2.105 λ + 2.0865 \text{?} - 1.7068 {λ}^{5} + 0.72603 {λ}^{6}}{1 - 0.75857 {λ}^{5}} , \\ \text{?} \text{indicates text missing or illegible when filed}$$

where D is the diffusion coefficient for the probe molecule in the tumor, Do is its diffusion coefficient in water, and A is the ratio of the probe hydrodynamic radius to the pore radius (Nugent L J, et al. (1984) Microvasc Res 28:270-274).

### Analysis of HSV Infection, Necrosis and Collagen Structure

To determine the relationship between virus infection, necrosis and collagen structure 21 days after the HSV injection, consecutive paraffin sections were stained with either a polyclonal HSV-1 antibody (DAKO, Glostrup Denmark) or a collagen I antibody (LF-67). For collagen I staining in paraffin sections, slides were treated with 3% hydrogen peroxide prior to antigen retrieval with Target Retrieval Solution, pH 9 (DAKO, Carpinteria, Calif.). The slides were then treated with 0.05% trypsin before the primary collagen I antibody was applied at a dilution of 1:500. Sections stained with collagen I or HSV were imaged with a light microscope.

### PCR Analysis

RNA was extracted using an RNeasy mini kit (Qiagen, Valencia, Calif.) and converted to cDNA using an RT² first strand kit (SuperArray Biosciences Corporation, Frederick, Md.). The cDNA quality and concentration were measured with an ND-200 Spectrophotometer (Nanodrop Technologies, Wilmington, Del.). For the PCR reaction, cDNA from all samples were standardized to 1 μg/μl. The reaction was performed with a HotStarTaq Plus DNA Polymerase (Qiagen, Valencia, Calif.). For AGTR1 primers, the inventors used: forward primer—GTCCCGCCTTCGACGCACAA (SEQ ID NO: 1), reverse primer—GGGGCGGTAGGAAAGCGTGC (SEQ ID NO: 2).

### Ki67 Staining, Imaging and Analysis

Ki67 staining was done on paraffin sections 21 days after HSV injection. Slides were microwave processed with Target Retrieval Solution (DAKO, Carpinteria, Calif.) prior to primary antibody detection. The entire tumor section was imaged at 2× magnification and reconstituted as a mosaic. Twenty regions were randomly selected in each tumor. The fraction of Ki67 positive cells in each region was determined by manual count.

### DOXIL® Treatment and Tumor Growth Delay

Two weeks after the implantation of orthotopic pancreatic L3.6PL tumors, mice were randomly selected for losartan or saline treatment. A sub-anti-tumor dose of DOXIL® (4 mg/kg) was infused i.v. via the tail vein after two weeks of losartan treatment (20 mg/kg/day). One week after the DOXIL injection, the tumors were resected and measured.

### Virus Treatment and Tumor Growth Delay

Scid mice bearing subcutaneous HSTS26T and MU89 tumors were randomly divided into control and losartan treated groups. Each arm (control and treated) was subsequently divided into HSV treated and non-HSV treated groups. Tumors that had reached 60 mm³ after two weeks were selected for i.t. HSV injections. Tumors were treated with 10 μl i.t. injections of either PBS or 2.5×10⁵ transducing units (t.u.) of oncolytic HSV MGH2 (gift from E. Antonio Chiocca, Ohio State University, Columbus, Ohio). Two i.t. injections of oncolytic HSV separated by 24 hrs were administered. The injections were done with a Harvard Apparatus Standard Pump 22 infusion/withdraw syringe pump system (Holliston, Mass.) at a flow rate of 4 μl/min. Tumors were measured every 2 to 3 days. Tumor volume was estimated as: V=AB²/2, where V is the tumor volume; A and B are the maximum and minimum diameters of the tumor as measured with calipers.

### Statistics

All the animal experiments were conducted with at least 6 mice in each treatment arm. The tumor growth delay studies in HSTS26T and MU89 tumors were done with at least 8 mice in each group. The rational for the number of mice used was based on power calculations in the inventors' previous studies (McKee T D, et al. (2006) CancerRes 66:2509-2513 and Mok W, et al. (2007) Cancer Res 67:10664-10668), which showed that the inventors needed at least 8 mice in each group to reach statistical significance (p<0.05). All statistical analyses involving two groups were done using a Student's t-test. A p-value lower than 0.05 was considered significant. For multiple groups, a one-way Anova test followed by a Tuskey's post-hoc test was used to determine statistical significance between groups. Statistical significance in figures is identified by an asterix (“*”).

### Example 7

### Angiotensin Blockade Improves Drug Delivery by Normalizing the Tumor microenvironment

Advances in biomedical research have led to the introduction of several novel systemically-administered molecular and nanotherapeutics in both preclinical and clinical settings (Jones, D. (2007) Nat Rev Drug Discov 6, 174-175; Moghimi, S. M. et al. (2005) Faseb J 19, 311-330). While these new agents act on unique targets that afford greater specificity to tumor cells or improved pharmacodynamic properties, their effectiveness suffers from limitations in their delivery owing to the properties of the tumor microenvironment (Jain, R. K. (1998) Nat Med 4, 655-657; Sanhai, W. R. et al. (2008) Nat Nanotechnol 3, 242-244). Growth-induced mechanical forces compress and collapse blood vessels limiting tumor perfusion, while abnormal vasculature leads to heterogeneous drug extravasation. A third determinate of delivery—interstitial transport through tissues—is particularly hindered for nanomedicine in tumors by an abnormally dense and tortuous tumor interstitium (Jain, R. K. & Stylianopoulos (2010) Nat Rev Clin Oncol. 139; Chauhan, V. P. et al. (2009) Biophysical journal 97, 330-336). These barriers impact therapy, particularly, for patients with desmoplastic, fibrotic tumors, including pancreatic (Olive, K. P. et al. (2009) Science 324, 1457-1461), colorectal (Halvorsen, T. B. & Seim, E. (1989) J Clin Pathol 42, 162-166), lung and breast cancer (Ronnov-Jessen, L. et al. (1996) Physiol Rev 76, 69-125)—limiting the amount of drug that reaches the target cancer cells leading to poor effectiveness. Currently, there are limited approaches to overcome these delivery barriers for nanotherapeutics and low molecular weight drugs. Previously, the inventors discovered that relaxin can improve transport through the tumor matrix, but may not facilitate the delivery of low MW agents (B. Seed and R. K. Jain. “Methods to Potentiate Cancer Therapies”, U.S. Pat. No. 6,719,977, Apr. 13, 2004.)

Presented herein is a class of FDA-approved agents that can normalize the tumor microenvironment and improve delivery of both low and high molecular weight drugs. Specifically, the inventors showed that angiotensin blockade “normalizes” interstitial matrix in solid tumors, including breast and pancreatic tumors (FIG. 17A). The inventors assessed whether FDA-approved angiotensin receptor blockers (ARBs) and angiotensin converting enzyme inhibitors (ACE-Is), through this mechanism, can alter the tumor microenvironment to enhance drug delivery. The inventors also determined that ARBs and ACE-Is can decompress blood vessels to improve perfusion (FIGS. 17B-17D), increase tumor hydraulic conductivity to repair vessel function (FIG. 18B), and decrease interstitial matrix density to enhance penetration of nanotherapeutics (FIG. 18D). These agents improve delivery of molecules as small as oxygen—a radiation and chemo sensitizer—through vascular normalization (FIGS. 18A-18B), while also enhancing the penetration of larger agents through interstitial matrix normalization (FIGS. 18C-18D). Through this repair of the entire tumor microenvironment, these agents enhance the effectiveness of low molecular weight chemotherapeutics as well as nanotherepeutics in breast and pancreatic cancer models—leading to reduced tumor growth and longer animal survival (FIGS. 19A-19E). The inventors showed that ARBs and ACE-Is can enhance the delivery of therapeutics, and thus have broad applicability for combination therapy with all classes of anti-cancer agents including small-molecule chemotherapeutics, biologics, and nanoparticle therapies.

Angiotensin blockers offer numerous advantages over other approaches. Anti-angiogenic therapies normalize the vasculature alone and have been approved for only a limited number of indications. Meanwhile, ARBs and ACE-Is are FDA-approved as anti-hypertensives with manageable adverse effects. Matrix-degrading enzymes, which can normalize the collagen matrix, are not selective for tumors and can increase invasion and metastasis. ARBs and ACE-Is generally have no complications associated with matrix remodeling in normal tissues, leading to their safety as anti-hypertensives. ARBs and ACE-Is, as small-molecule agents, can also be delivered via nanovectors containing chemotherapeutics (e.g., liposomes, nano-particles) to enhance their localization to tumors to further limit toxicity. Anti-angiogenics, the only FDA-approved adjuncts that enhance drug delivery to tumors, generally cannot improve delivery for larger particles as they can reduce the size of “pores” in vessel walls. On the contrary, angiotensin blockers presented herein can improve delivery for all classes of anti-tumor diagnostics and therapies.

### Example 8

### In Vitro Screen to Identify Anti-Hypertensive Agents to Lower Collagen in Solid Tumors

This Example provides an assay to rank anti-hypertensive (AH) agents based on their ability to lower collagen I level in tumors.

Since most collagen I is produced by carcinoma-associated fibroblasts (CAFs), a skilled artisan can measure the level of collagen I—along with its molecular determinants [active-TGF-β1, thrombospondin 1 (TSP1) and connective tissue growth factor (CTGF)]—in the supernanants of CAFs after AH treatment.

For example, the inventors determined that losartan reduced TGF-β1 activation and collagen I production in breast CAFs in vitro. Cells were treated with 10 μmol/L of losartan for 24 hrs. Losartan reduced by 90% the active-TGF-β1 levels (p<0.05), while total TGF-β1 levels were unaffected. There was a corresponding 27% decrease in collagen I levels (p<0.05). (See FIG. 1).

### Exemplary Experimental Design:

Anti-hypertensive agents: Any FDA-approved angiotensin receptor blockers (ARBs) can be tested. Exemplary names and doses of these agents can be found via, but not limited to, http://www.globalrph.com/druglist.htm.

Although angiotensin converting enzyme inhibitors (ACEIs) also lower collagen, they do not target the receptor on cells and hence the inventors did not measure their effects on collagen I. Calcium channel blockers can also be evaluated for the collagen lowering effects.

### Cell Culture

Isolate carcinoma-associated fibroblasts (CAFs) from human cancer biopsies using a previously described protocol (Orimo A, et al. (2005) Cell 121(3):335-348). CAFs should be plated in 24 well plates at a concentration of 500K cells/well and allowed 24 hrs to adhere to the plates before the addition of anti-hypertensive drug. For example, all the losartan studies were performed at 10 smol/l for 24 hrs, based on a published protocol (Schuttert J B, et al. (2003) Pflugers Arch 446(3):387-393). Treatment can be done in low serum to reduce background collagen levels. Conditioned medium can be collected at the end of the 24-hr treatment period and analyzed for total and activated TGF-β1, TSP-1, CTGF and collagen levels.

### Protein Assays

In the losartan study (Schuttert J B, et al. (2003) Pflugers Arch 446(3):387-393), Collagen I measurements were done with a type I C-terminal collagen propetide Enzyme Linked Immunosorbent Assay (ELISA) kit (Quidel, San Diego, Calif.) and the Sircol soluble collagen assay (Biocolor Ltd., United Kingdom). TGF-β1 assays were performed with a human TGF-β1 ELISA kit (R&D Systems, Minneapolis, Minn.). The assay only measures the free-form of mature TGF-β1. To measure total levels of TGF-β1 the latent form of TGF-β1 was activated with 1N HCl. TSP-1 assays were performed with a human TSP-1 ELISA kit (R&D Systems, Minneapolis, Minn.). CTGF ELISA kit can be purchased from Leinco (www.leinco.com).

### Protocol Summary:

Isolate or purchase carcinoma associated fibroblasts (CAFs) from breast, pancreatic, and colon carcinomas;

Co-culture CAFs and cancer cells in media containing angiotensin I and ACE;

Treat CAFs for 48 hrs with, for example, 6 doses of an AGTR1 or ACE inhibitor;

Collect supernatant and measure TGF 1, connective tissue growth factor (CTGF), thrombospondin 1, and/or collagen I by ELISA ELISA measurements should be repeated 3 times or more.

Additional Exemplary Testing:

a) Confirm in vitro findings in vivo in a limited number of tumor models.

b) Identify the features of the AH that make them more effective modifiers of collagen to screen for new AHs.

### Example 9

### Combination of Angiotensin Blockade with Inhibition of Alternate Profibrotic Pathways to Improve Drug Delivery to Tumors

The inventors have discovered that normalization of the interstitial matrix through angiotensin signaling blockade improves drug delivery, at least partly, through two mechanisms: it relaxes the inherent compressive force in tumors to improve vessel perfusion, and it reduces the viscoelastic and steric hindrance on drug transport directly imparted by the matrix. Angiotensin signaling blockade can safely inhibit activation of the profibrotic TGF-beta and CTGF pathways downstream to produce these changes. In some embodiments, partnering angiotensin blockers with inhibitors of profibrotic pathways that are independent of TGF-beta and CTGF activation—including endothelin-14371236.1 1, PDGF, Wnt/beta-catenin, IGF-1, TNF-alpha, and IL-4—can enhance these effects, further improving drug delivery and effectiveness. For example, endothelin receptor blockers (ERBs) and PDGF inhibitors (PDGF-Is) can be used in combination with angiotensin blockers. ERBs treat pulmonary arterial hypertension and can be used as a class of therapy for cancer (Nelson et al. (2003) Nature Reviews Vol. 3:110-116), for example with angiotensin blockers. PDGF-Is haven been reported for their potential anti-vascular effects in tumors (Baluk et al. (2005) Current Opinion in Genetics & Development 15:102-111, Andrae et al. (2008) Genes & Development 22:1276-1312). Endothelin blockade has been reported to reduce fibrogenesis in the liver (Binder et al. (2009) Mol. Cancer. Ther. 8:2452-2460), lung (Park et al. (1997) Am J. Respir Crit. Care Med. Vol. 156:600-608), and heart through inhibition of TGF-beta synthesis (Ogata et al. (2002) Clinical Science 103 (Suppl. 48):284S-288S), and has been reported to reduce tumor progression and metastasis in tumor models (Nelson et al. supra; Binder et al. supra). Meanwhile, PDGF inhibition have been reported to prevent fibrogenesis in idiopathic pulmonary fibrosis and scleroderma (Grimminger et al. (2010) Nature Reviews Vol. 9:956-970; Andrae et al. (2008) Genes & Development 22:1276-1312), and the inventors have determined that it can reduce collagen levels in tumors (data not shown). ERBs have been reported to be well-tolerated with a potential to improve overall survival in prostate cancer (James et al. (2009) European Urology 55:1112-1123) and non-small cell lung cancer (Chiappori et al. (2008) Clin Cancer Res 14:1464-1469). Accordingly, the combination of an angiotensin blockade with endothelin-1 and/or PDGF blockade—with careful dosing—should produce an additive improvement to drug delivery with minimal additional toxicity. In some embodiments, endothelin-1 and/or PDGF blockade can be used at a sub-therapuetic dose in combination with an angiotensin blockade, which can be used at a sub-anti-hypertensive dose and/or sub-anti-tumor dose, for improved drug delivery and/or treatment of cancer.

### Example 10

### Angiotensin Inhibitors Decompress Tumor Vessels to Enhance Drug Delivery

### Introduction

Retrospective analysis of clinical data shows that the use of angiotensin receptor blockers (ARBs) and angiotensin converting enzyme inhibitors (ACE-Is) to manage hypertension in cancer patients receiving standard therapies associated with longer survival (Keizman D, et al., Eur J. Cancer. 2011; 47(13): 1955-61; Nakai Y, et al., Br J. Cancer. 2010; 103(11): 1644-8; Wilop S, et al., Journal of Cancer Research and Clinical Oncology. 2009; 135(10): 1429-35). However, a causal relationship between the use of ARBs/ACE-Is and clinical benefit, as well as the mechanism behind this potential effect, has not been demonstrated.

From the retrospective studie described above, it is unclear whether ARBs and ACE-Is directly potentiate standard therapies, and if so, if they have synergistic or additive effects. It is assumed that their indirect anti-angiogenic properties, through downstream VEGF inhibition (George A J, et al., Nat Rev Cancer. 2010; 10(11): 745-59), benefit survival additively. However, malignant tumors, and in particular pancreatic cancers, have a notoriously poor blood perfusion. Indeed, a specific anti-VEGF inhibitor (the antibody bevacizumab) showed no overall survival benefit when added to chemotherapy in randomized, double-blind, phase-III trials for breast, pancreatic, kidney and non-small cell lung cancers (Escudier B, et al., Journal of Clinical Oncology. 2010; 28(13): 2144-50; Kindler H L, et al., Journal of Clinical Oncology. 2010; 28(22): 3617-22; Miles D W, et al., Cancer research. 2009; 69(24): 495S; Reck M, et al., Ann Oncol. 2010; 21(9): 1804-9).

On the other hand, angiotensin inhibition can also modulate the non-vascular tumor microenvironment (Table 1). Therefore, angiotensin inhibitors could synergistically enhance chemotherapy by increasing drug and oxygen delivery to the tumor. Chemotherapy effectiveness is dependent on drug delivery (Jain R K. Science. 2005; 307(5706): 58-62). Furthermore, inadequate oxygen delivery can lead to hypoxia-induced drug resistance (Wilson W R, et al., Nat Rev Cancer. 2011; 11(6): 393-410), selection for aggressive cells (Harris A L. Nature reviews. 2002; 2(1): 38-47), and immune suppression in tumors (Facciabene A, et al., Nature. 2011; 475(7355): 226-U141). By alleviating hypoxia and improving drug delivery, this mechanism would position ARBs and ACE-Is as a new class of safe anti-cancer adjuncts.

TABLE 1
Effects of angiotensin activation/blockade on the tumor microenvironment

| | | |
|---|---|---|
| angiotensin II (activation) | ↑ tumor growth (1) | ↑ fibroblast proliferation (2) |
| | | ↑ fibroblast activation (3) |
| angiotensin II receptor 1^{-/-} mice | | ↓ tumor growth (4) |
| angiotensin II receptor 2^{-/-} mice | | ↑ fibrosis (5) |
| | | ↑ tumor growth (6) |
| ARBs and ACE-Is (blockade) | ↓ tumor growth (7) | ↓ tumor growth (9) |
| | ↓ stromal matrix production (8) | ↓ stromal matrix production (8) |
| | | ↓ fibroblast proliferation (10) |

(1) Greco S, et al., Journal of Cellular Physiology. 2003; 196(2): 370-7.
(2) Hama K, et al., Biochemical and Biophysical Research Communications. 2006; 340(3): 742-50.
(3) Hama K, et al., Biochem Biophys Res Commun. 2004; 315(4): 905-11.
(4) George AJ, et al., Nat Rev Cancer. 2010; 10(11): 745-59.
(5) Ulmasov B, et al., Am J Physiol Gastrointest Liver Physiol. 2009; 296(2): G284-94.
(6) Doi C, et al., BMC Cancer. 2010; 10(1): 67.
(7) Rhodes DR, et al., Proc Natl Acad Sci USA. 2009; 106(25): 10284-9.
(8) Diop-Frimpong B, et al., Proc Natl Acad Sci USA. 2011; 108(7): 2909-14.
(9) Arnold SA, et al., PLoS One. 2012; 7(2): e31384.
(10) Liu WB, et al., World J Gastroenterol. 2005; 11(41): 6489-94.

Drug and oxygen delivery is dependent solely on perfused vessels. Solid stress (Guyton A C, et al., Physiological Reviews. 1971; 51(3): 527), which accumulates in tumors due to unbridled cell proliferation and matrix production in a confined microenvironment (Helmlinger G, et al., Nat. Biotechnol. 1997; 15(8): 778-83; Stylianopoulos T, et al., Growth-induced mechanical stress in murine and human tumors: causes, consequences and remedies. under review. 2012; Roose T, et al., Microvasc Res. 2003; 66(3): 204-12), collapses blood vessels and limits perfusion (Padera T P, et al., Nature. 2004; 427(6976): 695). This impacts therapy by reducing the drug doses that reach cancer cells, hindering effectiveness. Indeed, patients with low tumor perfusion, presumably with hypoxia and low drug delivery, show poorer chemotherapy responses and shorter survival versus patients with high perfusion (Sorensen A G, et al., Cancer research. 2012; 72(2): 402-7; Park M S, et al., Radiology. 2009; 250(1): 110-7). Most solid tumors in patients have hypoxic regions and collapsed vessels, e.g., breast cancer (FIG. 20) and pancreatic cancer (Olive K P, et al., Science. 2009; 324(5933): 1457-61). Therefore, this example illustrates the hypothesis that angiotensin inhibitors may increase perfusion. As described in this example, this hypothesis was tested in orthotopic breast and pancreatic tumor models using the ARB losartan.

### Results

To assess how losartan affects blood vascular perfusion in orthotopic breast and pancreatic tumors, mice were injected with lectin as a marker for perfused (patent and functional) vessels before tumor excision, and then immunostained tissue sections with an anti-CD31 antibody, which marks both perfused and non-perfused vessels (Olive K P, et al., Science. 2009; 324(5933): 1457-61). These tumors were found to be severely hypo-perfused (FIG. 21): only 23% of vessels in E0771 breast tumors and 21% in AK4.4 pancreatic tumors were perfused with blood (FIGS. 17D and 22A). A 40 mg/kg dose of losartan, which does not decrease blood pressure in tumor-bearing mice (FIG. 23), significantly improved the perfused vessel fraction to 43% in E0771 and 45% in AK4.4 (FIGS. 17D and 22A). Losartan treatment increased vessel diameters in E0771 (FIG. 22B), suggesting vascular decompression as the mechanism of action. Despite the previous classification of ARBs and ACE-Is as anti-angiogenics (George A J, et al., Nat Rev Cancer. 2010; 10(11):745-59), losartan did not affect the CD31+ vessel density in these tumors (FIGS. 22C-22D). Thus, losartan can increase blood supply in tumors by opening existing collapsed blood vessels.

The mechanism responsible for the increase in tumor vessel perfusion by losartan was investigated. Desmoplasia, high stromal cell and matrix density, is a major contributor to the solid stress accumulation that compresses vessels (Stylianopoulos T, et al., Growth-induced mechanical stress in murine and human tumors: causes, consequences and remedies. under review. 2012). Since angiotensin inhibitors reduce matrix production in tumors (Diop-Frimpong B, et al., Proc Natl Acad Sci USA. 2011; 108(7): 2909-14), it was hypothesized that they decompress vessels and increase perfusion by decreasing solid stress. Collagen levels were used as a metric of matrix production as described in Diop-Frimpong et al., Proc Nall Acad Sci USA. 2011; 108(7): 2909-14, and losartan was found to reduce the collagen I levels in E0771 and AK4.4 tumors (FIGS. 24A and 24C). Moreover, losartan decreased collagen I concentration, measured based on staining intensity, in E0771 and AK4.4 tumors (FIGS. 24B-24C). Of note, dense collagen seemed to colocalize with low-perfusion areas (FIG. 24C). Next, solid stress in these tumors was measured using recently established technique described in Stylianopoulos T, et al., Growth-induced mechanical stress in murine and human tumors: causes, consequences and remedies. under review. 2012. Losartan reduced solid stress in both E0771 and AK4.4 tumors (FIG. 24D), as well as in 4T1 breast and Pan-02 pancreatic tumors (FIG. 25). Similarly, the ACE-I lisinopril reduced collagen I levels in E0771 and AK4.4 tumors and solid stress in E0771 tumors (FIG. 26), indicating that the mechanism of action is via angiotensin signaling inhibition rather than an off-target effect of losartan. Furthermore, a panel of ARBs were tested, and they all reduced solid stress (FIG. 27). These data show that angiotensin blockers can indirectly improve vascular perfusion in desmoplastic tumors by decompressing blood vessels through their anti-matrix effects.

Since drug and oxygen delivery is chiefly controlled by vascular perfusion, the effects of losartan on delivery to tumors were tested. A mathematical approach was used to analyze the efficiency of the vascular network for delivery. Using multiphoton microscopy, the perfused vessel networks of E0771 tumors were imaged (FIG. 17D). Based on fractal analysis and metrics of intervascular spaces (Baish J W, et al., Proc Natl Acad Sci USA. 2011; 108(5): 1799-803), it was found that losartan reorganizes networks toward the structure of normal capillary beds and reduces the maximum distance drugs and oxygen must travel to reach tumor cells (FIGS. 28A-28B).

The accumulation of the small-molecule chemotherapeutic fluorouracil (5-FU) was then measured. Losartan improved 5-FU delivery to AK4.4 tumors, while not affecting delivery to normal organs (FIG. 29A). These data imply that this strategy for enhancing delivery selectively affects tumors because solid stress does not accumulate in normal organs.

Oxygenation was then studied using phosphorescence quenching microscopy. It was found that losartan treatment maintained tumor oxygen levels in E0771 tumors, while control-treated tumors showed a typical growth-dependent drop in oxygenation (FIGS. 29B-29C). This decrease in tumor hypoxia was confirmed by using pimonidazole staining (FIGS. 29D and 30). To determine if losartan also increases nanomedicine delivery, which is dependent on both vascular supply and penetration across vessel walls into tumor tissue, nanoparticle penetration was measured using intravital multiphoton microscopy. Nanoparticle penetration rates were quantified as transvascular mass flux per unit vascular surface area and transvascular concentration difference, termed as the effective permeability. It was found that losartan improved effective permeability for 12 nm, 60 nm, and 125 nm nanoparticles (FIG. 29E), i.e., for the entire size range of nanomedicines in clinical use. Penetration rates are largely dependent on fluid flow through the viscoelastic and tortuous tumor interstitium, which is a function of interstitial hydraulic conductivity. It was determined that losartan also increases the hydraulic conductivity of E0771 tumors (FIG. 31), likely through its anti-matrix effects, and a mathematical model (Chauhan V P, et al., Nature Nanotechnology. 2012; advance online publication) was used to confirm that this can improve the penetration rate of all sizes of nanomedicines (FIG. 32). Thus, “microenvironmental normalization” with ARBs and ACE-Is increases the delivery of oxygen and all sizes of therapeutics to tumors.

Given these effects on delivery, angiotensin inhibitors could act as adjunct therapies to synergistically improve the effectiveness of small-molecule chemotherapeutics. Thus, losartan was tested in combination with doxorubicin in E0771 and 4T1 tumors, or with 5FU in AK4.4 tumors. It was found that whereas losartan or doxorubicin given alone had no significant effect on tumor growth rate, the combination significantly delayed E0771 and 4T1 tumor growth (FIGS. 19A-19B and 35). Similarly, while losartan or 5-FU monotherapy had no significant effect on tumor growth rate in the highly chemoresistant AK4.4 tumors, their combination greatly slowed tumor growth (FIG. 33). Moreover, the combination of losartan and doxorubicin increased median survival of mice bearing E0771 tumors from 12 days with saline alone to 23.5 days, compared with only 16 days with doxorubicin alone (FIG. 19C). Similarly, the combination improved survival in mice with 4T1 tumors (FIG. 35). Importantly, losartan alone, despite increasing blood perfusion in tumors, did not shorten survival in mice bearing E0771, 4T1 or AK4.4 tumors (FIGS. 19C, 34-35). Furthermore, losartan monotherapy did not increase metastasis in AK4.4, and its combination with 5-FU appeared to reduce the incidence and size of metastases (Table 2). Together, these data demonstrate that angiotensin inhibitors can improve the effectiveness of small molecule chemotherapeutics through anti-matrix effects.

TABLE 2
Angiotensin inhibitors do not affect metastasis in aggressive mouse models
Disease dissemination at death of mice bearing AK4.4 pancreatic tumors

| | | | | | | | | | |
|---|---|---|---|---|---|---|---|---|---|
| Saline | Saline | + | +, discolored | − | − | − | + | − | − |
| Saline | Saline | ++ | discolored | − | − | − | − | ++ | + |
| Saline | Saline | ++ | − | − | − | − | + | ++ | + |
| Saline | Saline | ++ | discolored | + | − | − | − | ++ | + |
| Saline | Saline | ++ | discolored | − | − | − | + | ++ | + |
| Saline | Saline | ++ | − | + | − | − | + | ++ | + |
| Losartan | Saline | ++ | discolored | − | − | − | − | ++ | + |
| Losartan | Saline | ++ | − | − | − | − | − | ++ | + |
| Losartan | Saline | ++ | − | − | − | − | + | + | + |
| Losartan | Saline | + | discolored | + | − | − | + | + | + |
| Losartan | Saline | ++ | discolored | + | − | − | + | ++ | + |
| Saline | 5-FU | + | discolored | − | − | − | − | − | − |
| Saline | 5-FU | ++ | discolored | − | − | − | − | ++ | + |
| Saline | 5-FU | ++ | +, discolored | − | − | − | − | ++ | + |
| Saline | 5-FU | + | discolored | + | − | − | − | + | + |
| Saline | 5-FU | ++ | discolored | − | − | − | − | ++ | + |
| Saline | 5-FU | + | discolored | − | − | − | + | ++ | + |
| Saline | 5-FU | + | − | − | − | − | + | + | + |
| Losartan | 5-FU | ++ | discolored | + | − | − | − | ++ | + |
| Losartan | 5-FU | ++ | − | − | − | − | − | ++ | + |
| Losartan | 5-FU | + | − | − | − | − | − | + | + |
| Losartan | 5-FU | + | − | − | − | − | + | + | + |
| Losartan | 5-FU | + | − | + | − | − | − | + | + |
| Losartan | 5-FU | + | +, discolored | + | − | − | − | + | + |
| Losartan | 5-FU | + | − | + | − | − | − | + | + |

− no metastases,
+ macrometastases present,
++ large macrometastases ( ).

### Discussion

Several attempts at developing adjunct therapies to enhance drug and oxygen delivery have been made in the past. For example, anti-VEGF therapies shrink vessel pores (Chauhan V P, et al., Nature Nanotechnology. 2012; advance online publication), reduce interstitial fluid pressure (IFP) (Tong R T, et al., Cancer research. 2004; 64(11): 3731-6); Goel S, et al., Physiol Rev. 2011; 91(3): 1071-121) and increase perfusion in patients (Sorensen A G, et al., Cancer research. 2012; 72(2): 402-7). This “vascular normalization” approach is suited to well-perfused tumors (Sorensen A G, et al., and Goel S, et al.), but may not work in desmoplastic tumors such as in pancreatic cancer when a significant fraction of tumor vessels are collapsed. Moreover, most anti-angiogenic drugs lead to hypertension in a significant number of cancer patients and this hypertension is currently managed with a variety of anti-hypertensive drugs (Keizman D, et al., Eur J. Cancer. 2011; 47(13): 1955-61.). Another example is anti-hyaluronan enzymatic therapy, which can also increase vessel diameter in pancreatic tumors (Provenzano Paolo P, et al., Cancer Cell. 2012; 21(3): 418-29.), possibly through decompression (Stylianopoulos T, et Growth-induced mechanical stress in murine and human tumors: causes, consequences and remedies. under review. 2012). Though this strategy is quite promising, it is limited to tumors with high hyaluronan levels and may lead to hyaluronan degradation in normal organs. Anti-Hedgehog pathway treatment with IPI-926 increases vessel density in pancreatic tumors (Olive K P, et al., Science. 2009; 324(5933): 1457-61), presumably by reducing stromal cell density and hence solid stress (Stylianopoulos T, et al., Growth-induced mechanical stress in murine and human tumors: causes, consequences and remedies. under review. 2012). While this strategy is attractive for these poorly perfused tumors, IPI-926 failed in a recent randomized phase II clinical trial. Finally, co-administration of therapeutics with the peptide iRGD improves drug penetration in tumors by an active transport mechanism (Sugahara K N, et al., Science. 2010; 328(5981): 1031-5). Still, this does not address the problem of poor vascular supply and thus its effect in desmoplastic, hypovascularized tumors is not known.

ARBs and ACE-Is would be compatible with all these strategies, due to the differences in their targets and mechanisms. Moreover, in contrast to the other drugs and agents that can improve drug delivery, ARBs and ACE-Is are the only FDA-approved drugs that can reduce solid stress thus far. Despite this great promise, ARBs and ACE-Is may be limited in usage by several factors. Notably, the activity of these drugs should be most effective in desmoplastic tumors, such as in breast, stomach, and pancreatic cancer. In addition, not all breast tumors are desmoplastic, which could explain how the disease is sometimes curable with chemotherapy, and thus not all patients may be candidates for ARBs and ACE-Is. Additionally, these agents will only work in patients with angiotensin-II receptor type-1 positive tumors. Furthermore, ARBs and ACE-Is would be contraindicated for patients with low blood pressure or certain other co-morbidities.

In summary, ARBs and ACE-Is were found to increase the delivery of oxygen, small-molecule chemotherapeutics, and nanomedicine through “microenvironmental normalization” mechanisms in desmoplastic tumors. In comparison, only “vascular normalization,” as with direct and indirect anti-angiogenic therapy (Jain R K. Science. 2005; 307(5706): 58-62), has been shown to increase delivery nearly so comprehensively (Tong R T, et al., Cancer research. 2004; 64(11): 3731-6; Chauhan V P, et al., Nature Nanotechnology. 2012; advance online publication; Goel S, et al., Physiol Rev. 2011; 91(3): 1071-121). Nevertheless, the safety and low cost of these drugs—along with their synergistic improvement of cancer therapy—makes a strong case for repurposing ARBs and ACE-Is as cancer therapies.

### Methods

Drug preparation: Angiotensin inhibitors (losartan, lisinopril, valsartan, and candesartan) were obtained as pills. The pills were crushed using a mortar and pestle and the powder was dissolved in phosphate buffered saline (PBS) over 24 hours. The solutions were then sterile filtered for injection. Doxorubicin and 5-FU were obtained as solutions for injection, and were injected without modification. All drugs were purchased from the pharmacy at Massachusetts General Hospital.

Tumor models: AK4.4 was kindly provided by Dr. Nabeel Bardeesy, and was isolated from mice generating spontaneous pancreatic tumors (Kras^{G12} and p53^{+/-}). Orthotopic pancreatic tumors were generated by implanting a small piece (1 mm³) of viable tumor tissue (from a source tumor in a separate animal) into the pancreas of a male FVB mouse (AK4.4 model) or C57BL/6 (Pan-02 model) mouse. Orthotopic breast tumors were similarly generated by implanting a chunk of viable tumor tissue into the mammary fat pad of a female severe combined immunodeficient (SCID) mouse. All animal procedures were carried out following the Public Health Service Policy on Humane Care of Laboratory Animals and approved by the Institutional Animal Care and Use Committee of Massachusetts General Hospital.

Vessel perfusion, matrix level, and hypoxia histology: For breast tumors, mice bearing orthotopic E0771 were split into treatment groups, time-matched for time after implantation and size-matched for tumor volume at this time (−100 mm³). For pancreatic tumors, mice bearing orthotopic AK4.4 were split into treatment groups, size-matched for tumor volume (˜22 mm³), 6 days after implantation. The mice were then treated with 40 mg/kg losartan or an equal volume of PBS intraperitoneally each day for 6 (E0771) or 7 (AK4.4) days. On the day of the last treatment, mice were slowly (−2 min) injected with 100 μL of 1 mg/mL biotinylated lectin (Vector Labs), administered retro-orbitally 5 min prior to tumor removal. For hypoxia studies, the mice were also injected with 60 mg/kg of 10 mg/mL pimonidazole 1 hr prior to tumor removal. The tumors were then excised, fixed in 4% formaldehyde in PBS (30 min/mm diameter of tissue), incubated in 30% sucrose in PBS overnight at 4° C., and frozen in optimal cutting temperature compound (Tissue-Tek). Transverse tumors sections, 40 μm thick, were immunostained with antibodies to endothelial marker CD31, and counterstained by mounting with DAPI-containing medium (Vector Labs). For matrix staining, collagen I was detected using the LF-67 antibody provided by Dr. Larry Fisher (National Institute of Dental Research, Bethesda, Md.).

Histological image analysis: Eight random images (four interior, four periphery) at 20× magnification were taken from each slide using a confocal microscope (Olympus). For vascular analysis, vessels were skeletonized and segmented using a custom, semi-automated tracing program developed in MATLAB (The MathWorks) allowing the removal of structures under thirty pixels and regions of autofluorescence. For perfusion, the number of vessels counted by this program with colocalization of lectin and CD31 staining was divided by the number of vessels counted with CD31 staining. For vessel metrics, including diameter and density, the program determined the average size of all counted vessels and their length, as well as the count per area Images of collagen I stained sections were analyzed based on the area fraction of positive staining and on the average staining intensity (concentration) in each image. The concentration data were normalized to the average control intensity. Images of hypoxia staining with pimonidazole were similarly analyzed for the area fraction of positive staining, counting the fraction of pixels above a threshold based on background intensity. Identical analysis settings and thresholds were used for all tumors.

Solid stress: Solid stress was measured using the tumor opening technique as described previously (Stylianopoulos T, et al., Growth-induced mechanical stress in murine and human tumors: causes, consequences and remedies. under review. 2012). When the tumors reached a size of −1 cm in diameter, the mice were anesthetized. Subsequently, each tumor was excised, washed with Hanks' Balanced Salt Solution (HBSS) and its three dimensions were measured. Each tumor was cut along its longest axis, to a depth of 80% of its shortest dimension, using a scalpel. The tumors were allowed to relax for 10 minutes in HBSS to diminish any transient, poro-elastic responses. Afterwards, the opening resulting from the cut was measured at the middle of the cut at the surface of the tumor. Solid stress is proportional to the size of the opening relative to the size of the dimension perpendicular to the cut.

Drug delivery: Mice bearing orthotopic AK4.4 were split into treatment groups, size-matched for tumor volume (−22 mm³), 6 days after implantation. The mice were then treated with 40 mg/kg losartan or an equal volume of PBS intraperitoneally each day for 7 days. On the day of the last treatment, mice were injected with 100 mg/kg 5-FU, administered retro-orbitally 30 min prior to tumor and organ removal. The tissue was dabbed of excess blood then snap-frozen in liquid nitrogen for analysis. 5-FU was isolated from the tissues and measured using liquid-liquid extraction followed by reverse-phase high-performance liquid chromatography with tandem mass-spectrometry.

In vivo imaging: For imaging studies, E0771 tumors were implanted in mice bearing mammary fat pad chambers (124) and allowed to grow to −3 mm in diameter. Multiphoton imaging was carried out as described previously (124) on a custom-built multiphoton laser-scanning microscope using confocal laser-scanning microscope body (Olympus 300, Optical Analysis) and a broadband femtosecond laser source (High Performance MaiTai, Spectra-Physics). Images were taken at ˜60 mW at sample surface. Mosaic images were taken in raster pattern using a motorized stage (H101, Prior Scientific) and customized automation software (LabView, National Instruments). Imaging studies were performed with a 20× magnification, 0.95NA water immersion objective (Olympus XLUMPlanFl, 1-UB965, Optical Analysis).

Tissue oxygenation: p02 in the tumors was measured using phosphorescence quenching microscopy (Helmlinger G, et al., Nature medicine. 1997; 3(2):177-82), which was adapted to multiphoton microscopy (Sakadzic S, et al., Nature Methods. 2010; 7(9): 755-U125). An oxygen-sensitive porphyrin, Oxyphor R2 (Oxygen Enterprises) was injected retro-orbitally 12 hrs prior to imaging, and was reinjected immediately prior to imaging along with 2MDa FITC-dextran (Sigma-Aldrich) for functional vascular tracing. A mosaic image of the tumor was collected, and oxygen was then measured in an evenly spaced 12×12 grid at four depths (60, 120, 180, and 240 μm) in the tumor. At each point in the grid, the phosphorescence lifetime of the probe was measured after each of several repeated brief intense pulses of 1020 nm laser light, and these lifetime measurements were combined. A two-component model was used to calculate the oxygen tension from each lifetime measurement, accounting for binding and quenching of the probe by both oxygen and proteins. The grid was then overlayed on the mosaic images to make an oxygen map.

Nanoparticle penetration: A mixture of nanoparticles with diameters of 12 nm (476 nm emission), 60 nm (540 nm emission), and 125 nm (625 nm emission) was prepared for intravenous injection as described previously (Popovic Z, et al., Angew Chem Int Ed Engl. 2010; 49(46): 8649-52). Concentrations were adjusted with in vitro calibration to result in roughly equal photoluminescence intensity for all three nanoparticle samples under 800 nm multiphoton excitation. Following retro-orbital injection of 200 μL with these concentrations, multiphoton imaging was carried out as described above at depths from 0-201 μm, with 2.76 μm steps and 2.76×2.76 μm pixels. Images were taken every 3 min at each region of interest for a duration of 1 hr. Images were analyzed using custom analysis software developed in Matlab (The Mathworks) as described previously (Chauhan V P, et al., Nature Nanotechnology. 2012; advance online publication; Chauhan V P, et al., Angewandte Chemie International Edition. 2011; 50(48): 11417-20). The analysis approach involved 3D vessel tracing to create vessel metrics and a 3D map of voxel intensity versus distance to the nearest vessel over time. Images were also corrected for sample movement over time with 3D image registration. The effective permeability was calculated with

$$\frac{k}{{S}_{v} ( {C}_{v} - C )} = {P}_{eff} = {lim}_{t \to 0} \frac{\partial }{\partial t} \frac{{\int }_{r = R}^{∞} C ( r ) r d r}{( {C}_{v} - C ) R} ,$$

where Jₜ is the transvascular flux, Sᵥ is the vessel surface area, C. is the concentration of the probe in the vessel, C is the concentration of the probe immediately extravascular, P_{eff} is the effective permeability (Chauhan V P, et al., Nature Nanotechnology. 2012; advance online publication; Chauhan V P, et al., Angewandte Chemie International Edition. 2011; 50(48): 11417-20), t is time after the initial image, r is the distance from the vessel central axis, and R is the vessel radius at that point along the vessel. Fluorescence intensities were used as these concentrations. The calculation was made as an average over the entire imaged volume for each tumor.

Breast tumor growth and survival studies: Mice bearing orthotopic E0771 or 4T1 breast tumors were split into treatment groups, time-matched for time after implantation and size-matched for tumor volume at this time (110-111 mm³ in E0771, in 4T1). The mice were treated at this initial size with 40 mg/kg losartan or an equal volume of PBS intraperitoneally on day 0 and each subsequent day. The mice were then treated with either 2 mg/kg doxorubicin or an equal volume of saline by intraperitonteal injection every three days beginning on day 1 (after 2 losartan or PBS treatments). The primary tumors were then measured every 2 days, beginning on day 0, using calipers. Tumor growth was quantified using the time for each to reach double its initial volume Animal survival was quantified based on time of death after initiation of treatment or time to reach excessive tumor burden (>1000 mm³).

Pancreatic tumor growth and metastasis studies: Mice bearing orthotopic AK4.4 pancreatic tumors were split into treatment groups, size-matched for tumor volume (22 mm³), 6 days after implantation. The mice were treated with 40 mg/kg losartan or an equal volume of PBS intraperitoneally on day 7 after implantation and each subsequent day. The mice were then treated with either 60 mg/kg 5-FU or an equal volume of saline by intravenous injection on days 9 and 13 after implantation. Tumors were extracted on day 14 for measurement using calipers. Tumor growth was quantified using the size at day 14. For metastasis studies, mice were treated with losartan or PBS on day 11 after implantation, then with 5-FU or saline on days 13 and 17. Metastatic burden was assessed at death.

Mean arterial blood pressure: Mice bearing orthotopic AK4.4 pancreatic tumors were used for blood pressure measurements. Mean arterial blood pressure was measured by cannulation of the left carotid artery after a longitudinal skin incision above the trachea, as described previously (Zlotecki R A, et al., Microvasc Res. 1995; 50(3): 429-43). After removal of the submandibular gland, the paratracheal muscles was split and the left carotid artery was isolated. The cranial end of the artery was ligated with a 6-0 silk suture and another suture was tied loosely around the central part of the artery. A metal clamp was then positioned caudally to stop blood flow during the cannulation. A polyethylene catheter (PE-10, Becton-Dickinson) filled with heparinised saline was then be inserted through a hole cut proximally to the cranial ligature, and the other suture was tied tightly around the tubing and artery. The clamp was then removed and the end of the tubing was connected to a pressure transducer for the measurement of blood pressure.

Mathematical analysis and modeling: The analysis was carried out on mosaic images of whole tumors taken with multiphoton microscopy after injection of 2MDa FITC-dextran as a perfused vessel tracer. Details of the models and corresponding equations are described in Chauhan V P, et al., Nature Nanotechnology. 2012; advance online publication, and Baish J W, et al., Proc Natl Acad Sci USA. 2011; 108(5): 1799-803.

Hydraulic conductivity: Mice bearing orthotopic E0771 breast tumors were split into treatment groups, time-matched for time after implantation and size-matched for tumor volume at this time (−100 mm³). The mice were then treated with 40 mg/kg losartan or an equal volume of PBS intraperitoneally each day for 6 days. Interstitial hydraulic conductivity was measured as described previously (Mok W, et al., Cancer Res. 2007; 67(22): 10664-8; Wabb E A, et al., Cancer research. 1974; 34(10): 2814-22). The tumors were excised, and a 3 mm biopsy punch was used to cut a cylindrical tissue block from each. A scalpel was then used to cut a 1.7 mm-thick disc of viable tumor tissue from this cylindrical block. The disc-shaped tissue block was then placed into a clamp with a fluid flow channel. A pressure head of 10 cmH20 was applied, and a small bubble was created to measure the fluid velocity in the 0.58 mm diameter tubing connecting the pressure head to the clamp. Measurements were taken over 5-10 min per tumor. The interstitial hydraulic conductivity was then calculated as

$$\frac{Q}{A} = - K \frac{Δ p}{Δ x} ,$$

where Q is the volumetric flow rate through the tissue, A is the cross-sectional area of the tissue block, K is the interstitial hydraulic conductivity, Ap is the applied pressure drop, and Ax is the tissue block thickness.

### Example 11

### Losartan Pharmacokinetic/Pharmacodynamic (PK/PD) Analysis in Mouse Pancreatic Tumor Model

This example presents a study that compared the pump administration and pulsatile injections of losartan based on the results of PK/PD analysis in mouse AK4.4 pancreatic tumor model.

Study Design Summary

Species and sex: Sixty male FVB mice.

Time points: Day 14 and 19 (2 hours and 6 hours post injection for blood and tumor sample collection)

Compounds: Losartan

Vehicle: Phosphate buffered saline (PBS)

Concentration: Losartan for subcutaneous injection without pump (40 mg/kg), losartan for subcutaneous pump administration (40 mg/kg/day)

Route of administration: subcutaneous (SC) administration for losartan and PBS; intravenous (IV) injection for lectin

Procedures: AK4.4 pancreatic tumors from donor mice were implanted into the pancreas of sixty mice on Day 1. On day 7 the tumors were all measured, and then the mice were randomized into one group of sixteen mice for subcutaneous injection of PBS as a control (Group 1), one group of twenty-two mice for subcutaneous pump administration of losartan (Group 2), and one group of twenty-two mice for subcutaneous injection of losartan in the absence of pump. Subcutaneous pumps with losartan were implanted on day 7 into the twenty-two mice in Group 2. All of the mice in Group 1 were subcutaneously dosed with PBS daily until day 14 or day 19. All of the mice in Groups 2 and 3 were dosed with losartan at 40 mg/kg daily until day 14 or day 19. Blood and tumor samples were collected either 2 hours or 6 hours post injection. The body weight was measured for all mice every other weekday (i.e., every Monday, Wednesday, and Friday) until the final dose on day 14 or 19. For PK/PD analysis, six mice in each group (three mice per time point in Groups 2 and 3) were euthanized on day 14 and day 19. Blood samples were collected and tumor weight was measured. To perform collagen I immunohistochemistry, the tumors of ten mice were collected following the measurement of the tumor volume.

Sample Collection

Sample type: plasma and weighed tumors (for PK/PD analysis) and tumors (for immunohistochemistry analysis)

Collection instruction: For PK/PD analysis, blood samples from euthanized mice were collected into lithium heparin tubes and inverted until centrifuged for 5 minutes in 4° C. at 6,000 rpm. Weighed tumors were homogenized with PBS. For immunohistochemistry analysis of collagen I, tumors were collected and sliced into two pieces. Both pieces were placed in 4% formaldehyde for up to 6 hours (0.5 hour per 1 mm) then rinsed three times with PBS. The tumor samples were stored in 30% sucrose for 24 hours and then frozen in 30×24 mm molds with optimum cutting temperature compound (OCT). The tumor samples were then stored in tissue cassettes at −80° C.

Sample storage: All samples were stored at −80° C.

### Results

### Losartan Bioanalysis

The plasma and tumor levels of losartan and losartan carboxylic acid (E3174) were measured in Group 2 (subcutaneous pump administration at 40 mg/kg) and Group 3 (subcutaneous injections without pump at 40 mg/kg) mice on day 14 and day 19. The concentration of losartan and losartan carboxylic acid in plasma and tumor was quantified using high performance liquid chromatography/fluorescence (HPLC/FLU) method.

As shown in Table 3, the plasma and tumor losartan levels were higher in the mice administered with losartan via subcutaneous pump (Group 2, 6-hour time point), as compared to the mice injected with losartan subcutaneously without pump (Group 3, 6-hour time point), on both day 14 and day 19. Similarly, the plasma and tumor levels of losartan were higher in the mice administered with losartan via subcutaneous pump (Group 2, 2-hour time point), as compared to the mice injected with losartan subcutaneously without pump (Group 3, 2-hour time point), on day 19 (Table 3). Table 3 also indicates that the plasma and tumor levels of losartan increased or became steady from the 2-hour time point to the 6-hour time point in the mice administered with losartan via subcutaneous pump (Group 2, both day 14 and day 19), whereas the plasma and tumor levels of losartan generally decreased from the 2-hour time point to the 6-hour time point in the mice injected with losartan subcutaneously without pump (Group 3, both day 14 and day 19).

Also as shown in Table 3, the plasma and tumor levels of losartan carboxylic acid were generally higher in the mice administered with losartan via subcutaneous pump (Group 2, 6-hour time point), as compared to the mice injected with losartan subcutaneously without pump (Group 3, 6-hour time point), on day 19. Table 3 also indicates that the plasma and tumor levels of losartan carboxylic acid increased or became steady from the 2-hour time point to the 6-hour time point in the mice administered with losartan via subcutaneous pump (Group 2, both day 14 and day 19), whereas the plasma and tumor levels of losartan carboxylic acid decreased from the 2-hour time point to the 6-hour time point in the mice injected with losartan subcutaneously without pump (Group 3, both day 14 and day 19).

These data indicate that continuous administration of losartan (e.g., subcutaneous pump administration) resulted in generally higher plasma and tumor levels of losartan and losartan carboxylic acid, as compared to pulsatile administration (e.g., subcutaneous injections without pump).

TABLE 3
Measurement of losartan and losartan carboxylic acid levels in plasma and
tumor samples from mice implanted with AK4.4 pancreatic tumors

| | | | | |
|---|---|---|---|---|
| | Plasma | Tumor | Plasma | Tumor |
| Group | 2 hr | 2 hr | 6 hr | 6 hr |

Day 14 Losartan (ng/mL)

| | | | | |
|---|---|---|---|---|
| 2 | 227 | 50 | 304 | 57 |
| 3 | 423 | 163 | 11 | 21 |

Day 19 Losartan (ng/mL)

| | | | | |
|---|---|---|---|---|
| 2 | 370 | 56.1 | 376 | 66 |
| 3 | 152 | 41 | 28 | 54 |

Day 14 Losartan Carboxylic
Acid (ng/mL)

| | | | | |
|---|---|---|---|---|
| 2 | 35 | 14 | 90 | 12 |
| 3 | 1156 | 293 | 81 | 22 |

Day 19 Losartan Carboxylic
Acid (ng/mL)

| | | | | |
|---|---|---|---|---|
| 2 | 159 | 28.7 | 158 | 23 |
| 3 | 700 | 152 | 74 | 11 |

### Tumor Volume Measurement

The volumes of the tumors collected from the mice in Group 1 (subcutaneous injections of PBS as a control), Group 2 (subcutaneous pump administration of losartan at 40 mg/kg), and Group 3 (subcutaneous injections of losartan without pump at 40 mg/kg) were measured on day 19. The baseline tumor volumes were measured on day 7.

The changes in the average tumor volume in the mouse AK4.4 pancreatic tumor model are shown in Table 4. As shown in Table 4, the average tumor volume in Group 2 is about 27% smaller than that of Group 1 and Group 3 on day 19. The average tumor volume increased by 21-fold from day 7 to day 19 in Group 2 mice, whereas the average tumor volume increased more than 30-fold in Group 1 and Group 3 mice during the same time period (Table 4).

These data indicate that continuous administration of losartan (e.g., subcutaneous pump administration) was more effective in reducing tumor volume than pulsatile administration (e.g., subcutaneous injections without pump).

TABLE 4
Tumor volume measurement in mice treated with losartan or vichile alone

| Treatment | Average volume (Day 7) | Average volume (Day 19) |
|---|---|---|
| Group | mm³ | mm³ |
| 1 | 10.63 | 321.84 |
| 2 | 11.17 | 235.98 |
| 3 | 10.33 | 323.13 |

### Example 12

### Collagen I Immunofluorescence Quantitative Image Analysis

This example presents a study that compared the pump administration and pulsatile injections of losartan based on the results of immunofluorescence quantitative image analysis for collagen I in AK4.4 pancreatic tumors.

### Experimental Design

Mice implanted with AK4.4 pancreatic tumors were randomized into three groups. In Group 1, the mice were subcutaneously injected with saline as a control. In Group 2, losartan was administered to the mice via subcutaneous pump. In Group 3, losartan was administered to the mice via subcutaneous injections in the absence of pump.

Immunohistochemistry for collagen I was performed as described in the experimental protocols for Examples 11.

### Image Analysis Approach

To quantify the positively stained area for collagen I and to identify the number of collagen I positive fibers, each collagen fiber was outlined and each collagen area was calculated from the collagen I positive area/total area. The average collagen I area and number of collagen I positive fibers were calculated by averaging the total ten fields from each tumor sample.

### Results

Representative images of collagen I staining are shown in FIG. 37A-37C. As shown in FIG. 37A, the percentage of collagen I positive area in a tumor sample from Group 2 is 4.453%, as compared to 22.356% (FIG. 37A) in a sample from Group 2 and 11.34% (FIG. 37C) in a sample from Group 3.

The average percentages of collagen I positive areas in tumor samples from Groups 1-3 mice are summarized in FIG. 38A. As shown in FIG. 38A, the tumor samples from the mice administered with losartan via subcutaneous pump (Group 2) showed an average of 3.24% positive staining for collagen I, lower than 16.26% observed in the samples from the control mice subcutaneously injected with PBS (Group 1) and 8.71% observed in the samples from the mice subcutaneously injected with losartan in the absence of pump (Group 3).

The average numbers of collagen I positive fibers in tumor samples from Groups 1-3 mice are summarized in FIG. 38B. As shown in FIG. 38B, the tumor samples from the mice administered with losartan via subcutaneous pump (Group 2) showed an average of 10.01 collagen I fibers per image, lower than 28.04 observed in the samples from the control mice subcutaneously injected with PBS (Group 1) and 17.93 observed in the samples from the mice subcutaneously injected with losartan in the absence of pump (Group 3).

The statistical analysis of these results is shown in Table 5.

TABLE 5
Statistical analysis of the results for the percentages of
collagen I positive areas and the number of collagen I fibers

| % Area of | | % Area | |
|---|---|---|---|
| Collagen I | T-Test | of Collagen I | T-Test |
| Group 1_PBS | 1.18E−05 | Group 1_PBS | 0.00067837 |
| Group 2_Losartan | | Group 3_Losartan | |
| Pump | | | |
| Number of | | Number of | |
| Collagen I | | collagen I | |
| Fibers | T-Test | fibers | T-Test |
| Group 1_PBS | 2.20287E−06 | Group 1_PBS | 0.0001135 |
| Group 2_Losartan | | Group 3_Losartan | |
| Pump | | | |

These data indicate that administration of losartan to mice implanted with pancreatic tumors reduced collagen production in tumors, as evidenced by the percentage of collagen I positive area and the number of collagen I fibers. Further, continuous administration of losartan (e.g., subcutaneous pump administration) showed a more significant effect on reducing collagen production, as compared to pulsatile administration (e.g., subcutaneous injections without pump). This study suggests that AHCM, such as losartan, can be used to improve the delivery or efficacy of cancer therapy.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of improving the delivery or efficacy of a therapy, in a subject, comprising:
optionally, identifying the subject as being in need of receiving an anti-hypertensive and/or a collagen modifying agent (“AHCM”) on the basis of the need for improved delivery or efficacy of the therapy; and
any of (a), (b), (c), or all:
(a) administering the AHCM to the subject;
(b) administering the therapy; or
(c) administering a microenvironment modulator,
thereby improving the delivery or efficacy of the therapy, in the subject.

2. A method of treating or preventing a cancer, in a subject, comprising:
identifying the subject as being in need of receiving an anti-hypertensive and/or a collagen modifying agent (“AHCM”) on the basis of the need for improved delivery or efficacy of a cancer therapy; and any of (a), (b), (c), or all:
(a) administering the AHCM to the subject;
(b) administering the cancer therapy; or
(c) administering a microenvironment modulator;
wherein the AHCM and/or microenvironment modulator is administered in a dosage sufficient to treat or prevent the cancer.

3. The method of claim 1 or 2, wherein the method results in, or comprises, an improvement of a disorder- or cancer-related parameter in said subject, as compared to a subject treated with said therapy but without administration of the AHCM and/or microenvironment modulator.

4. The method of claim 3, wherein said parameter is chosen from one or more of:
a) objective response rate (ORR);
b) progression free survival (PFS);
c) overall survival (OS);
d) reduction in toxicity;
e) drug concentration at a disorder or disease site;
f) tumor response;
g) blood perfusion at a disorder or disease site;
h) oxygenation at a disorder or disease site;
i) interstitial fluid pressure at a disorder or disease site; or
j) the level of extracellular matrix content or composition.

5. (canceled)

6. The method of claim 1 or 2, which comprises one or more of the following:
a) administering the AHCM, the therapy or the cancer therapy, or both, as an entity having a hydrodynamic diameter of greater than 1, 5, 10, 15, 20, 25, 30, 35, 45, 50, 75, 100, 150, 200 nm, but less than 300 nm;
b) the subject has not been administered a dose of the AHCM within 5, 10, 30, 60 or 100 days of the diagnosis of the cancer or the initiation of the AHCM dosing;
c) the subject is not hypertensive, or has been hypertensive, prior to administration of the AHCM;
d) the AHCM and/or microenvironment modulator is administered at least one, two, three, or five days; or one, two, three, four, five or more weeks, prior to the therapy or the cancer therapy;
e) the AHCM and/or microenvironment modulator is administered at least one, two, three, or five days; or one, two, three, four, five or more weeks, prior to the therapy or the cancer therapy, e.g., the cancer therapy, and concurrently with the therapy or the cancer therapy,
f) the AHCM and/or microenvironment modulator is administered continuously over a period of at least 1, 5, 10, or 24 hours; at least 2, 5, 10, or 14 days; at least 2, 3, 4, 5 or 6 weeks; at least 2, 3, 4, 5 or 6 months; or at least 1, 2, 3, 4 or 5 years, or
g) the AHCM and/or microenvironment modulator is administered after cessation of the therapy or the cancer therapy;
h) at least days, weeks, months or years after cessation of the therapy or the cancer therapy.

7. The method of claim 1 or 2, wherein the AHCM is chosen from one or more of:
(i) an angiotensin II receptor blocker (AT₁ blocker),
(ii) an antagonist of renin angiotensin aldosterone system (“RAAS antagonist”),
(iii) an angiotensin converting enzyme (ACE) inhibitor,
(iv) a thrombospondin 1 (TSP-1) inhibitor,
(v) a transforming growth factor beta 1 (TGF-β1) inhibitor,
(vi) a stromal cell-derived growth factor 1 alpha (SDF-1a) inhibitor or
(vii) a connective tissue growth factor (CTGF) inhibitor.

8. The method of claim 1 or 2, wherein the AHCM is an AT₁ inhibitor chosen from one or more of: losartan, candesartan, eprosartan mesylate, EXP 3174, irbesartan, L158,809, olmesartan, saralasin, telmisartin, valsartan, or a derivative thereof.

9. The method of claim 1 or 2, wherein the AHCM is losartan.

10. The method of claim 1 or 2, wherein the AHCM is a RAAS antagonist chosen from one or more of: aliskiren (TEKTURNA®, RASILEZ®), remikiren (Ro 42-5892), enalkiren (A-64662), SPP635, or a derivative thereof.

11. The method of claim 1 or 2, wherein the AHCM is an ACE inhibitor chosen from one or more of: benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), trandolapril (MAVIK®), or a derivative thereof.

12. The method of claim 1 or 2, wherein the AHCM is a TSP-1 inhibitor chosen from one or more of: ABT-510, CVX-045, LSKL, or a derivative thereof.

13. The method of claim 7, wherein the TGF-β1 inhibitor is chosen from one or more of: an anti-TGF-β1 antibody, or a TGF-β1 peptide inhibitor.

14. The method of claim 7, wherein the CTGF inhibitor is chosen from one or more of: DN-9693, FG-3019, or a derivative thereof.

15. The method of claim 1 or 2, wherein the microenvironment modulator is chosen from one or more of an anti-angiogenic therapy; an inhibitor of vascular endothelial growth factor (VEGF) pathway; an agent that decreases the level or production of hyaluronic acid; an inhibitor of the hedgehog pathway; a disulfide-based cyclic RGD peptide peptide (iRGD) or an analogue thereof; a taxane therapy; an agent that decreases the level or production of collagen or procollagen; an anti-fibrotic agent; or a profibrotic pathway inhibitor.

16. The method of claim 1 or 2, wherein the AHCM and/or microenvironment modulator is administered in an amount sufficient to enhance the distribution or efficacy of the therapy or the cancer therapy.

17. The method of claim 1 or 2, wherein the AHCM and/or microenvironment modulator is administered at a dose that causes one or more of: decreases the level or production of collagen, decreases tumor fibrosis, reduces interstitial fluid pressure, increases interstitial tumor transport, improves tumor perfusion, increases tumor oxygenation; decreases tumor hypoxia; decreases tumor acidosis; enables immune cell infiltration; decreases immunosuppression; increases antitumor immunity; decreases cancer stem cells (also referred to herein as tumor-initiating-cells); or enhances penetration or diffusion, of the cancer therapy in a tumor or tumor vasculature, in the subject.

18. The method of claim 1 or 2, wherein the AHCM is losartan, and is administered at 25-100 mg day.

19.-20. (canceled)

21. The method of claim 1 or 2, wherein the AHCM is losartan, and is administered at a dose that is greater than 1.1, 1.5, 1.7, 2, 3, 4, 5, 10-fold or higher, that of the standard of care dose for anti-hypertensive or anti-heart failure use.

22. (canceled)

23. The method of claim 1 or 2, wherein the AHCM is administered as an entity having a hydrodynamic diameter of greater than 1, 5, 10, 15, 20, 25, 30, 35, 45, 50, 75, 100, 150, 200 nm, but less than 300 nm.

24. The method of claim 23, wherein the AHCM is administered as a polymeric nanoparticle or a lipid nanoparticle.

25. The method of claim 1 or 2, wherein the therapy or the cancer therapy is a therapeutic or a cancer therapeutic that is administered as an entity having a hydrodynamic diameter of greater than 1, 5, 10, 15, 20, 25, 30, 35, 45, 50, 75, 100, 150, 200 nm, but less than 300 nm.

26. The method of claim 25, wherein the therapeutic or the cancer therapeutic is administered as a polymeric nanoparticle or a lipid nanoparticle.

27. The method of claim 1 or 2, wherein the AHCM, the microenvironment modulator, or a therapeutic or a cancer therapeutic, each independently, is provided as an entity having the following size ranges (in nm): a hydrodynamic diameter of less than or equal to 1, or between 0.1 and 1.0 nm; a hydrodynamic diameter of between 5 and 20, or 5 and 15 nm; or a hydrodynamic diameter of 1, 5, 10, 15, 20, 25, 30, 35, 45, 50, 75, 100, 150, 200 nm, but less than 300 nm.

28.-31. (canceled)

32. The method of claim 1, wherein the subject is in need of, or is being considered for, cancer therapy.

33. The method of claim 1 or 2, which comprises the step of determining if the subject has a cancer or has a tumor expressing an angiotensin receptor, and, responsive to said determination, administering the AHCM and/or microenvironment modulator, and the cancer therapy.

34. The method of claim 1 or 2, wherein the subject has a pre-neoplastic condition or a pre-disposition to cancer.

35. The method of claim 1 or 2, wherein the subject is at risk of having, or has a solid, fibrotic tumor.

36. The method of claim 1 or 2, wherein the subject has a tumor containing an extracellular matrix component chosen from collagen, procollagen and/or hyaluronan (HA).

37. The method of claim 2, wherein the cancer is chosen from one or more of pancreatic, breast, colorectal, colon, lung, skin, ovarian, prostate, cervix, gastric, gastrointestinal, stomach, head and neck, kidney, liver cancer, brain, or a metastatic lesion thereof

38. The method of claim 1 or 2, wherein the AHCM and/or the microenvironment modulator;
(i) is administered prior to the therapy or the cancer therapy;
(ii) is administered at least one, two, three, or five days; or one, two, three, four, five or more weeks, prior to the therapy or the cancer therapy;
(iii) is maintained for a preselected portion of the time the subject receives the therapy or the cancer therapy;
(iv) is maintained for the entire period in which the therapy or the cancer therapy is administered; or
(v) is administered after cessation of the therapy or the cancer therapy.

39.-42. (canceled)

43. The method of claim 1 or 2, wherein the AHCM and/or the microenvironment modulator is administered continuously over a period of at least 1, 5, 10, or 24 hours; at least 2, 5, 10, or 14 days; at least 2, 3, 4, 5 or 6 weeks; at least 2, 3, 4, 5 or 6 months; or at least 1, 2, 3, 4 or 5 years.

44. (canceled)

45. The method of claim 1 or 2, wherein the AHCM is formulated for oral, subcutaneous, intravenous continuous delivery; or is administered as a sustained release formulation.

46. The method of claim 1 or 2, wherein the AHCM is administered via a subcutaneous pump, an implant or a depot.

47. The method of claim 2, wherein the cancer therapy is chosen from one or more of:
(i) a cytotoxic or a cytostatic agent;
(ii) a cancer therapeutic chosen from a viral cancer therapeutic agent, a lipid nanoparticle of an anti-cancer therapeutic agent, a polymeric nanoparticle of an anti-cancer therapeutic agent, an antibody against a cancer target, a dsRNA agent, an antisense RNA agent, or a chemotherapeutic agent;
(iii) an immunotherapy, an immune-cell therapy, or adoptive immunotherapy;
(iv) radiation,
(v) surgery,
(vi) a photodynamic therapy; or
(viii) any combination of (i)-(vi).

48. The method of claim 47, wherein:
(i) the lipid nanoparticle is chosen from pegylated liposomal doxorubicin or liposomal paclitaxel;
(ii) the antibody against the cancer target is chosen from an antibody against HER-2/neu, HER3, VEGF, or EGFR;
(iii) the chemotherapeutic agent is chosen from an antimicrotubule agent, a topoisomerase inhibitor, a taxane, an antimetabolite, a mitotic inhibitor, an alkylating agent, an intercalating agent, an anti-angiogenic agent, a vascular targeting agent or a vascular disrupting agent; or
(iv) the cancer therapy is a tyrosine kinase inhibitor chosen from sunitinib, erlotinib, gefitinib, sorafenib, icotinib, lapatinib, neratinib, vandetanib, BIBW 2992 or XL-647, or an anti-EGFR antibody chosen from cetuximab, panitumumab, zalutumumab, nimotuzumab necitumumab or matuzumab.

49. The method of claim 47, wherein the chemotherapeutic agent is chosen from gemcitabine, cisplatin, epirubicin, 5-fluorouracil, paclitaxel, oxaliplatin, or leucovorin.

50.-54. (canceled)

55. The method of claim 1 or 2, wherein the AHCM, the microenvironment modulator, or the therapy or the cancer therapy is administered to the subject by a systemic administration chosen from oral, parenteral, subcutaneous, intravenous, rectal, intramuscular, intraperitoneal, intranasal, transdermal, or by inhalation or intracavitary installation.

56. The method of claim 1 or 2, further comprising evaluating or monitoring the subject, for one or more of:
tumor size;
the level or signaling of one or more of transforming growth factor beta 1 (TGFb1), connective tissue growth factor (CTGF), or thrombospondin-1 (TSP-1);
the level or expression of an angiotensin receptor;
tumor collagen I levels;
fibrotic content,
interstitial pressure;
a biomarker chosen from collagen I, collagen III, collagen IV, TGFb1, CTGF, or TSP-1;
levels of one or more cancer markers;
the rate of appearance of new lesions, metabolism, hypoxia evolution;
the appearance of new disease-related symptoms;
the size of tissue mass;
amount of disease associated pain;
histological analysis, lobular pattern, and/or the presence or absence of mitotic cells; or
tumor aggressivity, vascularization of primary tumor, or metastatic spread.

57. A pharmaceutical composition comprising a nanoparticle comprising an AHCM, wherein the AHCM is chosen from one or more of:
(i) an angiotensin II receptor blocker (AT₁ blocker),
(ii) an antagonist of renin angiotensin aldosterone system (RAAS antagonist),
(iii) an angiotensin converting enzyme (ACE) inhibitor,
(iv) a thrombospondin 1 (TSP-1) inhibitor,
(v) a transforming growth factor beta 1 (TGF-β1) inhibitor,
(vi) a stromal cell-derived growth factor 1 alpha (SDF-1a) inhibitor or
(vii) a connective tissue growth factor (CTGF) inhibitor, and
wherein the nanoparticle has a hydrodynamic diameter of greater than 1, 5, 10, 15, 20, 25, 30, 35, 45, 50, 75, 100, 150, 200 nm, but less than 300 nm.

58. The pharmaceutical composition of claim 57, further comprising a microenvironment modulator, and/or a therapeutic agent or a cancer therapeutic agent.

59. The pharmaceutical composition of claim 58, wherein the cancer therapeutic agent is chosen from a viral cancer therapeutic agent, a lipid nanoparticle of an anti-cancer agent, a polymeric nanoparticle of an anti-cancer agent, an antibody against a cancer target, a dsRNA agent, an antisense RNA agent, or a chemotherapeutic agent.

60. The pharmaceutical composition of claim 57, wherein the nanoparticle is a polymeric nanoparticle or a lipid nanoparticle.

61.-62. (canceled)

63. The pharmaceutical composition of claim 57, wherein the AHCM is formulated in a dosage form that is greater than 1.1, 1.5, 1.7, 2, 3, 4, 5, 10-fold or higher, that of the standard of care dosage form for anti-hypertensive or anti-heart failure use of the AHCM.

64. (canceled)

65. A dosage form of an AHCM, wherein the AHCM is formulated in a dosage form that is greater than 1.1, 1.5, 1.7, 2, 3, 4, 5, 10-fold or higher, that of the standard of care dosage form for anti-hypertensive or anti-heart failure use of the AHCM.

66. A method optimizing access to a cancer, or optimizing delivery to a cancer of an agent, e.g., a diagnostic or imaging agent, comprising:
administering an anti-hypertensive and/or a collagen modifying agent (“AHCM”) to the subject; and
optionally, administering the agent to said subject, wherein the method comprises one or more of the following:
a) the diagnostic or imaging agent has a hydrodynamic diameter of greater than 1, 5, or 20-150 nm;
b) the agent is a radiologic agent, an NMR agent, a contrast agent; or
c) the subject is treated with a dosing of AHCM administration, which is initiated prior to administration of the agent for at least two, three, or five days, or one, two, three, four, five or more weeks prior to administration of the agent.

67. A method, or assay for, identifying an anti-hypertensive and/or a collagen modifying (AHCM), comprising:
contacting a cancer or cancer-associated cell with a candidate agent;
detecting a change in the cancer cell in the presence, or absence, of the candidate agent, wherein the detected change includes one or more of: an increase or decrease of activated TGF beta, TGF beta 1 level, connective tissue growth factor (CTGF) level, or collagen level, wherein the candidate agent is chosen from one or more of: an antagonist of renin angiotensin aldosterone system (RAAS antagonist), an angiotensin converting enzyme (ACE) inhibitor, an angiotensin II receptor blocker (AT₁ blocker), a thrombospondin 1 (TSP-1) inhibitor, a transforming growth factor beta 1 (TGF-(1) inhibitor, or a connective tissue growth factor (CTGF) inhibitor.

68.-71. (canceled)

72. The method, or assay, of claim 67, comprising evaluating the candidate agent in vitro by adding the candidate agent to the culture medium; and the condition medium is analyzed for an increase or decrease of: activated TGF beta, TGFb1 level, connective tissue growth factor (CTGF) level, or collagen or hyaluronan level.

73. The method, or assay, of claim 67, comprising administering the candidate agent to an animal tumor model; and analyzing the subject for an increase or decrease of: activated TGF beta, TGFb1 level, connective tissue growth factor (CTGF) level, or collagen level.

74. (canceled)

75. A therapeutic kit comprising an anti-hypertensive and/or a collagen modifying (AHCM), alone or in combination with a microenvironment modulator, and/or a cancer therapy, and instructions for use for the treatment of cancer.

76. A diagnostic kit comprising an anti-hypertensive and/or a collagen modifying (AHCM), alone or in combination with an imaging agent, and instructions for use for the diagnosis of cancer.

77. A method of selecting a subject for receiving an anti-hypertensive and/or a collagen modifying agent (“AHCM”), comprising:
selecting the subject as being in need of receiving the AHCM on the basis of the need for improved delivery or efficacy of the cancer therapy; and either (a), (b), or both:
(a) administering the AHCM to the subject; or
(b) administering the cancer therapy,
wherein the AHCM is administered in a dosage sufficient to improve the delivery or efficacy of the cancer therapy.
